SEQUENCE LISTING (65 sequences)

SEQ ID NO: 1 - DNA, 212 bp, Artificial sequence
  Synthetic construct
gggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 2 - DNA, 584 bp, Artificial sequence
  Synthetic construct
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 3 - DNA, 1211 bp, Artificial sequence
  Synthetic construct
ggcctccgcgccgggttttggcgcctcccgcgggcgcccccctcctcacggcgagcgctgccacgtcagacgaagggcgcaggagcgttcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagttgcgggctgctgggctggccggggctttcgtggccgccgggccgctcggtgggacggaagcgtgtggagagaccgccaagggctgtagtctgggtccgcgagcaaggttgccctgaactgggggttggggggagcgcacaaaatggcggctgttcccgagtcttgaatggaagacgcttgtaaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcgggtttgtcgtctggttgcgggggcggcagttatgcggtgccgttgggcagtgcacccgtacctttgggagcgcgcgcctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagac

SEQ ID NO: 4 - DNA, 511 bp, Artificial sequence
  Synthetic construct
ggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccag

SEQ ID NO: 5 - DNA, 624 bp, Artificial sequence
  Synthetic construct
gaattctgtcattttactagggtgatgaaattcccaagcaacaccatccttttcagataagggcactgaggctgagagaggagctgaaacctacccggggtcaccacacacaggtggcaaggctgggaccagaaaccaggactgttgactgcagcccggtattcattctttccatagcccacagggctgtcaaagaccccagggcctagtcagaggctcctccttcctggagagttcctggcacagaagttgaagctcagcacagccccctaacccccaactctctctgcaaggcctcaggggtcagaacactggtggagcagatcctttagcctctggattttagggccatggtagagggggtgttgccctaaattccagccctggtctcagcccaacaccctccaagaagaaattagaggggccatggccaggctgtgctagccgttgcttctgagcagattacaagaagggactaagacaaggactcctttgtggaggtcctggcttagggagtcaagtgacggcggctcagcactcacgtgggcagtgccagcctctaagagtgggcaggggcactggccacagagtcccagggagtcccaccagcctagtcgccagacc

SEQ ID NO: 6 - DNA, 1678 bp, Artificial sequence
  Synthetic construct
cacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 7 - DNA, 315 bp, Artificial sequence
  Synthetic construct
gagtttactccctatcagtgatagagaacgtatgtcgagtttactccctatcagtgatagagaacgatgtcgagtttactccctatcagtgatagagaacgtatgtcgagtttactccctatcagtgatagagaacgtatgtcgagtttactccctatcagtgatagagaacgtatgtcgagtttatccctatcagtgatagagaacgtatgtcgagtttactccctatcagtgatagagaacgtatgtcgaggtaggcgtgtacggtgggaggcctatataagcagagctcgtttagtgaaccgtcagatcgcc

SEQ ID NO: 8 - DNA, 250 bp, Artificial sequence
  Synthetic construct
cgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttatcgtaacttgaaagtatttcgatttcttggctttatatatcttgtggaaaggacgaaacacc

SEQ ID NO: 9 - protein, 718 aa, Artificial sequence
  Synthetic construct
LPTCSCLDRVIQKDKGPYYTHLGAGPSVAAVREIMENRYGQKGNAIRIEIVVYTGKEGKSSHGCPIAKWVLRRSSDEEKVLCLVRQRTGHHCPTAVMVVLIMVWDGIPLPMADRLYTELTENLKSYNGHPTDRRCTLNENRTCTCQGIDPETCGASFSFGCSWSMYFNGCKFGRSPSPRRFRIDPSSPLHEKNLEDNLQSLATRLAPIYKQYAPVAYQNQVEYENVARECRLGSKEGRPFSGVTACLDFCAHPHRDIHNMNNGSTVVCTLTREDNRSLGVIPQDEQLHVLPLYKLSDTDEFGSKEGMEAKIKSGAIEVLAPRRKKRTCFTQPVPRSGKKRAAMMTEVLAHKIRAVEKKPIPRIKRKNNSTTTNNSKPSSLPTLGSNTETVQPEVKSETEPHFILKSSDNTKTYSLMPSAPHPVKEASPGFSWSPKTASATPAPLKNDATASCGFSERSSTPHCTMPSGRLSGANAAAADGPGISQLGEVAPLPTLSAPVMEPLINSEPSTGVTEPLTPHQPNHQPSFLTSPQDLASSPMEEDEQHSEADEPPSDEPLSDDPLSPAEEKLPHIDEYWSDSEHIFLDANIGGVAIAPAHGSVLIECARRELHATTPVEHPNRNHPTRLSLVFYQHKNLNKPQHGFELNKIKFEAKEAKNKKMKASEQKDQAANEGPEQSSEVNELNQIPSHKALTLTHDNVVTVSPYALTHVAGPYNHWV

SEQ ID NO: 10 - protein, 617 aa, Artificial sequence
  Synthetic construct
IFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQD

SEQ ID NO: 11 - protein, 523 aa, Artificial sequence
  Synthetic construct
DALDDFDLDMLGSDALDDFDLDMLGSDALDDFDLDMLGSDALDDFDLDMLINSRSSGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 12 - protein, 248 aa, Artificial sequence
  Synthetic construct
MSRLDKSKVINGALELLNGVGIEGLTTRKLAQKLGVEQPTLYWHVKNKRALLDALPIEMLDRHHTHFCPLEGESWQDFLRNNAKSYRCALLSHRDGAKVHLGTRPTEKQYETLENQLAFLCQQGFSLENALYALSAVGHFTLGCVLEEQEHQVAKEERETPTTDSMPPLLRQAIELFDRQGAEPAFLFGLELIICGLEKQLKCESGGPTDALDDFDLDMLPADALDDFDLDMLPADALDDFDLDMLPG

SEQ ID NO: 13 - protein, 1367 aa, Artificial sequence
  Synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 14 - protein, 1053 aa, Artificial sequence
  Synthetic construct
MKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 15 - protein, 1228 aa, Artificial sequence
  Synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 16 - DNA, 83 bp, Artificial sequence
  Synthetic construct
gttttagagctagaaatagcaagttaaaataaggctagtccgttatcaacttgaaaaagtggcaccgagtcggtgcttttttt

SEQ ID NO: 17 - DNA, 76 bp, Artificial sequence
  Synthetic construct
gttttagtactctggaaacagaatctactaaaacaaggcaaaatgccgtgtttatctcgtcaacttgttggcgaga

SEQ ID NO: 18 - DNA, 24 bp, Artificial sequence
  Synthetic construct
aaacaccgaatttctactaagtgt

SEQ ID NO: 19 - protein, 18 aa, Artificial sequence
  Synthetic construct
EGRGSLLTCGDVEENPGP

SEQ ID NO: 20 - protein, 19 aa, Artificial sequence
  Synthetic construct
ATNFSLLKQAGDVEENPGP

SEQ ID NO: 21 - protein, 20 aa, Artificial sequence
  Synthetic construct
QCTNYALLKLAGDVESNPGP

SEQ ID NO: 22 - protein, 22 aa, Artificial sequence
  Synthetic construct
VKQTLNFDLLKLAGDVESNPGP

SEQ ID NO: 23 - DNA, 34 bp, Artificial sequence
  Synthetic construct
ataacttcgtatagcatacattatacgaagttat

SEQ ID NO: 24 - protein, 25 aa, Artificial sequence
  Synthetic construct
MGVLLTQRTLLSLVLALLFPSMASM

SEQ ID NO: 25 - protein, 16 aa, Artificial sequence
  Synthetic construct
MKCLLYLAFLFIGVNC

SEQ ID NO: 26 - protein, 21 aa, Artificial sequence
  Synthetic construct
METDTLLLWVLLLWVPGSTGD

SEQ ID NO: 27 - protein, 19 aa, Artificial sequence
  Synthetic construct
MGWSCIILFLVATATGVHS

SEQ ID NO: 28 - protein, 17 aa, Artificial sequence
  Synthetic construct
MRAWIFFLLCLAGRALA

SEQ ID NO: 29 - protein, 21 aa, Artificial sequence
  Synthetic construct
MWWRLWWLLLLLLLLWPMVWA

SEQ ID NO: 30 - protein, 22 aa, Artificial sequence
  Synthetic construct
MDMRVPAQLLGLLLLWLRGARC

SEQ ID NO: 31 - protein, 16 aa, Artificial sequence
  Synthetic construct
MPLLLLLPLLWAGALA

SEQ ID NO: 32 - protein, 23 aa, Artificial sequence
  Synthetic construct
MDAMKRGLCCVLLLCGAVFVSPS

SEQ ID NO: 33 - protein, 18 aa, Artificial sequence
  Synthetic construct
MAFLWLLSCWALLGTTFG

SEQ ID NO: 34 - protein, 15 aa, Artificial sequence
  Synthetic construct
MNLLLILTFVAAAVA

SEQ ID NO: 35 - protein, 20 aa, Artificial sequence
  Synthetic construct
MYRMQLLSCIALSLALVTNS

SEQ ID NO: 36 - protein, 17 aa, Artificial sequence
  Synthetic construct
MGVKVLFALICIAVAEA

SEQ ID NO: 37 - protein, 16 aa, Artificial sequence
  Synthetic construct
MKWVTFISLLFSSAYS

SEQ ID NO: 38 - protein, 16 aa, Artificial sequence
  Synthetic construct
MKTIIALSYIFCLVLG

SEQ ID NO: 39 - protein, 24 aa, Artificial sequence
  Synthetic construct
MALWMRLLPLLALLALWGPDPAAA

SEQ ID NO: 40 - protein, 16 aa, Artificial sequence
  Synthetic construct
MKPIFLVLLVVTSAYA

SEQ ID NO: 41 - protein, 199 aa, Artificial sequence
  Synthetic construct
MTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 42 - protein, 264 aa, Artificial sequence
  Synthetic construct
MIEQDGLHAGSPAAWVERLFGYDWAQQTIGCSDAAVFRLSAQGRPVLFVKTDLSGALNELQDEAARLSWLATTGVPCAAVLDVVTEAGRDWLLLGEVPGQDLLSSHLAPAEKVSIMADAMRRLHTLDPATCPFDHQAKHRIERARTRMEAGLVDQDDLDEEHQGLAPAELFARLKASMPDGEDLVVTHGDACLPNIMVENGRFSGFIDCGRLGVADRYQDIALATRDIAEELGGEWADRFLVLYGIAAPDSQRIAFYRLLDEFF

SEQ ID NO: 43 - protein, 132 aa, Artificial sequence
  Synthetic construct
MAKPLSQEESTLIERATATINSIPISEDYSVASAALSSDGRIFTGVNVYHFTGGPCAELVVLGTAAAAAAGNLTCIVAIGNENRGILSPCGRCRQVLLDLHPGIKAIVKDSDGQPTAVGIRELLPSGYVWEG

SEQ ID NO: 44 - DNA, 1182 bp, Artificial sequence
  Synthetic construct
atgacggacagacagacagacaccgcccccagccccagcgcccacctcctcgccggcgggcagccgacggtggacgcggcggcgagccgcgagcaggagccgaagcccgcgcccggaggcggggtggagggggtcggggctcgcgggattgcacggaaacttttcgtccaacttctgggctcttctctctccggagtagccgtggtctgcgccgcaggaggcaaaccgatcggagctgggagaagtgctagctcgggcctggagaagccggggcccgagaagagaggggagaaagagaaggaagaggagagggggccgcagtgggcgctcggctctcgggagccgggctcatggacgggtgaggcggcggtgtgcgcagacagtgctccagccgcgcgcgcgccccaggccccggcccgggcctcggttccagaagggagaggagcccgccaaggcgcgcaagagagcgggctgcctcgcagtccgagccggagagggagcgcgagccgcgccggccccggacgggcctctgaaaccatgaactttctgctctcttgggtgcactggaccctggctttactgctgtacctccaccatgccaagtggtcccaggctgcacccacgacagaaggggagcagaaagcccatgaagtggtgaagttcatggacgtctaccagcgcagctattgccgtccaattgagaccctggtggacatcttccaggagtaccccgatgagatagagtatatcttcaagccgtcctgtgtgcccctaatgcggtgtgcgggctgctgcaatgatgaagccctggagtgcgtgcccacgtcggagagcaacgtcactatgcagatcatgcggatcaaacctcaccaaagccagcacataggagagatgagcttcctgcagcatagcagatgtgaatgcagaccaaagaaagatagaacaaagccagaaaaaaaatcagttcgaggaaagggaaagggtcaaaaacgaaagcgcaagaaatcccggtttaaatcctggagcgttcactgtgagccttgttcagagcggagaaagcatttgtttgtccaagatccgcagacgtgtaaatgttcctgcaaaaacacagactcgcgttgcaaggcgaggcagcttgagttaaacgaacgtacttgcagatgtgacaagccaaggcggtga

SEQ ID NO: 45 - DNA, 1173 bp, Artificial sequence
  Synthetic construct
atgccgccctcggggctgcggctactgccgcttctgctcccactcccgtggcttctagtgctgacgcccgggaggccagccgcgggactctccacctgcaagaccatcgacatggagctggtgaaacggaagcgcatcgaagccatccgtggccagatcctgtccaaactaaggctcgccagtcccccaagccagggggaggtaccgcccggcccgctgcccgaggcggtgctcgctttgtacaacagcacccgcgaccgggtggcaggcgagagcgccgacccagagccggagcccgaagcggactactatgctaaagaggtcacccgcgtgctaatggtggaccgcaacaacgccatctatgagaaaaccaaagacatctcacacagtatatatatgttcttcaatacgtcagacattcgggaagcagtgcccgaacccccattgctgtcccgtgcagagctgcgcttgcagagattaaaatcaagtgtggagcaacatgtggaactctaccagaaatatagcaacaattcctggcgttaccttggtaaccggctgctgacccccactgatacgcctgagtggctgtcttttgacgtcactggagttgtacggcagtggctgaaccaaggagacggaatacagggctttcgattcagcgctcactgctcttgtgacagcaaagataacaaactccacgtggaaatcaacgggatcagccccaaacgtcggggcgacctgggcaccatccatgacatgaaccggcccttcctgctcctcatggccacccccctggaaagggcccagcacctgcacagctcacggcaccggagagccctggataccaactattgcttcagctccacagagaagaactgctgtgtgcggcagctgtacattgactttaggaaggacctgggttggaagtggatccacgagcccaagggctaccatgccaacttctgtctgggaccctgcccctatatttggagcctggacacacagtacagcaaggtccttgccctctacaaccaacacaacccgggcgcttcggcgtcaccgtgctgcgtgccgcaggctttggagccactgcccatcgtctactacgtgggtcgcaagcccaaggtggagcagttgtccaacatgattgtgcgctcctgcaagtgcagctga

SEQ ID NO: 46 - DNA, 462 bp, Artificial sequence
  Synthetic construct
atgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttga

SEQ ID NO: 47 - DNA, 462 bp, Artificial sequence
  Synthetic construct
atgggtctcacctcccaactgcttccccctctgttcttcctgctagcatgtgccggcaactttgtccacggacacaagtgcgatatcaccttacaggagatcatcaaaactttgaacagcctcacagagcagaagactctgtgcaccgagttgaccgtaacagacatctttgctgcctccaagaacacaactgagaaggaaaccttctgcagggctgcgactgtgctccggcagttctacagccaccatgagaaggacactcgctgcctgggtgcgactgcacagcagttccacaggcacaagcagctgatccgattcctgaaacggctcgacaggaacctctggggcctggcgggcttgaattcctgtcctgtgaaggaagccaaccagagtacgttggaaaacttcttggaaaggctaaagacgatcatgagagagaaatattcaaagtgttcgagctga

SEQ ID NO: 48 - DNA, 639 bp, Artificial sequence
  Synthetic construct
atgaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtag

SEQ ID NO: 49 - DNA, 534 bp, Artificial sequence
  Synthetic construct
atgttccatgtttcttttaggtatatctttggacttcctcccctgatccttgttctgttgccagtagcatcatctgattgtgatattgaaggtaaagatggcaaacaatatgagagtgttctaatggtcagcatcgatcaattattggacagcatgaaagaaattggtagcaattgcctgaataatgaatttaacttttttaaaagacatatctgtgatgctaataaggaaggtatgtttttattccgtgctgctcgcaagttgaggcaatttcttaaaatgaatagcactggtgattttgatctccacttattaaaagtttcagaaggcacaacaatactgttgaactgcactggccaggttaaaggaagaaaaccagctgccctgggtgaagcccaaccaacaaagagtttggaagaaaataaatctttaaaggaacagaaaaaactgaatgacttgtgtttcctaaagagactattacaagagataaaaacttgttggaataaaattttgatgggcactaaagaacactga

SEQ ID NO: 50 - DNA, 537 bp, Artificial sequence
  Synthetic construct
atgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactga

SEQ ID NO: 51 - DNA, 762 bp, Artificial sequence
  Synthetic construct
atgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 52 - DNA, 987 bp, Artificial sequence
  Synthetic construct
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttag

SEQ ID NO: 53 - DNA, 489 bp, Artificial sequence
  Synthetic construct
atgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttga

SEQ ID NO: 54 - DNA, 1401 bp, Artificial sequence
  Synthetic construct
atggtctcccaggccctcaggctcctctgccttctgcttgggcttcagggctgcctggctgcaggcggggtcgctaaggcctcaggaggagaaacacgggacatgccgtggaagccggggcctcacagagtcttcgtaacccaggaggaagcccacggcgtcctgcaccggcgccggcgcgccaacgcgttcctggaggagctgcggccgggctccctggagagggagtgcaaggaggagcagtgctccttcgaggaggcccgggagatcttcaaggacgcggagaggacgaagctgttctggatttcttacagtgatggggaccagtgtgcctcaagtccatgccagaatgggggctcctgcaaggaccagctccagtcctatatctgcttctgcctccctgccttcgagggccggaactgtgagacgcacaaggatgaccagctgatctgtgtgaacgagaacggcggctgtgagcagtactgcagtgaccacacgggcaccaagcgctcctgtcggtgccacgaggggtactctctgctggcagacggggtgtcctgcacacccacagttgaatatccatgtggaaaaatacctattctagaaaaaagaaatgccagcaaaccccaaggccgaattgtggggggcaaggtgtgccccaaaggggagtgtccatggcaggtcctgttgttggtgaatggagctcagttgtgtggggggaccctgatcaacaccatctgggtggtctccgcggcccactgtttcgacaaaatcaagaactggaggaacctgatcgcggtgctgggcgagcacgacctcagcgagcacgacggggatgagcagagccggcgggtggcgcaggtcatcatccccagcacgtacgtcccgggcaccaccaaccacgacatcgcgctgctccgcctgcaccagcccgtggtcctcactgaccatgtggtgcccctctgcctgcccgaacggacgttctctgagaggacgctggccttcgtgcgcttctcattggtcagcggctggggccagctgctggaccgtggcgccacggccctggagctcatggtcctcaacgtgccccggctgatgacccaggactgcctgcagcagtcacggaaggtgggagactccccaaatatcacggagtacatgttctgtgccggctactcggatggcagcaaggactcctgcaagggggacagtggaggcccacatgccacccactaccggggcacgtggtacctgacgggcatcgtcagctggggccagggctgcgcaaccgtgggccactttggggtgtacaccagggtctcccagtacatcgagtggctgcaaaagctcatgcgctcagagccacgcccaggagtcctcctgcgagccccatttccctag

SEQ ID NO: 55 - DNA, 4395 bp, Artificial sequence
  Synthetic construct
atgcagatagaattgtctacatgttttttcctttgcctgctcagattttgcttctccgcaactcgccgctattacctgggtgctgttgagttgagctgggattatatgcaaagtgatctcggagaactccctgtcgacgcgaggttcccaccgagggtgccaaaatcttttcccttcaatactagcgtggtctataaaaaaaccttgttcgtagagtttacggaccatctcttcaacattgccaaacctagaccaccttggatgggactgctcgggccgaccatacaggcggaagtttatgacactgttgtgattactttgaagaacatggcttctcatccggtcagtttgcatgcggtgggtgtaagttactggaaggcttctgaaggcgcggagtatgatgaccagacgtcccagcgagaaaaagaagacgataaagtttttccaggtgggagtcatacatatgtctggcaggtcctcaaggagaacggcccgatggcgtccgatccgctttgcctcacctactcctacctctcacatgttgaccttgtaaaagatttgaactcaggtttgataggtgcccttttggtgtgccgggaaggttccctcgcaaaagagaaaacgcagactcttcacaaattcatattgctgttcgcagtattcgatgaaggtaagtcctggcacagcgaaaccaagaattcactgatgcaggacagagacgctgcaagcgcccgagcctggcctaagatgcatactgttaatgggtacgttaacaggtcactccccgggttgattggttgtcataggaaatctgtatactggcatgttatagggatgggcacaacacccgaagtgcattccatcttccttgaagggcatacattcctggtacggaatcaccggcaggcctctttggaaatctctccgataacttttttgaccgctcagacccttttgatggacctcggtcagtttcttctgttctgccacatatccagccatcaacacgacgggatggaggcatatgtcaaggtcgatagctgtcccgaagagccccaacttcgcatgaagaataatgaagaagccgaagactacgacgatgatttgaccgattccgaaatggacgttgtgcgattcgacgatgacaacagcccttctttcatccaaattagatcagtggctaaaaagcacccgaaaacctgggtgcactatatcgcggcagaagaagaggattgggactacgcccccctggtcctggcgccggatgatcgaagctacaagtcccagtatctcaacaacggccctcaaaggatcggccggaagtacaagaaagttcgcttcatggcctatacagatgaaaccttcaaaaccagagaagcgattcagcatgaaagtgggattctcggtccacttctttacggggaagttggagacaccctccttattatctttaagaaccaagcgagtcggccttacaacatctatccgcatgggataaccgacgtacgcccactttactctcgaaggttgccaaagggtgttaaacatcttaaagacttcccgattctccctggcgaaatattcaaatataaatggacagtgacggtagaagatggtcccaccaaatcagacccgaggtgcttgaccagatattattcctctttcgtaaatatggagagggatctggcctctggtctgataggtccactgctgatttgctacaaggaatcagtagatcaaaggggcaaccaaataatgtccgataaacgaaacgtcatcttgtttagtgttttcgatgaaaatcggagttggtatttgacggagaacattcagcgctttctcccgaaccccgcaggggtgcaactggaggatccagaattccaggcatctaacataatgcattccataaacggctatgtctttgactctctccaattgagtgtatgtctgcatgaggtcgcatactggtatatcctctccattggggctcaaaccgatttcttgagcgtcttcttcagtggatacacatttaaacataaaatggtctatgaggataccctgactctttttcccttctctggagaaaccgtatttatgtccatggagaatcctggcttgtggatcctcgggtgtcataactctgacttccgaaaccgaggcatgacggcgctgctgaaggtttcttcttgcgataaaaacactggggattattacgaggactcatatgaggacatctccgcgtatcttcttagcaaaaataacgcgattgagcctaggagtttcagtcaaaacagccgacaccctagtcaaaacccaccagttctcaagcggcatcagcgcgagattacacgcaccacgcttcagagtgaccaggaggaaattgattatgacgacaccatcagtgtcgagatgaagaaggaggactttgatatatatgacgaagatgaaaatcagagccccagaagttttcaaaagaagacgaggcattatttcatcgcagcggttgaacggctctgggattatgggatgagcagcagtccgcacgtactcagaaacagagcgcagagcgggagcgtacctcaatttaagaaagttgtttttcaggaatttaccgacggttctttcacacagcctttgtatagaggggagctcaatgagcaccttggtttgctgggaccttatataagagccgaagttgaagataatattatggtgacgttccggaaccaagcatcccggccctacagcttctactcctctctcatctcttacgaagaagatcagcggcagggagcagaaccccgcaaaaacttcgtgaaacccaatgagacaaaaacgtatttctggaaagttcagcaccacatggcgcccaccaaggatgagtttgactgcaaggcctgggcttattttagtgatgtagatctcgaaaaagacgtgcatagtgggttgatagggcccctgttggtatgtcacacgaatactttgaacccagcccacggtcgccaggttacggtacaagagtttgcgctcttcttcacgatatttgatgagacaaagagctggtatttcacggagaatatggagagaaactgccgggcgccttgtaacatacaaatggaagatcctacgttcaaagagaactaccgattccatgctataaatgggtatataatggacacccttcccgggttggtcatggcccaagatcagcgcattcgctggtacttgcttagcatgggcagtaatgagaacatccattcaatccatttctccggtcacgttttcacggtaagaaagaaagaggagtacaaaatggctttgtacaatctgtatccgggggtgttcgaaacggtcgagatgctgcccagtaaagcggggatctggagggtcgaatgtcttatcggcgagcatttgcatgcgggcatgtctactctgttcctggtgtacagcaataaatgtcaaacgccgttgggaatggcatccgggcatatcagggacttccagataacggcttccgggcaatacgggcaatgggcaccaaagcttgcaagactccactactccgggtccataaacgcgtggtctactaaggagcctttctcctggatcaaagtagatcttctggctcccatgattatacacggcattaaaacgcaaggcgcgcgccaaaaatttagctccctctatatatcccagtttattatcatgtatagtctcgatggaaagaaatggcaaacttaccgaggtaactctactggtaccctcatggtattttttgggaacgtcgactcaagtggcattaagcataatatatttaacccgccgataatagctcgctacatacggttgcatcccactcactatagcatacgcagtacacttaggatggaactcatgggttgcgatctgaactcttgttccatgcctttggggatggagtccaaagcgatatccgacgctcaaataacagcctcaagttattttacaaacatgtttgcgacgtggagtcctagcaaggcaaggctccacctccaaggccgaagtaatgcttggcggccgcaggtgaacaaccccaaagaatggttgcaagtggatttccaaaagaccatgaaagttacgggtgtaacaacacagggcgttaaaagtttgcttacctccatgtacgttaaagagttcttgatatcatctagtcaggatgggcatcaatggacccttttctttcaaaacgggaaggtcaaagtgtttcagggcaatcaggactctttcactcccgttgtcaactccctcgacccacccctgctcaccaggtatttgagaatacatccgcagagttgggtacaccaaatcgcactcaggatggaggtgctcggctgtgaggcacaggacttgtattga

SEQ ID NO: 56 - DNA, 8441 bp, Artificial sequence
  Synthetic construct
atgattcctgccagatttgccggggtgctgcttgctctggccctcattttgccagggaccctttgtgcagaaggaactcgcggcaggtcatccacggcccgatgcagccttttcggaagtgacttcgtcaacacctttgatgggagcatgtacagctttgcgggatactgcagttacctcctggcagggggctgccagaaacgctccttctcgattattggggacttccagaatggcaagagagtgagcctctccgtgtatcttggggaattttttgacatccatttgtttgtcaatggtaccgtgacacagggggaccaaagagtctccatgccctatgcctccaaagggctgtatctagaaactgaggctgggtactacaagctgtccggtgaggcctatggctttgtggccaggatcgatggcagcggcaactttcaagtcctgctgtcagacagatacttcaacaagacctgcgggctgtgtggcaactttaacatctttgctgaagatgactttatgacccaagaagggaccttgacctcggacccttatgactttgccaactcatgggctctgagcagtggagaacagtggtgtgaacgggcatctcctcccagcagctcatgcaacatctcctctggggaaatgcagaagggcctgtgggagcagtgccagcttctgaagagcacctcggtgtttgcccgctgccaccctctggtggaccccgagccttttgtggccctgtgtgagaagactttgtgtgagtgtgctggggggctggagtgcgcctgccctgccctccggagtacgcccggacctgtgcccaggagggaatggtgctgtacggctggaccgaccacagcgcgtgcagcccagtgtgccctgctggtatggagtataggcagtgtgtgtccccttgcgccaggacctgccagagcctgcacatcaatgaaatgtgtcaggagcgatgcgtggatggctgcagctgccctgagggacagctcctggatgaaggcctctgcgtggagagcaccgagtgtccctgcgtgcattccggaaagcgctaccctcccggcacctccctctctcgagactgcaacacctgcatttgccgaaacagccagtggatctgcagcaatgaagaatgtccaggggagtgccttgtcacaggtcaatcacacttcaagagctttgacaacagatacttcaccttcagtgggatctgccagtacctgctggcccgggattgccaggaccactccttctccattgtcattgagactgtccagtgtgctgatgaccgcgacgctgtgtgcacccgctccgtcaccgtccggctgcctggcctgcacaacagccttgtgaaactgaagcatggggcaggagttgccatggatggccaggacgtccagctccccctcctgaaaggtgacctccgcatccagcatacagtgacggcctccgtgcgcctcagctacggggaggacctgcagatggactgggatggccgcgggaggctgctggtgaagctgtcccccgtctatgccgggaagacctgcggcctgtgtgggaattacaatggcaaccagggcgacgacttccttaccccctctgggctggcggagccccgggtggaggacttcgggaacgcctggaagctgcacggggactgccaggacctgcagaagcagcacagcgatccctgcgccctcaacccgcgcatgaccaggttctccgaggaggcgtgcgcggtcctgacgtcccccacattcgaggcctgccatcgtgccgtcagcccgctgccctacctgcggaactgccgctacgacgtgtgctcctgctcggacggccgcgagtgcctgtgcggcgccctggccagctatgccgcggcctgcgcggggagaggcgtgcgcgtcgcgtggcgcgagccaggccgctgtgagctgaactgcccgaaaggccaggtgtacctgcagtgcgggaccccctgcaacctgacctgccgctctctctcttacccggatgaggaatgcaatgaggcctgcctggagggctgcttctgccccccagggctctacatggatgagaggggggactgcgtgcccaaggcccagtgcccctgttactatgacggtgagatcttccagccagaagacatcttctcagaccatcacaccatgtgctactgtgaggatggcttcatgcactgtaccatgagtggagtccccggaagcttgctgcctgacgctgtcctcagcagtcccctgtctcatcgcagcaaaaggagcctatcctgtcggccccccatggtcaagctggtgtgtcccgctgacaacctgcgggctgaagggctcgagtgtaccaaaacgtgccagaactatgacctggagtgcatgagcatgggctgtgtctctggctgcctctgccccccgggcatggtccggcatgagaacagatgtgtggccctggaaaggtgtccctgcttccatcagggcaaggagtatgcccctggagaaacagtgaagattggctgcaacacttgtgtctgtcgggaccggaagtggaactgcacagaccatgtgtgtgatgccacgtgctccacgatcggcatggcccactacctcaccttcgacgggctcaaatacctgttccccggggagtgccagtacgttctggtgcaggattactgcggcagtaaccctgggacctttcggatcctagtggggaataagggatgcagccacccctcagtgaaatgcaagaaacgggtcaccatcctggtggagggaggagagattgagctgtttgacggggaggtgaatgtgaagaggcccatgaaggatgagactcactttgaggtggtggagtctggccggtacatcattctgctgctgggcaaagccctctccgtggtctgggaccgccacctgagcatctccgtggtcctgaagcagacataccaggagaaagtgtgtggcctgtgtgggaattttgatggcatccagaacaatgacctcaccagcagcaacctccaagtggaggaagaccctgtggactttgggaactcctggaaagtgagctcgcagtgtgctgacaccagaaaagtgcctctggactcatcccctgccacctgccataacaacatcatgaagcagacgatggtggattcctcctgtagaatccttaccagtgacgtcttccaggactgcaacaagctggtggaccccgagccatatctggatgtctgcatttacgacacctgctcctgtgagtccattggggactgcgcctgcttctgcgacaccattgctgcctatgcccacgtgtgtgcccagcatggcaaggtggtgacctggaggacggccacattgtgcccccagagctgcgaggagaggaatctccgggagaacgggtatgagtgtgagtggcgctataacagctgtgcacctgcctgtcaagtcacgtgtcagcaccctgagccactggcctgccctgtgcagtgtgtggagggctgccatgcccactgccctccagggaaaatcctggatgagcttttgcagacctgcgttgaccctgaagactgtccagtgtgtgaggtggctggccggcgttttgcctcaggaaagaaagtcaccttgaatcccagtgaccctgagcactgccagatttgccactgtgatgttgtcaacctcacctgtgaagcctgccaggagccgggaggcctggtggtgcctcccacagatgccccggtgagccccaccactctgtatgtggaggacatctcggaaccgccgttgcacgatttctactgcagcaggctactggacctggtcttcctgctggatggctcctccaggctgtccgaggctgagtttgaagtgctgaaggcctttgtggtggacatgatggagcggctgcgcatctcccagaagtgggtccgcgtggccgtggtggagtaccacgacggctcccacgcctacatcgggctcaaggaccggaagcgaccgtcagagctgcggcgcattgccagccaggtgaagtatgcgggcagccaggtggcctccaccagcgaggtcttgaaatacacactgttccaaatcttcagcaagatcgaccgccctgaagcctcccgcatcaccctgctcctgatggccagccaggagccccaacggatgtcccggaactttgtccgctacgtccagggcctgaagaagaagaaggtcattgtgatcccggtgggcattgggccccatgccaacctcaagcagatccgcctcatcgagaagcaggcccctgagaacaaggccttcgtgctgagcagtgtggatgagctggagcagcaaagggacgagatcgttagctacctctgtgaccttgcccctgaagcccctcctcctactctgccccccgacatggcacaagtcactgtgggcccggggctcttgggggtttcgaccctggggcccaagaggaactccatggttctggatgtggcgttcgtcctggaaggatcggacaaaattggtgaagccgacttcaacaggagcaaggagttcatggaggaggtgattcagcggatggatgtgggccaggacagcatccacgtcacggtgctgcagtactcctacatggtgactgtggagtaccccttcagcgaggcacagtccaaaggggacatcctgcagcgggtgcgagagatccgctaccagggcggcaacaggaccaacactgggctggccctgcggtacctctctgaccacagcttcttggtcagccagggtgaccgggagcaggcgcccaacctggtctacatggtcaccggaaatcctgcctctgatgagatcaagaggctgcctggagacatccaggtggtgcccattggagtgggccctaatgccaacgtgcaggagctggagaggattggctggcccaatgcccctatcctcatccaggactttgagacgctcccccgagaggctcctgacctggtgctgcagaggtgctgctccggagaggggctgcagatccccaccctctcccctgcacctgactgcagccagcccctggacgtgatccttctcctggatggctcctccagtttcccagcttcttattttgatgaaatgaagagtttcgccaaggctttcatttcaaaagccaatatagggcctcgtctcactcaggtgtcagtgctgcagtatggaagcatcaccaccattgacgtgccatggaacgtggtcccggagaaagcccatttgctgagccttgtggacgtcatgcagcgggagggaggccccagccaaatcggggatgccttgggctttgctgtgcgatacttgacttcagaaatgcatggtgccaggccgggagcctcaaaggcggtggtcatcctggtcacggacgtctctgtggattcagtggatgcagcagctgatgccgccaggtccaacagagtgacagtgttccctattggaattggagatcgctacgatgcagcccagctacggatcttggcaggcccagcaggcgactccaacgtggtgaagctccagcgaatcgaagacctccctaccatggtcaccttgggcaattccttcctccacaaactgtgctctggatttgttaggatttgcatggatgaggatgggaatgagaagaggcccggggacgtctggaccttgccagaccagtgccacaccgtgacttgccagccagatggccagaccttgctgaagagtcatcgggtcaactgtgaccgggggctgaggccttcgtgccctaacagccagtcccctgttaaagtggaagagacctgtggctgccgctggacctgcccctgcgtgtgcacaggcagctccactcggcacatcgtgacctttgatgggcagaatttcaagctgactggcagctgttcttatgtcctatttcaaaacaaggagcaggacctggaggtgattctccataatggtgcctgcagccctggagcaaggcagggctgcatgaaatccatcgaggtgaagcacagtgccctctccgtcgagctgcacagtgacatggaggtgacggtgaatgggagactggtctctgttccttacgtgggtgggaacatggaagtcaacgtttatggtgccatcatgcatgaggtcagattcaatcaccttggtcacatcttcacattcactccacaaaacaatgagttccaactgcagctcagccccaagacttttgcttcaaagacgtatggtctgtgtgggatctgtgatgagaacggagccaatgacttcatgctgagggatggcacagtcaccacagactggaaaacacttgttcaggaatggactgtgcagcggccagggcagacgtgccagcccatcctggaggagcagtgtcttgtccccgacagctcccactgccaggtcctcctcttaccactgtttgctgaatgccacaaggtcctggctccagccacattctatgccatctgccagcaggacagttgccaccaggagcaagtgtgtgaggtgatcgcctcttatgcccacctctgtcggaccaacggggtctgcgttgactggaggacacctgatttctgtgctatgtcatgcccaccatctctggtctacaaccactgtgagcatggctgtccccggcactgtgatggcaacgtgagctcctgtggggaccatccctccgaaggctgtttctgccctccagataaagtcatgttggaaggcagctgtgtccctgaagaggcctgcactcagtgcattggtgaggatggagtccagcaccagttcctggaagcctgggtcccggaccaccagccctgtcagatctgcacatgcctcagcgggcggaaggtcaactgcacaacgcagccctgccccacggccaaagctcccacgtgtggcctgtgtgaagtagcccgcctccgccagaatgcagaccagtgctgccccgagtatgagtgtgtgtgtgacccagtgagctgtgacctgcccccagtgcctcactgtgaacgtggcctccagcccacactgaccaaccctggcgagtgcagacccaacttcacctgcgcctgcaggaaggaggagtgcaaaagagtgtccccaccctcctgccccccgcaccgtttgcccacccttcggaagacccagtgctgtgatgagtatgagtgtgcctgcaactgtgtcaactccacagtgagctgtccccttgggtacttggcctcaactgccaccaatgactgtggctgtaccacaaccacctgccttcccgacaaggtgtgtgtccaccgaagcaccatctaccctgtgggccagttctgggaggagggctgcgatgtgtgcacctgcaccgacatggaggatgccgtgatgggcctccgcgtggcccagtgctcccagaagccctgtgaggacagctgtcggtcgggcttcacttacgttctgcatgaaggcgagtgctgtggaaggtgcctgccatctgcctgtgaggtggtgactggctcaccgcggggggactcccagtcttcctggaagagtgtcggctcccagtgggcctccccggagaacccctgcctcatcaatgagtgtgtccgagtgaaggaggaggtctttatacaacaaaggaacgtctcctgcccccagctggaggtccctgtctgcccctcgggctttcagctgagctgtaagacctcagcgtgctgcccaagctgtcgctgtgagcgcatggaggcctgcatgctcaatggcactgtcattgggcccgggaagactgtgatgatcgatgtgtgcacgacctgccgctgcatggtgcaggtgggggtcatctctggattcaagctggagtgcaggaagaccacctgcaacccctgccccctgggttacaaggaagaaaataacacaggtgaatgttgtgggagatgtttgcctacggcttgcaccattcagctaagaggaggacagatcatgacactgaagcgtgatgagacgctccaggatggctgtgatactcacttctgcaaggtcaatgagagaggagagtacttctgggagaagagggtcacaggctgcccaccctttgatgaacacaagtgtctggctgagggaggtaaaattatgaaaattccaggcacctgctgtgacacatgtgaggagcctgagtgcaacgacatcactgccaggctgcagtatgtcaaggtgggaagctgtaagtctgaagtagaggtggatatccactactgccagggcaaatgtgccagcaaagccatgtactccattgacatcaacgatgtgcaggaccagtgctcctgctgctctccgacacggacggagcccatgcaggtggccctgcactgcaccaatggctctgttgtgtaccatgaggttctcaatgccatggagtgcaaatgctcccccaggaagtgcagcaagtga

SEQ ID NO: 57 - DNA, 543 bp, Artificial sequence
  Synthetic construct
atgaaaagcatttactttgtggctggattatttgtaatgctggtacaaggcagctggcaacgttcccttcaagacacagaggagaaatccagatcattctcagcttcccaggcagacccactcagtgatcctgatcagatgaacgaggacaagcgccattcacagggcacattcaccagtgactacagcaagtatctggactccaggcgtgcccaagattttgtgcagtggttgatgaataccaagaggaacaggaataacattgccaaacgtcacgatgaatttgagagacatgctgaagggacctttaccagtgatgtaagttcttatttggaaggccaagctgccaaggaattcattgcttggctggtgaaaggccgaggaaggcgagatttcccagaagaggtcgccattgttgaagaacttggccgcagacatgctgatggttctttctctgatgagatgaacaccattcttgataatcttgccgccagggactttataaactggttgattcagaccaaaatcactgacaggaaataa

SEQ ID NO: 58 - DNA, 1386 bp, Artificial sequence
  Synthetic construct
atgcagcgcgtgaacatgatcatggcagaatcaccaggcctcatcaccatctgccttttaggatatctactcagtgctgaatgtacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaa

SEQ ID NO: 59 - DNA, 1251 bp, Artificial sequence
  Synthetic construct
gggacggcgaggcaggcgctcagagccccgcagcctggcccgtgaccccgcagagacgctgaggaccgcgacgagtcgtgtctgaacccggcttttccaattggcctgctccatccgaacagcgtcaactccatggcgcggttcctgacactttgcacttggctgctgttgctcggccccgggctcctggcgaccgtgcgggccgaatgcagccaggattgcgcgacgtgcagctaccgcctagtgcgcccggccgacatcaacttcctggcttgcgtaatggaatgtgaaggtaaactgccttctctgaaaatttgggaaacctgcaaggagctcctgcagctgtccaaaccagagcttcctcaagatggcaccagcaccctcagagaaaatagcaaaccggaagaaagccatttgctagccaaaaggtatgggggcttcatgaaaaggtatggaggcttcatgaagaaaatggatgagctttatcccatggagccagaagaagaggccaatggaagtgagatcctcgccaagcggtatgggggcttcatgaagaaggatgcagaggaggacgactcgctggccaattcctcagacctgctaaaagagcttctggaaacaggggacaaccgagagcgtagccaccaccaggatggcagtgataatgaggaagaagtgagcaagagatatgggggcttcatgagaggcttaaagagaagcccccaactggaagatgaagccaaagagctgcagaagcgatatgggggcttcatgagaagagtaggtcgcccagagtggtggatggactaccagaaacggtatggaggtttcctgaagcgctttgccgaggctctgccctccgacgaagaaggcgaaagttactccaaagaagttcctgaaatggaaaaaagatacggaggatttatgagattttaatatcttttcccactagtggccccaggccccagcaagcctccctccatcctccagtgggaaactgttgatggtgttttattgtcatgtgttgcttgccttgtatagttgacttcattgtctggataactatacaacctgaaaactgtcatttcaggttctgtgctctttttggagtctttaagctcagtattagtctattgcagctatctcgttttcatgctaaaatagtttttgttatcttgtctcttatttttgacaaacatcaataaatgcttacttgtatatagagataataaacctattaccccaagtgca

SEQ ID NO: 60 - DNA, 3624 bp, Artificial sequence
  Synthetic construct
atgctgctcactcttatcattctgttgccagtagtttcaaaatttagttttgttagtctctcagcaccgcagcactggagctgtcctgaaggtactctcgcaggaaatgggaattctacttgtgtgggtcctgcacccttcttaattttctcccatggaaatagtatctttaggattgacacagaaggaaccaattatgagcaattggtggtggatgctggtgtctcagtgatcatggattttcattataatgagaaaagaatctattgggtggatttagaaagacaacttttgcaaagagtttttctgaatgggtcaaggcaagagagagtatgtaatatagagaaaaatgtttctggaatggcaataaattggataaatgaagaagttatttggtcaaatcaacaggaaggaatcattacagtaacagatatgaaaggaaataattcccacattcttttaagtgctttaaaatatcctgcaaatgtagcagttgatccagtagaaaggtttatattttggtcttcagaggtggctggaagcctttatagagcagatctcgatggtgtgggagtgaaggctctgttggagacatcagagaaaataacagctgtgtcattggatgtgcttgataagcggctgttttggattcagtacaacagagaaggaagcaattctcttatttgctcctgtgattatgatggaggttctgtccacattagtaaacatccaacacagcataatttgtttgcaatgtccctttttggtgaccgtatcttctattcaacatggaaaatgaagacaatttggatagccaacaaacacactggaaaggacatggttagaattaacctccattcatcatttgtaccacttggtgaactgaaagtagtgcatccacttgcacaacccaaggcagaagatgacacttgggagcctgagcagaaactttgcaaattgaggaaaggaaactgcagcagcactgtgtgtgggcaagacctccagtcacacttgtgcatgtgtgcagagggatacgccctaagtcgagaccggaagtactgtgaagatgttaatgaatgtgctttttggaatcatggctgtactcttgggtgtaaaaacacccctggatcctattactgcacgtgccctgtaggatttgttctgcttcctgatgggaaacgatgtcatcaacttgtttcctgtccacgcaatgtgtctgaatgcagccatgactgtgttctgacatcagaaggtcccttatgtttctgtcctgaaggctcagtgcttgagaaagatgggaaaacatgtagcggttgttcctcacccgataatggtggatgtagccagctctgcgttcctcttagcccagtatcctgggaatgtgattgctttcctgggtatgacctacaactggatgaaaaaagctgtgcagcttcaggaccacaaccatttttgctgtttgccaattctcaagatattcgacacatgcattttgatggaacagactatggaactctgctcagccagcagatgggaatggtttatgccctagatcatgaccctgtggaaaataagatatactttgcccatacagccctgaagtggatagagagagctaatatggatggttcccagcgagaaaggcttattgaggaaggagtagatgtgccagaaggtcttgctgtggactggattggccgtagattctattggacagacagagggaaatctctgattggaaggagtgatttaaatgggaaacgttccaaaataatcactaaggagaacatctctcaaccacgaggaattgctgttcatccaatggccaagagattattctggactgatacagggattaatccacgaattgaaagttcttccctccaaggccttggccgtctggttatagccagctctgatctaatctggcccagtggaataacgattgacttcttaactgacaagttgtactggtgcgatgccaagcagtctgtgattgaaatggccaatctggatggttcaaaacgccgaagacttacccagaatgatgtaggtcacccatttgctgtagcagtgtttgaggattatgtgtggttctcagattgggctatgccatcagtaataagagtaaacaagaggactggcaaagatagagtacgtctccaaggcagcatgctgaagccctcatcactggttgtggttcatccattggcaaaaccaggagcagatccctgcttatatcaaaacggaggctgtgaacatatttgcaaaaagaggcttggaactgcttggtgttcgtgtcgtgaaggttttatgaaagcctcagatgggaaaacgtgtctggctctggttggtcatcagctgttggcaggtggtgaagttgatctaaagaaccaagtaacaccattggacatcttgtccaagactagagtgtcagaagataacattacagaatctcaacacatgctagtggctgaaatcatggtgtcagatcaagatgactgtgctcctgtgggatgcagcatgtatgctcggtgtatttcagagggagaggatgccacatgtcagtgtttgaaaggatttgctggggatggaaaactatgttctgatatagatgaatgtgagatgggtgtcccagtgtgcccccctgcctcctccaagtgcatcaacaccgaaggtggttatgtctgccggtgctcagaaggctaccaaggagatgggattcactgtcttgatattgatgagtgccaactgggggtgcacagctgtggagagaatgccagctgcacaaatacagagggaggctatacctgcatgtgtgctggacgcctgtctgaaccaggactgatttgccctgactctactccaccccctcacctcagggaagatgaccaccactattccgtaagaaatagtgactctgaatgtcccctgtcccacgatgggtactgcctccatgatggtgtgtgcatgtatattgaagcattggacaagtatgcatgcaactgtgttgttggctacatcggggagcgatgtcagtaccgagacctgaagtggtgggaactgcgccacgctggccacgggcagcagcagaaggtcatcgtggtggctgtctgcgtggtggtgcttgtcatgctgctcctcctgagcctgtggggggcccactactacaggactcagaagctgctatcgaaaaacccaaagaatccttatgaggagtcgagcagagatgtgaggagtcgcaggcctgctgacactgaggatgggatgtcctcttgccctcaaccttggtttgtggttataaaagaacaccaagacctcaagaatgggggtcaaccagtggctggtgaggatggccaggcagcagatgggtcaatgcaaccaacttcatggaggcaggagccccagttatgtggaatgggcacagagcaaggctgctggattccagtatccagtgataagggctcctgtccccaggtaatggagcgaagctttcatatgccctcctatgggacacagacccttgaagggggtgtcgagaagccccattctctcctatcagctaacccattatggcaacaaagggccctggacccaccacaccaaatggagctgactcagtga

SEQ ID NO: 61 - DNA, 462 bp, Artificial sequence
  Synthetic construct
atgggaaaaatcagcagtcttccaacccaattatttaagtgctgcttttgtgatttcttgaaggtgaagatgcacaccatgtcctcctcgcatctcttctacctggcgctgtgcctgctcaccttcaccagctctgccacggctggaccggagacgctctgcggggctgagctggtggatgctcttcagttcgtgtgtggagacaggggcttttatttcaacaagcccacagggtatggctccagcagtcggagggcgcctcagacaggcatcgtggatgagtgctgcttccggagctgtgatctaaggaggctggagatgtattgcgcacccctcaagcctgccaagtcagctcgctctgtccgtgcccagcgccacaccgacatgcccaagacccagaaggaagtacatttgaagaacgcaagtagagggagtgcaggaaacaagaactacaggatgtag

SEQ ID NO: 62 - DNA, 1170 bp, Artificial sequence
  Synthetic construct
atgccgccctccgggctgcggctgctgctgctgctgctaccgctgctgtggctactggtgctgacgcctggccggccggccgcgggactatccacctgcaagactatcgacatggagctggtgaagcggaagcgcatcgaggccatccgcggccagatcctgtccaagctgcggctcgccagccccccgagccagggggaggtgccgcccggcccgctgcccgaggccgtgctcgccctgtacaacagcacccgcgaccgggtggccggggagagtgcagaaccggagcccgagcctgaggccgactactacgccaaggaggtcacccgcgtgctaatggtggaaacccacaacgaaatctatgacaagttcaagcagagtacacacagcatatatatgttcttcaacacatcagagctccgagaagcggtacctgaacccgtgttgctctcccgggcagagctgcgtctgctgaggctcaagttaaaagtggagcagcacgtggagctgtaccagaaatacagcaacaattcctggcgatacctcagcaaccggctgctggcacccagcgactcgccagagtggttatcttttgatgtcaccggagttgtgcggcagtggttgagccgtggaggggaaattgagggctttcgccttagcgcccactgctcctgtgacagcagggataacacactgcaagtggacatcaacgggttcactaccggccgccgaggtgacctggccaccattcatggcatgaaccggcctttcctgcttctcatggccaccccgctggagagggcccagcatctgcaaagctcccggcaccgccgagccctggacaccaactattgcttcagctccacggagaagaactgctgcgtgcggcagctgtacattgacttccgcaaggacctcggctggaagtggatccacgagcccaagggctaccatgccaacttctgcctcgggccctgcccctacatttggagcctggacacgcagtacagcaaggtcctggccctgtacaaccagcataacccgggcgcctcggcggcgccgtgctgcgtgccgcaggcgctggagccgctgcccatcgtgtactacgtgggccgcaagcccaaggtggagcagctgtccaacatgatcgtgcgctcctgcaagtgcagc

SEQ ID NO: 63 - DNA, 577 bp, Artificial sequence
  Synthetic construct
actcttctggtccccacagactcagagagaacccaccatggtgctgtctcctgccgacaagaccaacgtcaaggccgcctggggtaaggtcggcgcgcacgctggcgagtatggtgcggaggccctggagaggatgttcctgtccttccccaccaccaagacctacttcccgcacttcgacctgagccacggctctgcccaggttaagggccacggcaagaaggtggccgacgcgctgaccaacgccgtggcgcacgtggacgacatgcccaacgcgctgtccgccctgagcgacctgcacgcgcacaagcttcgggtggacccggtcaacttcaagctcctaagccactgcctgctggtgaccctggccgcccacctccccgccgagttcacccctgcggtgcacgcctccctggacaagttcctggcttctgtgagcaccgtgctgacctccaaataccgttaagctggagcctcggtggccatgcttcttgccccttgggcctccccccagcccctcctccccttcctgcacccgtacccccgtggtctttgaataaagtctgagtgggcggca

SEQ ID NO: 64 - DNA, 628 bp, Artificial sequence
  Synthetic construct
acatttgcttctgacacaactgtgttcactagcaacctcaaacagacaccatggtgcatctgactcctgaggagaagtctgccgttactgccctgtggggcaaggtgaacgtggatgaagttggtggtgaggccctgggcaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcaggctcctgggcaacgtgctggtctgtgtgctggcccatcactttggcaaagaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 65 - DNA, 6781 bp, Artificial sequence
  Synthetic construct
cggccccagaaaacccgagcgagtagggggcggcgcgcaggagggaggagaactgggggcgcgggaggctggtgggtgtggggggtggagatgtagaagatgtgacgccgcggcccggcgggtgccagattagcggacgcggtgcccgcggttgcaacgggatcccgggcgctgcagcttgggaggcggctctccccaggcggcgtccgcggagacacccatccgtgaaccccaggtcccgggccgccggctcgccgcgcaccaggggccggcggacagaagagcggccgagcggctcgaggctgggggaccgcgggcgcggccgcgcgctgccgggcgggaggctggggggccggggccggggccgtgccccggagcgggtcggaggccggggccggggccgggggacggcggctccccgcgcggctccagcggctcggggatcccggccgggccccgcagggaccatggcagccgggagcatcaccacgctgcccgccttgcccgaggatggcggcagcggcgccttcccgcccggccacttcaaggaccccaagcggctgtactgcaaaaacgggggcttcttcctgcgcatccaccccgacggccgagttgacggggtccgggagaagagcgaccctcacatcaagctacaacttcaagcagaagagagaggagttgtgtctatcaaaggagtgtgtgctaaccgttacctggctatgaaggaagatggaagattactggcttctaaatgtgttacggatgagtgtttcttttttgaacgattggaatctaataactacaatacttaccggtcaaggaaatacaccagttggtatgtggcactgaaacgaactgggcagtataaacttggatccaaaacaggacctgggcagaaagctatactttttcttccaatgtctgctaagagctgattttaatggccacatctaatctcatttcacatgaaagaagaagtatattttagaaatttgttaatgagagtaaaagaaaataaatgtgtatagctcagtttggataattggtcaaacaattttttatccagtagtaaaatatgtaaccattgtcccagtaaagaaaaataacaaaagttgtaaaatgtatattctcccttttatattgcatctgctgttacccagtgaagcttacctagagcaatgatctttttcacgcatttgctttattcgaaaagaggcttttaaaatgtgcatgtttagaaacaaaatttcttcatggaaatcatatacattagaaaatcacagtcagatgtttaatcaatccaaaatgtccactatttcttatgtcattcgttagtctacatgtttctaaacatataaatgtgaatttaatcaattcctttcatagttttataattctctggcagttccttatgatagagtttataaaacagtcctgtgtaaactgctggaagttcttccacagtcaggtcaattttgtcaaacccttctctgtacccatacagcagcagcctagcaactctgctggtgatgggagttgtattttcagtcttcgccaggtcattgagatccatccactcacatcttaagcattcttcctggcaaaaatttatggtgaatgaatatggctttaggcggcagatgatatacatatctgacttcccaaaagctccaggatttgtgtgctgttgccgaatactcaggacggacctgaattctgattttataccagtctcttcaaaaacttctcgaaccgctgtgtctcctacgtaaaaaaagagatgtacaaatcaataataattacacttttagaaactgtatcatcaaagattttcagttaaagtagcattatgtaaaggctcaaaacattaccctaacaaagtaaagttttcaatacaaattctttgccttgtggatatcaagaaatcccaaaatattttcttaccactgtaaattcaagaagcttttgaaatgctgaatatttctttggctgctacttggaggcttatctacctgtacatttttggggtcagctctttttaacttcttgctgctctttttcccaaaaggtaaaaatatagattgaaaagttaaaacattttgcatggctgcagttcctttgtttcttgagataagattccaaagaacttagattcatttcttcaacaccgaaatgctggaggtgtttgatcagttttcaagaaacttggaatataaataattttataattcaacaaaggttttcacattttataaggttgatttttcaattaaatgcaaatttgtgtggcaggatttttattgccattaacatatttttgtggctgctttttctacacatccagatggtccctctaactgggctttctctaattttgtgatgttctgtcattgtctcccaaagtatttaggagaagccctttaaaaagctgccttcctctaccactttgctggaaagcttcacaattgtcacagacaaagatttttgttccaatactcgttttgcctctatttttcttgtttgtcaaatagtaaatgatatttgcccttgcagtaattctactggtgaaaaacatgcaaagaagaggaagtcacagaaacatgtctcaattcccatgtgctgtgactgtagactgtcttaccatagactgtcttacccatcccctggatatgctcttgttttttccctctaatagctatggaaagatgcatagaaagagtataatgttttaaaacataaggcatttgtctgccatttttcaattacatgctgacttcccttacaattgagatttgcccataggttaaacatggttagaaacaactgaaagcataaaagaaaaatctaggccgggtgcagtggctcatgcctatattccctgcactttgggaggccaaagcaggaggatcgcttgagcccaggagttcaagaccaacctggtgaaaccccgtctctacaaaaaaacacaaaaaatagccaggcatggtggcgtgtacatgtggtctcagatacttgggaggctgaggtgggagggttgatcacttgaggctgagaggtcaaggttgcagtgagccataatcgtgccactgcagtccagcctaggcaacagagtgagactttgtctcaaaaaaagagaaattttccttaataagaaaagtaatttttactctgatgtgcaatacatttgttattaaatttattatttaagatggtagcactagtcttaaattgtataaaatatcccctaacatgtttaaatgtccatttttattcattatgctttgaaaaataattatggggaaatacatgtttgttattaaatttattattaaagatagtagcactagtcttaaatttgatataacatctcctaacttgtttaaatgtccatttttattctttatgtttgaaaataaattatggggatcctatttagctcttagtaccactaatcaaaagttcggcatgtagctcatgatctatgctgtttctatgtcgtggaagcactggatgggggtagtgagcaaatctgccctgctcagcagtcaccatagcagctgactgaaaatcagcactgcctgagtagttttgatcagtttaacttgaatcactaactgactgaaaattgaatgggcaaataagtgcttttgtctccagagtatgcgggagacccttccacctcaagatggatatttcttccccaaggatttcaagatgaattgaaatttttaatcaagatagtgtgctttattctgttgtattttttattattttaatatactgtaagccaaactgaaataacatttgctgttttataggtttgaagaacataggaaaaactaagaggttttgtttttatttttgctgatgaagagatatgtttaaatatgttgtattgttttgtttagttacaggacaataatgaaatggagtttatatttgttatttctattttgttatatttaataatagaattagattgaaataaaatataatgggaaataatctgcagaatgtgggttttcctggtgtttccctctgactctagtgcactgatgatctctgataaggctcagctgctttatagttctctggctaatgcagcagatactcttcctgccagtggtaatacgattttttaagaaggcagtttgtcaattttaatcttgtggatacctttatactcttagggtattattttatacaaaagccttgaggattgcattctattttctatatgaccctcttgatatttaaaaaacactatggataacaattcttcatttacctagtattatgaaagaatgaaggagttcaaacaaatgtgtttcccagttaactagggtttactgtttgagccaatataaatgtttaactgtttgtgatggcagtattcctaaagtacattgcatgttttcctaaatacagagtttaaataatttcagtaattcttagatgattcagcttcatcattaagaatatcttttgttttatgttgagttagaaatgccttcatatagacatagtctttcagacctctactgtcagttttcatttctagctgctttcagggttttatgaattttcaggcaaagctttaatttacactaagcttaggaagtatggctaatgccaacggcagtttttttcttcttaattccacatgactgaggcatatatgatctctgggtaggtgagttgttgtgacaaccacaagcactttttttttttttaaagaaaaaaaggtagtgaatttttaatcatctggactttaagaaggattctggagtatacttaggcctgaaattatatatatttggcttggaaatgtgtttttcttcaattacatctacaagtaagtacagctgaaattcagaggacccataagagttcacatgaaaaaaatcaatttatttgaaaaggcaagatgcaggagagaggaagccttgcaaacctgcagactgctttttgcccaatatagattgggtaaggctgcaaaacataagcttaattagctcacatgctctgctctcacgtggcaccagtggatagtgtgagagaattaggctgtagaacaaatggccttctctttcagcattcacaccactacaaaatcatcttttatatcaacagaagaataagcataaactaagcaaaaggtcaataagtacctgaaaccaagattggctagagatatatcttaatgcaatccattttctgatggattgttacgagttggctatataatgtatgtatggtattttgatttgtgtaaaagttttaaaaatcaagctttaagtacatggacatttttaaataaaatatttaaagacaatttagaaaattgccttaatatcattgttggctaaatagaataggggacatgcatattaaggaaaaggtcatggagaaataatattggtatcaaacaaatacattgatttgtcatgatacacattgaatttgatccaatagtttaaggaataggtaggaaaatttggtttctatttttcgatttcctgtaaatcagtgacataaataattcttagcttattttatatttccttgtcttaaatactgagctcagtaagttgtgttaggggattatttctcagttgagactttcttatatgacattttactatgttttgactacctgactattaaaaataaatagtagatacaattttcataaagtgaagaattatataatcactgctttataactgactttattatatttatttcaaagttcatttaaaggctactattcatcctctgtgatggaatggtcaggaatttgttttctcatagtttaattccaacaacaatattagtcgtatccaaaataacctttaatgctaaactttactgatgtatatccaaagcttctcattttcagacagattaatccagaagcagtcataaacagaagaataggtggtatgttcctaatgatattatttctactaatggaataaactgtaatattagaaattatgctgctaattatatcagctctgaggtaatttctgaaatgttcagactcagtcggaacaaattggaaaatttaaatttttattcttagctataaagcaagaaagtaaacacattaatttcctcaacatttttaagccaattaaaaatataaaagatacacaccaatatcttcttcaggctctgacaggcctcctggaaacttccacatatttttcaactgcagtataaagtcagaaaataaagttaacataactttcactaacacacacatatgtagatttcacaaaatccacctataattggtcaaagtggttgagaatatattttttagtaattgcatgcaaaatttttctagcttccatcctttctccctcgtttcttctttttttgggggagctggtaactgatgaaatcttttcccaccttttctcttcaggaaatataagtggttttgtttggttaacgtgatacattctgtatgaatgaaacattggagggaaacatctactgaatttctgtaatttaaaatattttgctgctagttaactatgaacagatagaagaatcttacagatgctgctataaataagtagaaaatataaatttcatcactaaaatatgctattttaaaatctatttcctatattgtatttctaatcagatgtattactcttattatttctattgtatgtgttaatgattttatgtaaaaatgtaattgcttttcatgagtagtatgaataaaattgattagtttgtgttttcttgtctcccaaaaaaa

(19) United States
(12) Patent Application Publication
(10) Pub. No.: US 2022/0411795 A1
(43) Pub. Date: Dec. 29, 2022

(54) ENGINEERED CELLS FOR CONTROLLED PRODUCTION

(71) Applicant: William Marsh Rice University, Houston, TX (US)

(72) Inventors: Omid VEISEH, Houston, TX (US); Isaac HILTON, Houston, TX (US); Jing LI, Houston, TX (US); Maria Isabel JARVIS RUOCCO, Houston, TX (US); Christian Cody SCHREIB, Houston, TX (US)

(73) Assignee: William Marsh Rice University, Houston, TX (US)

(21) Appl. No.: 17/773,572

(22) PCT Filed: Oct. 30, 2020
(86) PCT No.: PCT/US2020/058166

§ 371 Date: Apr. 29, 2022

Related U.S. Application Data
(60) Provisional application No. 62/929,006, filed on Oct. 31, 2019.

(51) Int. Cl. C12N 15/113; C07K 14/475; C07K 14/54; C07K 14/755; C07K 14/805; C12N 9/22; C12N 9/64

(52) U.S. Cl. CPC C12N 15/113; C07K 14/475; C07K 14/54; C07K 14/755; C07K 14/805; C12N 9/22; C12N 9/6437; C12N 9/644; C12N 2310/20; C12N 2740/16043; C12N 2830/002

(57) ABSTRACT

The present disclosure provides expression constructs designed to provide for stable and/or inducible, tightly controlled production of genetically encoded payloads from engineered cells. These cassettes allow cells to be engineered to express genetically encoded payloads despite epigenetic silencing. As such, provided herein are expression systems for use in methods to engineer cells using CRISPR dCas9-activator systems such that expression of genetically encoded payloads (e.g., therapeutic proteins) can be optimized to overcome epigenetic silencing. In addition, provided herein are engineered cells comprising the expression systems.

### REFERENCE TO RELATED APPLICATIONS

The present application claims the priority benefit of U.S. provisional application No. 62/929,006, filed Oct. 31, 2019, the entire contents of which is incorporated herein by reference.

### REFERENCE TO A SEQUENCE LISTING

The instant application contains a Sequence Listing, which has been submitted in ASCII format via EFS-Web and is hereby incorporated by reference in its entirety. Said ASCII copy, created on Oct. 19, 2020, is named RICEP0067WO_ST25.txt and is 120 kilobytes in size.

### BACKGROUND

### 1. Field

The present invention relates generally to the fields of biology and medicine. More particularly, it concerns promoter—dCas9-activator feedback and induction loops for tightly controlled production of genetically encoded payloads.

### 2. Description of Related Art

The efficacy of cell-based therapeutics relies, in large part, on the expression yield of a therapeutic gene. Current cell-based approaches are susceptible to problems in expression, especially over time, resulting from gradual epigenetic silencing of the promoter responsible for expression of the therapeutic gene. Durability of expression and overcoming epigenetic silencing are challenges. New engineered cassette architectures are needed to address these obstacles.

### SUMMARY

As such, provided herein are expression systems for use in methods to engineer cells using CRISPR dCas9-activator systems such that expression of genetically encoded payloads (e.g., therapeutic proteins) can be optimized to overcome epigenetic silencing. In addition, provided herein are engineered cells comprising the expression systems.

In one embodiment, provided herein are nucleic acid compositions comprising (a) a first expression cassette comprising a pol II promoter operably linked to a coding sequence for a therapeutic protein, wherein the pol II promoter is, e.g., a constitutive promoter; (b) a second expression cassette comprising a pol II promoter operably linked to a coding sequence of a dCas9-activator protein, e.g., wherein the pol II promoter is the same promoter that is present in the first expression cassette; and (c) a third expression cassette comprising a pol III promoter operably linked to a coding sequence for a guide RNA (gRNA), wherein the gRNA is able to hybridize to the pol II promoters that are present in the first and second expression cassettes.

In some aspects, each expression cassette is located on a separate nucleic acid molecule. In some aspects, the nucleic acid molecules on which the first, the second, and the third expression cassettes are located each comprise an antibiotic resistance gene. In some aspects, the antibiotic resistance genes on the first, the second, and the third expression cassettes are the same. In some aspects, the antibiotic resistance genes on the first, the second, and the third expression cassettes are different. In some aspects, the antibiotic resistance genes each, independently, encode a protein that confers resistance to an antibiotic selected from neomycin, puromycin, blasticidin, zeocin, and hygromycin. The sequence of the protein encoded by the antibiotic resistance gene may be as depicted in Table 1. In some aspects, the antibiotic resistance gene on one or more of the nucleic acid molecules is flanked by a loxP site.

In some aspects, at least two of the expression cassettes are located on a single nucleic acid molecule. In some aspects, all three expression cassettes are located on a single nucleic acid molecule. In some aspects, the nucleic acid molecule further comprises an antibiotic resistance gene. In some aspects, the antibiotic resistance gene encodes a protein that confers resistance to an antibiotic selected from neomycin, puromycin, blasticidin, zeocin, and hygromycin. In some aspects, the antibiotic resistance gene is flanked by a loxP site.

In some aspects, the pol II promoter is a cell-type specific promoter. In some aspects, the pol II promoter is an EF-1α promoter, a CMV promoter, a Ubc promoter, a PGK promoter, a VMD2 promoter, or a CAG promoter. In some aspects, the pol III promoter is a U6 promoter. The sequence of the promoter may be as depicted in Table 1.

In some aspects, the first and/or second and/or third expression cassette comprises an enhancer element. In some aspects, the first and/or second expression cassette comprises an intron, a poly A signal, or a combination thereof. In some aspects, the first and/or second and/or third expression cassette comprises a filler polynucleotide.

In some aspects, the therapeutic protein is human IL-2, VEGF alpha, TGF beta, IL-4, IL-6, IL-7, IL-10, IL-12a, IL-12b, IL-15, Factor VII, Factor VIII, VWF, GCG, Factor IX, proenkephalin, EGF, IGF-1, TGF-beta1, hemoglobin (alpha-globin), hemoglobin (beta-globin), or bFGF. The sequence of the therapeutic protein may be as depicted in Table 1.

In some aspects, the first and/or second and/or third expression cassette is comprised in a viral vector. In some aspects, the viral vector is selected from an adeno-associated viral (AAV) vector, a lentiviral vector, or a retroviral vector.

In one embodiment, provided herein are cells comprising the nucleic acid composition of any one of the present embodiments. In some aspects, the expression cassettes are integrated into the genome of the cell. In some aspects, the cells express the therapeutic protein. In some aspects, the expression level of the therapeutic protein is stable over time. In some aspects, the pol II promoter drives a high level of transcription in that cell, e.g., relative to a reference level.

In one embodiment, provided herein are pharmaceutical compositions comprising the cells of any one of the present embodiments and a pharmaceutically acceptable carrier.

In one embodiment, provided herein are bioreactors comprising the cells of any one of the present embodiments.

In one embodiment, provided herein are methods of producing a therapeutic protein, the methods comprising culturing the cells of any one of the present embodiments under conditions to allow for expression of the therapeutic protein and isolating the therapeutic protein from the cells.

In one embodiment, provided herein are recombinant adeno-associated virus (rAAV) vectors comprising an AAV capsid protein and the nucleic acid composition of any one of the present embodiments. In some aspects, the AAV vector comprises an AAV particle comprising AAV capsid proteins, and wherein the first and/or second and/or third expression cassette is inserted between a pair of AAV inverted terminal repeats (ITRs). In some aspects, the AAV capsid proteins capsid proteins having 70% or more identity to AAV1, AAV2, AAV3, AAV4, AAV5, AAV6, AAV7, AAV8, AAV9, AAV10, AAV11, AAV12, AAV-rh74, AAV-Rh10, or AAV-2i8 VP1, VP2 and/or VP3 capsid proteins. In some aspects, the pair of AAV ITRs are ITRs having 70% or more identity to AAV1, AAV2, AAV3, AAV4, AAV5, AAV6, AAV7, AAV8, AAV9, AAV10, AAV11, AAV12, AAV-rh74, AAV-Rh10, or AAV-2i8 ITR sequence.

In one embodiment, provided herein are methods of treating a disease in a subject in need thereof. In one embodiment, the methods described herein comprise administering to the subject a nucleic acid composition of any one of the present embodiments (e.g., a therapeutically effective amount of the nucleic acid composition). In some aspects, the disease is caused by a protein deficiency. In some aspects, the nucleic acid composition encodes a therapeutic protein that is deficient in the disease. In some aspects, the subject has a cancer, an autoimmune disease, or a metabolic disorder.

In some aspects, the first and/or second and/or third expression cassette is comprised in a viral vector. In some aspects, the viral vector is selected from an adeno-associated viral (AAV) vector, a lentiviral vector, or a retroviral vector. In some aspects, the AAV vector comprises an AAV particle comprising AAV capsid proteins, and wherein the first and/or second and/or third expression cassette is inserted between a pair of AAV inverted terminal repeats (ITRs). In some aspects, the AAV capsid proteins are capsid proteins having 70% or more identity to AAV1, AAV2, AAV3, AAV4, AAV5, AAV6, AAV7, AAV8, AAV9, AAV10, AAV11, AAV12, AAV-rh74, AAV-Rh10, or AAV-2i8 VP1, VP2 and/or VP3 capsid proteins. In some aspects, the pair of AAV ITRs are ITRs having 70% or more identity to AAV1, AAV2, AAV3, AAV4, AAV5, AAV6, AAV7, AAV8, AAV9, AAV10, AAV11, AAV12, AAV-rh74, AAV-Rh10, or AAV-2i8 ITR sequence. In some aspects, a plurality of viral vectors is administered.

In one embodiment, provided herein are methods of treating a disease in a subject in need thereof. In one embodiment, the methods described herein comprise administering to the subject the cells of any one of the present embodiments (e.g., a therapeutically effective amount of the cells). In some aspects, the disease is caused by a protein deficiency. In some aspects, the cells express a therapeutic protein that is deficient in the disease. In some aspects, the subject has a cancer, an autoimmune disease, or a metabolic disorder.

In one embodiment, provided herein are nucleic acid compositions comprising (a) a first expression cassette comprising a minimal pol II promoter operably linked to a coding sequence for a therapeutic protein; (b) a second expression cassette comprising an inducible pol II promoter operably linked to a coding sequence of a dCas9-activator protein; and (c) a third expression cassette comprising a pol III promoter operably linked to a coding sequence for a guide RNA (gRNA), wherein the gRNA is able to hybridize to a sequence adjacent to the minimal pol II promoter that is present in the first expression cassette.

In some aspects, each expression cassette is located on a separate nucleic acid molecule. In some aspects, the nucleic acid molecules on which the first, the second, and the third expression cassettes are located each comprise an antibiotic resistance gene, wherein the antibiotic resistance genes on the first, the second, and the third expression cassettes are different. In some aspects, the antibiotic resistance genes each, independently, encode a protein that confers resistance to an antibiotic selected from neomycin, puromycin, blasticidin, zeocin, and hygromycin. The sequence of the protein encoded by the antibiotic resistance gene may be as depicted in Table 1. In some aspects, the antibiotic resistance gene on one or more of the nucleic acid molecules is flanked by a loxP site.

In some aspects, the nucleic acid molecule on which the third expression cassette is located further comprises a pol II promoter operably linked to a coding sequence for rTetR. In some aspects, the pol II promoter is an EF-1α promoter, a CMV promoter, a Ubc promoter, a PGK promoter, a VMD2 promoter, or a CAG promoter. The sequence of the promoter may be as depicted in Table 1. In some aspects, the pol II promoter is operably linked to a coding sequence of an antibiotic resistance gene and rTetR, wherein the sequence encoding the antibiotic resistance gene and the sequence encoding the rTetR are separated by a sequence encoding a cleavable peptide.

In some aspects, all three expression cassettes are located on a single nucleic acid molecule. In some aspects, the nucleic acid molecule further comprises an antibiotic resistance gene. In some aspects, the antibiotic resistance gene encodes a protein that confers resistance to an antibiotic selected from neomycin, puromycin, blasticidin, zeocin, and hygromycin. In some aspects, the antibiotic resistance gene is flanked by a loxP site.

In some aspects, the minimal pol II promoter is a cell-type specific promoter. In some aspects, the minimal pol II promoter is a minimal EF-1α promoter, a minimal CMV promoter, a minimal Ubc promoter, a minimal PGK promoter, a minimal VMD2 promoter, or a minimal CAG promoter. In some aspects, the inducible pol II promoter is a TRE promoter. In some aspects, the pol III promoter is a U6 promoter.

In some aspects, the first and/or second and/or third expression cassette comprises an enhancer element. In some aspects, the first and/or second expression cassette comprises an intron, a poly A signal, or a combination thereof. In some aspects, the first and/or second and/or third expression cassette comprises a filler polynucleotide.

In some aspects, the therapeutic protein is human IL-2, VEGF alpha, TGF beta, IL-4, IL-6, IL-7, IL-10, IL-12a, IL-12b, IL-15, Factor VII, Factor VIII, VWF, GCG, Factor IX, proenkephalin, EGF, IGF-1, TGF-beta1, hemoglobin (alpha-globin), hemoglobin (beta-globin), or bFGF. The sequence of the therapeutic protein may be as depicted in Table 1.

In some aspects, the first and/or second and/or third expression cassette is comprised in a viral vector. In some aspects, the viral vector is selected from an adeno-associated viral (AAV) vector, a lentiviral vector, or a retroviral vector.

In one embodiment, provided herein are cells comprising the nucleic acid composition of any one of the present embodiments. In some aspects, the expression cassettes are integrated into the genome of the cell. In some aspects, the cells express the therapeutic protein in the presence of a stimulus that activates the inducible promoter of the second expression cassette. In some aspects, the inducible promoter is a Tre promoter, wherein the cell expresses the therapeutic protein in the presence of doxycycline.

In one embodiment, provided herein are pharmaceutical compositions comprising the cells of any one of the present embodiments and a pharmaceutically acceptable carrier.

In one embodiment, provided herein are bioreactors comprising the cells of any one of the present embodiments.

In one embodiment, provided herein are methods of producing a therapeutic protein, the methods comprising culturing the cells of any one of the present embodiments under conditions to allow for expression of the therapeutic protein and isolating the therapeutic protein from the cells.

In one embodiment, provided herein are recombinant adeno-associated virus (rAAV) vectors comprising an AAV capsid protein and the nucleic acid composition of any one of the present embodiments. In some aspects, the AAV vector comprises an AAV particle comprising AAV capsid proteins, and wherein the first and/or second and/or third expression cassette is inserted between a pair of AAV inverted terminal repeats (ITRs). In some aspects, the AAV capsid proteins are capsid proteins having 70% or more identity to AAV1, AAV2, AAV3, AAV4, AAV5, AAV6, AAV7, AAV8, AAV9, AAV10, AAV11, AAV12, AAV-rh74, AAV-Rh10, or AAV-2i8 VP1, VP2 and/or VP3 capsid proteins. In some aspects, the pair of AAV ITRs are ITRs having 70% or more identity to AAV1, AAV2, AAV3, AAV4, AAV5, AAV6, AAV7, AAV8, AAV9, AAV10, AAV11, AAV12, AAV-rh74, AAV-Rh10, or AAV-2i8 ITR sequence.

In one embodiment, provided herein are methods of treating a disease in a subject in need thereof. In one embodiment, the methods described herein comprise administering to the subject a nucleic acid composition of any one of the present embodiments (e.g., a therapeutically effective amount of the nucleic acid composition). In some aspects, the disease is caused by a protein deficiency. In some aspects, the nucleic acid composition encodes a therapeutic protein that is deficient in the disease. In some aspects, the subject has a cancer, an autoimmune disease, or a metabolic disorder.

In some aspects, the first and/or second and/or third expression cassette is comprised in a viral vector. In some aspects, the viral vector is selected from an adeno-associated viral (AAV) vector, a lentiviral vector, or a retroviral vector. In some aspects, the AAV vector comprises an AAV particle comprising AAV capsid proteins, and wherein the first and/or second and/or third expression cassette is inserted between a pair of AAV inverted terminal repeats (ITRs). In some aspects, the AAV capsid proteins are capsid proteins having 70% or more identity to AAV1, AAV2, AAV3, AAV4, AAV5, AAV6, AAV7, AAV8, AAV9, AAV10, AAV11, AAV12, AAV-rh74, AAV-Rh10, or AAV-2i8 VP1, VP2 and/or VP3 capsid proteins. In some aspects, the pair of AAV ITRs are ITRs having 70% or more identity to AAV1, AAV2, AAV3, AAV4, AAV5, AAV6, AAV7, AAV8, AAV9, AAV10, AAV11, AAV12, AAV-rh74, AAV-Rh10, or AAV-2i8 ITR sequence. In some aspects, a plurality of viral vectors is administered.

In one embodiment, provided herein are methods of treating a disease in a subject in need thereof. In one embodiment, the methods described herein comprise administering to the subject the cells of any one of the present embodiments (e.g., a therapeutically effective amount of the cells). In some aspects, the disease is caused by a protein deficiency. In some aspects, the cells express a therapeutic protein that is deficient in the disease. In some aspects, the subject has a cancer, an autoimmune disease, or a metabolic disorder.

Also provided herein are:

1. A nucleic acid composition comprising (a) a first expression cassette comprising a pol II promoter operably linked to a coding sequence for a therapeutic protein, wherein the pol II promoter is a constitutive promoter; (b) a second expression cassette comprising a pol II promoter operably linked to a coding sequence of a dCas9-activator protein; and (c) a third expression cassette comprising a pol III promoter operably linked to a coding sequence for a guide RNA (gRNA), wherein the gRNA is able to hybridize to the pol II promoter that is present in both the first and second expression cassettes.
2. The nucleic acid composition of aspect 1, wherein the pol II promoter in a) and in b) are the same promoter.
3. The nucleic acid composition of aspect 1, wherein the pol II promoter in a) and in b) are different, wherein the gRNA is able to hybridize to the pol II promoters in a) and in b).
4. The nucleic acid composition of aspect 1, wherein each expression cassette is located on a separate nucleic acid molecule.
5. The nucleic acid composition of aspect 4, wherein the nucleic acid molecules on which the first, the second, and the third expression cassettes are located each comprise an antibiotic resistance gene, wherein the antibiotic resistance genes on the first, the second, and the third expression cassettes are different.
6. The nucleic acid composition of aspect 5, wherein the antibiotic resistance genes each, independently, encode a protein that confers resistance to an antibiotic selected from neomycin, puromycin, blasticidin, zeocin, and hygromycin.
7. The nucleic acid composition of aspect 5, wherein the antibiotic resistance gene on one or more of the nucleic acid molecules is flanked by a loxP site.
8. The nucleic acid composition of aspect 1, wherein all three expression cassettes are located on a single nucleic acid molecule.
9. The nucleic acid composition of aspect 8, wherein the nucleic acid molecule further comprises an antibiotic resistance gene.
10. The nucleic acid composition of aspect 9, wherein the antibiotic resistance gene encodes a protein that confers resistance to an antibiotic selected from neomycin, puromycin, blasticidin, zeocin, and hygromycin.
11. The nucleic acid composition of aspect 9 or 10, wherein the antibiotic resistance gene is flanked by a loxP site.
12. The nucleic acid composition of any one of aspects 1-11, wherein the pol II promoter is a cell-type specific promoter.
13. The nucleic acid composition of any one of aspects 1-11, wherein the pol II promoter is an EF-1α promoter, a CMV promoter, a Ubc promoter, a PGK promoter, a VMD2 promoter, or a CAG promoter.
14. The nucleic acid composition of any one of aspects 1-13, wherein the pol III promoter is a U6 promoter.
15. The nucleic acid composition of any one of aspects 1-14, wherein the first and/or second and/or third expression cassette comprises an enhancer element.
16. The nucleic acid composition of any one of aspects 1-15, wherein the first and/or second expression cassette comprises an intron, a poly A signal, or a combination thereof.
17. The nucleic acid composition of any one of aspects 1-16, wherein the first and/or second and/or third expression cassette comprises a filler polynucleotide.
18. The nucleic acid composition of any one of aspects 1-17, wherein the therapeutic protein is human IL-2, VEGF alpha, TGF beta, IL-4, IL-6, IL-7, IL-10, IL-12a, IL-12b, IL-15, Factor VII, Factor VIII, VWF, GCG, Factor IX, proenkephalin, EGF, IGF-1, TGF-beta1, hemoglobin (alpha-globin), hemoglobin (beta-globin), or bFGF.
19. The nucleic acid composition of any one of aspects 1-18, wherein the first and/or second and/or third expression cassette is comprised in a viral vector.
20. The nucleic acid composition of aspect 19, wherein the viral vector is selected from an adeno-associated viral (AAV) vector, a lentiviral vector, or a retroviral vector.
21. A cell comprising the nucleic acid composition of any one of aspects 1-20.
22. The cell of aspect 21, wherein the expression cassettes are integrated into the genome of the cell.
23. The cell of aspect 21 or 22, wherein the cell expresses the therapeutic protein.
24. The cell of aspect 23, wherein the expression level of the therapeutic protein is stable over time.
25. The cell of any one of aspects 21-24, wherein the pol II promoter drives a high level of transcription in that cell.
26. A pharmaceutical composition comprising the cell of any one of aspects 21-24 and a pharmaceutically acceptable carrier.
27. A bioreactor comprising the cell of any one of aspects 21-24.
28. A method of producing a therapeutic protein, the method comprising culturing the cells of any one of aspects 21-25 under conditions to allow for expression of the therapeutic protein and isolating the therapeutic protein from the cells.
29. A recombinant adeno-associated virus (rAAV) vector comprising an AAV capsid protein and the nucleic acid composition of any one of aspects 8-20.
30. The rAAV of aspect 29, wherein the AAV vector comprises an AAV particle comprising AAV capsid proteins, and wherein the first and/or second and/or third expression cassette is inserted between a pair of AAV inverted terminal repeats (ITRs).
31. The rAAV of aspect 30, wherein the AAV capsid proteins capsid proteins having 70% or more identity to AAV1, AAV2, AAV3, AAV4, AAV5, AAV6, AAV7, AAV8, AAV9, AAV10, AAV11, AAV12, AAV-rh74, AAV-Rh10, or AAV-2i8 VP1, VP2 and/or VP3 capsid proteins.
32. The rAAV of aspect 30, wherein the pair of AAV ITRs are ITRs having 70% or more identity to AAV1, AAV2, AAV3, AAV4, AAV5, AAV6, AAV7, AAV8, AAV9, AAV10, AAV11, AAV12, AAV-rh74, AAV-Rh10, or AAV-2i8 ITR sequence.
33. A method of treating a disease in a subject in need thereof, the method comprising administering to the subject a therapeutically effective amount of a nucleic acid composition of any one of aspects 1-18.
34. The method of aspect 33, wherein the disease is caused by a protein deficiency.
35. The method of aspect 33 or 34, wherein the nucleic acid composition encodes a therapeutic protein that is deficient in the disease.
36. The method of aspect 33, wherein the subject has a cancer, an autoimmune disease, or a metabolic disorder.
37. The method of any one of aspects 33-36, wherein the first and/or second and/or third expression cassette is comprised in a viral vector.
38. The method of aspect 37, wherein the viral vector is selected from an adeno-associated viral (AAV) vector, a lentiviral vector, or a retroviral vector.
39. The method of aspect 38, wherein the AAV vector comprises an AAV particle comprising AAV capsid proteins, and wherein the first and/or second and/or third expression cassette is inserted between a pair of AAV inverted terminal repeats (ITRs).
40. The method of aspect 39, wherein the AAV capsid proteins are capsid proteins having 70% or more identity to AAV1, AAV2, AAV3, AAV4, AAV5, AAV6, AAV7, AAV8, AAV9, AAV10, AAV11, AAV12, AAV-rh74, AAV-Rh10, or AAV-2i8 VP1, VP2 and/or VP3 capsid proteins.
41. The method of aspect 39, wherein the pair of AAV ITRs are ITRs having 70% or more identity to AAV1, AAV2, AAV3, AAV4, AAV5, AAV6, AAV7, AAV8, AAV9, AAV10, AAV11, AAV12, AAV-rh74, AAV-Rh10, or AAV-2i8 ITR sequence.
42. The method of any one of aspects 37-41, wherein a plurality of viral vectors is administered.
43. A method of treating a disease in a subject in need thereof, the method comprising administering to the subject a therapeutically effective amount of the cells of any one of aspects 21-24.
44. The method of aspect 43, wherein the disease is caused by a protein deficiency.
45. The method of aspect 43 or 44, wherein the cells express a therapeutic protein that is deficient in the disease.
46. The method of aspect 43, wherein the subject has a cancer, an autoimmune disease, or a metabolic disorder.
47. A nucleic acid composition comprising (a) a first expression cassette comprising a minimal pol II promoter operably linked to a coding sequence for a therapeutic protein; (b) a second expression cassette comprising an inducible pol II promoter operably linked to a coding sequence of a dCas9-activator protein; and (c) a third expression cassette comprising a pol III promoter operably linked to a coding sequence for a guide RNA (gRNA), wherein the gRNA is able to hybridize to a sequence adjacent to the minimal pol II promoter that is present in the first expression cassette.
48. The nucleic acid composition of aspect 47, wherein each expression cassette is located on a separate nucleic acid molecule.
49. The nucleic acid composition of aspect 48, wherein the nucleic acid molecules on which the first, the second, and the third expression cassettes are located each comprise an antibiotic resistance gene, wherein the antibiotic resistance genes on the first, the second, and the third expression cassettes are different.
50. The nucleic acid composition of aspect 49, wherein the antibiotic resistance genes each, independently, encode a protein that confers resistance to an antibiotic selected from neomycin, puromycin, blasticidin, zeocin, and hygromycin.
51. The nucleic acid composition of aspect 49, wherein the antibiotic resistance gene on one or more of the nucleic acid molecules is flanked by a loxP site.
52. The nucleic acid composition of aspect 48, wherein the nucleic acid molecule on which the third expression cassette is located further comprises a pol II promoter operably linked to a coding sequence for rTetR.
53. The nucleic acid composition of aspect 52, wherein the pol II promoter is an EF-1α promoter, a CMV promoter, a Ubc promoter, a PGK promoter, a VMD2 promoter, or a CAG promoter.
54. The nucleic acid composition of aspect 52, wherein the pol II promoter is operably linked to a coding sequence of an antibiotic resistance gene and rTetR, wherein the sequence encoding the antibiotic resistance gene and the sequence encoding the rTetR are separated by a sequence encoding a cleavable peptide.
55. The nucleic acid composition of aspect 47, wherein all three expression cassettes are located on a single nucleic acid molecule.
56. The nucleic acid composition of aspect 55, wherein the nucleic acid molecule further comprises an antibiotic resistance gene.
57. The nucleic acid composition of aspect 56, wherein the antibiotic resistance gene encodes a protein that confers resistance to an antibiotic selected from neomycin, puromycin, blasticidin, zeocin, and hygromycin.
58. The nucleic acid composition of aspect 55 or 56, wherein the antibiotic resistance gene is flanked by a loxP site.
59. The nucleic acid composition of any one of aspects 47-58, wherein the minimal pol II promoter is a cell-type specific promoter.
60. The nucleic acid composition of any one of aspects 47-58, wherein the minimal pol II promoter is a minimal EF-1α promoter, a minimal CMV promoter, a minimal Ubc promoter, a minimal PGK promoter, a minimal VMD2 promoter, or a minimal CAG promoter.
61. The nucleic acid composition of any one of aspects 47-60, wherein the inducible pol II promoter is a TRE promoter.
62. The nucleic acid composition of any one of aspects 47-61, wherein the pol III promoter is a U6 promoter.
63. The nucleic acid composition of any one of aspects 47-62, wherein the first and/or second and/or third expression cassette comprises an enhancer element.
64. The nucleic acid composition of any one of aspects 47-63, wherein the first and/or second expression cassette comprises an intron, a poly A signal, or a combination thereof.
65. The nucleic acid composition of any one of aspects 47-64, wherein the first and/or second and/or third expression cassette comprises a filler polynucleotide.
66. The nucleic acid composition of any one of aspects 47-65, wherein the therapeutic protein is human IL-2, VEGF alpha, TGF beta, IL-4, IL-6, IL-7, IL-10, IL-12a, IL-12b, IL-15, Factor VII, Factor VIII, VWF, GCG, Factor IX, proenkephalin, EGF, IGF-1, TGF-beta1, hemoglobin (alpha-globin), hemoglobin (beta-globin), or bFGF.
67. The nucleic acid composition of any one of aspects 47-66, wherein the first and/or second and/or third expression cassette is comprised in a viral vector.
68. The nucleic acid composition of aspect 67, wherein the viral vector is selected from an adeno-associated viral (AAV) vector, a lentiviral vector, or a retroviral vector.
69. A cell comprising the nucleic acid composition of any one of aspects 47-68.
70. The cell of aspect 69, wherein the expression cassettes are integrated into the genome of the cell.
71. The cell of aspect 69 or 70, wherein the cell expresses the therapeutic protein in the presence of a stimulus that activates the inducible promoter of the second expression cassette.
72. The cell of aspect 71, wherein the inducible promoter is a Tre promoter, wherein the cell expresses the therapeutic protein in the presence of doxycycline.
73. A pharmaceutical composition comprising the cell of any one of aspects 69-72 and a pharmaceutically acceptable carrier.
74. A bioreactor comprising the cell of any one of aspects 69-72.
75. A method of producing a therapeutic protein, the method comprising culturing the cells of any one of aspects 69-72 under conditions to allow for expression of the therapeutic protein and isolating the therapeutic protein from the cells.
76. A recombinant adeno-associated virus (rAAV) vector comprising an AAV capsid protein and the nucleic acid composition of any one of aspects 55-68.
77. The rAAV of aspect 76, wherein the AAV vector comprises an AAV particle comprising AAV capsid proteins, and wherein the first and/or second and/or third expression cassette is inserted between a pair of AAV inverted terminal repeats (ITRs).
78. The rAAV of aspect 77, wherein the AAV capsid proteins are capsid proteins having 70% or more identity to AAV1, AAV2, AAV3, AAV4, AAV5, AAV6, AAV7, AAV8, AAV9, AAV10, AAV11, AAV12, AAV-rh74, AAV-Rh10, or AAV-2i8 VP1, VP2 and/or VP3 capsid proteins.
79. The rAAV of aspect 77, wherein the pair of AAV ITRs are ITRs having 70% or more identity to AAV1, AAV2, AAV3, AAV4, AAV5, AAV6, AAV7, AAV8, AAV9, AAV10, AAV11, AAV12, AAV-rh74, AAV-Rh10, or AAV-2i8 ITR sequence.
80. A method of treating a disease in a subject in need thereof, the method comprising administering to the subject a therapeutically effective amount of a nucleic acid composition of any one of aspects 47-68.
81. The method of aspect 80, wherein the disease is caused by a protein deficiency.
82. The method of aspect 80 or 81, wherein the nucleic acid composition encodes a therapeutic protein that is deficient in the disease.
83. The method of aspect 80, wherein the subject has a cancer, an autoimmune disease, or a metabolic disorder.
84. The method of any one of aspects 80-83, wherein the first and/or second and/or third expression cassette is comprised in a viral vector.
85. The method of aspect 84, wherein the viral vector is selected from an adeno-associated viral (AAV) vector, a lentiviral vector, or a retroviral vector.
86. The method of aspect 85, wherein the AAV vector comprises an AAV particle comprising AAV capsid proteins, and wherein the first and/or second and/or third expression cassette is inserted between a pair of AAV inverted terminal repeats (ITRs).
87. The method of aspect 86, wherein the AAV capsid proteins are capsid proteins having 70% or more identity to AAV1, AAV2, AAV3, AAV4, AAV5, AAV6, AAV7, AAV8, AAV9, AAV10, AAV11, AAV12, AAV-rh74, AAV-Rh10, or AAV-2i8 VP1, VP2 and/or VP3 capsid proteins.
88. The method of aspect 86, wherein the pair of AAV ITRs are ITRs having 70% or more identity to AAV1, AAV2, AAV3, AAV4, AAV5, AAV6, AAV7, AAV8, AAV9, AAV10, AAV11, AAV12, AAV-rh74, AAV-Rh10, or AAV-2i8 ITR sequence.
89. The method of any one of aspects 84-88, wherein a plurality of viral vectors is administered.
90. A method of treating a disease in a subject in need thereof, the method comprising administering to the subject a therapeutically effective amount of the cells of any one of aspects 69-72.
91. The method of aspect 90, wherein the disease is caused by a protein deficiency.
92. The method of aspect 90 or 91, wherein the cells express a therapeutic protein that is deficient in the disease.
93. The method of aspect 90, wherein the subject has a cancer, an autoimmune disease, or a metabolic disorder.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, the variation that exists among the study subjects, or a value that is within 10% (plus or minus) of a reference value.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. The vector construction of eGFP gene under different promoters.

FIG. 2. The transient expression of eGFP under different promoters in HEK293T cells. The eGFP intensity was measured by fluorescent microscopy and FACS.

FIG. 3. The expression of eGFP under different promoters in HEK293T cells following lentiviral transduction. The eGFP intensity was measured by fluorescent microscopy and FACS.

FIG. 4. The expression of eGFP under different promoters in ARPE-19 cells following lentiviral transduction at MOI=0.1.

FIG. 5. The expression of eGFP in ARPE-19 cells following lentiviral transduction. The eGFP intensity was measured by both fluorescent microscopy and FACS.

FIG. 6. eGFP expression under CAG promoter in ARPE19 cells following lentiviral transduction at different MOI.

FIG. 7. eGFP expression under CMV promoter in ARPE19 cells following lentiviral transduction at different MOI.

FIG. 8. Schematic of the gRNA locus at CAG promoter.

FIG. 9. Schematic of constitutive gene expression system in RPE cells.

FIG. 10. Schematic of random integration of constitutive gene expression system in RPE cells.

FIG. 11. Schematic of constitutive expression system in AAVSI.

FIG. 12. Schematic of inducible gene expression system in RPE cells.

FIG. 13. Schematic of random integration of inducible gene expression system in RPE cells.

FIG. 14. Schematic of inducible gene expression system at AAVSI.

### DETAILED DESCRIPTION

Provided herein are cassettes that enable sustained and/or inducible, tightly controlled production of genetically encoded payloads from engineered cells. These cassettes may allow cells to be engineered to express genetically encoded payloads despite epigenetic silencing.

For constitutive expression, the system comprises three expression cassettes, with each cassette encoding one of a dCas9-Activator protein, a guide RNA, and a therapeutic product. The therapeutic product may be a therapeutic protein or a therapeutic nucleic acid (e.g., RNA). The therapeutic product coding sequence and the dCas9-Activator protein coding sequence are operably linked to a constitutive promoter, such as, for example, the EF-1α, CMV, Ubc, hPGK, VMD2, or CAG promoter. The constitutive promoter may have been determined to be a strong promoter in the target cell for the system. The guide RNA coding sequence is operably linked to a constitutive pol III promoter, such as, for example, the U6 promoter. The guide RNA sequence targets, e.g., is designed to target, both the constitutive promoter that is operably linked to the therapeutic protein coding sequence and the dCas9-Activator protein coding sequence, thereby targeting the expressed dCas9-Activator protein to the constitutive promoter and, e.g., facilitating expression of the therapeutic protein and the dCas9-Activator protein. In an embodiment, the two promoters are identical. In an embodiment, the two promoters are different, but each has a sequence which interacts with the guide RNA. This creates a positive feedback loop within the system.

For inducible expression, the system also comprises three expression cassettes, with each cassette encoding one of a dCas9-Activator protein, a guide RNA, and a therapeutic product. However, the therapeutic product coding sequence is operably linked to a minimal promoter, such as, for example, a minimal CMV promoter (Li et al., Gene Ther., 16:43-51, 2009). The minimal promoter can be any promoter that drives very low basal expression. The dCas9-Activator protein coding sequence is operably linked to an inducible promoter, such as, for example, a doxycycline-inducible Tre promoter (c.f. Gossen et al., “Tight control of gene expression in mammalian by tetracycline-responsive promoters,” Proc. Natl. Acad. Sci. U.S.A., 89:5547-5551, 1992). The guide RNA coding sequence is operably linked to a constitutive pol III promoter, such as, for example, the U6 promoter. The guide RNA sequence targets, e.g., is designed to target, the minimal promoter that is operably linked to the therapeutic protein coding sequence, thereby targeting the expressed dCas9-Activator protein to the minimal promoter and inducing expression of the therapeutic protein in the presence of the inducing factor for the inducible promoter. In an embodiment, the two promoters are identical. In an embodiment, the two promoters are different, but each has a sequence which interacts with the guide RNA.

In either system, the three expression cassettes may be present on separate nucleic acid molecules. In that case, each of the nucleic acid molecules has its own antibiotic resistance gene (e.g., a gene that expresses a protein that confers resistance to an antibiotic selected from neomycin, puromycin, blasticidin, zeocin, and hygromycin) to allow for selection of cells having the nucleic acid molecules. In order to reduce the toxicity of antibiotic resistance, the antibiotic resistance gene cassettes may be flanked by a LoxP site allowing for the antibiotic resistance gene cassettes to be removed by introducing Cre enzymes into the cells. Alternatively, the three expression cassettes may be present on a single nucleic acid molecule. In that case, the single nucleic acid molecule has an antibiotic resistance gene to allow for selection of cells having the nucleic acid molecule.

The cells that can be transduced with a system of expression cassettes as provided herein include any target cell type of interest. The target cell may be a murine mesenchymal stem cell (e.g., EK8), a human mesenchymal stem cell, a HUVEC cell, a 293HEK cell, a Jurkat T cell, a HeLa cell, an NIH3T3 cell, a CHO-K1 cell, a COS-1 cell, a COS-7 cell, a PC-3 cell, an HCT 116 cell, an A549MCF-7 cell, a HuH-7 cell, a U-2 OS cell, a HepG2 cell, a Neuro-2a cell, a primary MEF cells, a human primary fibroblast cell, or an SF9 cell. The target cell may be a cell inside a subject. The target cell may be an ex vivo cell.

### I. CRISPR dCas9-Activator Systems

Gene editing is a technology that allows for the modification of target genes within living cells. Recently, harnessing the bacterial immune system of CRISPR to perform on-demand gene editing revolutionized the way scientists approach genomic editing. The Cas9 protein of the CRISPR system, which is an RNA guided DNA endonuclease, can be engineered to target new sites with relative ease by altering its guide RNA sequence. This discovery has made sequence specific gene editing functionally effective.

In addition, the sequence specificity of CRISPR systems has been taken advantage of in order to develop systems for modulating gene expression, rather than editing a genomic sequence. In this context, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

The CRISPR/Cas activator system can include a non-coding guide RNA molecule (gRNA), which sequence-specifically binds to DNA, and a catalytically inactive Cas protein (e.g., Cas9) fused to a heterologous effector domain, such as a transcriptional activator. A CRISPR system with a catalytically inactivate Cas9 may further comprise a transcriptional activator fused to a ribosomal binding protein. One or more elements of a CRISPR system can derive from a type I, type II, or type III CRISPR system, e.g., derived from a particular organism comprising an endogenous CRISPR system, such as Streptococcus pyogenes, Staphylococcus aureus, or Lachnospiraceae bacterium.

In some aspects, a Cas nuclease and gRNA (including a fusion of crRNA specific for the target sequence and fixed tracrRNA) are each independently introduced into a cell. In general, target sites at the 5′ end of the gRNA target the Cas nuclease to the target site, e.g., the promoter, using complementary base pairing. The gRNA is targeted to the desired sequence by modifying the first 20, 19, 18, 17, 16, 15, 14, 13, 12, 11, or 10 nucleotides of the guide RNA to correspond to the target DNA sequence. In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

Typically, in the context of an endogenous CRISPR system, formation of the CRISPR complex (comprising the guide sequence hybridized to the target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. However, in the case of a catalytically inactive Cas9 CRISPR/Cas activator system, formation of the CRISPR complex results in, for example, transcriptional activation of a nearby promoter when the heterologous effector domain is a transcriptional activator.

One or more vectors driving expression of one or more elements of the CRISPR system can be introduced into a cell such that expression of the elements of the CRISPR system direct formation of the CRISPR complex at one or more target sites. For example, a Cas9 protein and a gRNA could each be operably linked to separate regulatory elements on separate vectors. The gRNA may be under the control of a constitutive promoter, such as, for example, a U6 promoter (Cong et al., “Multiplex genome engineering using CRISPR/Cas systems,” Science, 339:819-823, 2013; Mali et al., “RNA-guided human genome engineering via Cas9,” Science, 339:823-826, 2013). The Cas9 protein may be under the control of a constitutive, strong promoter in the event that the genetically-encoded payload is sought to be constitutively express. Alternatively, the Cas9 protein may be under the control of an inducible promoter in the event that the genetically-encoded payload is sought to be inducibly expressed.

Alternatively, two or more of the elements expressed from the same or different regulatory elements, may be combined in a single vector, with one or more additional vectors providing any components of the CRISPR system not included in the first vector. The vector may comprise one or more insertion sites, such as a restriction endonuclease recognition sequence (also referred to as a “cloning site”). In some embodiments, one or more insertion sites are located upstream and/or downstream of one or more sequence elements of one or more vectors. When multiple different guide sequences are used, a single expression construct may be used to target CRISPR activity to multiple different, corresponding target sequences within a cell.

A vector may comprise a regulatory element operably linked to a protein-coding sequence encoding the CRISPR enzyme, such as a Cas protein. Non-limiting examples of Cas proteins include Cas1, Cas1B, Cas2, Cas3, Cas4, Cas5, Cash, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2. In some instances, the Cas9 protein may be Streptococcus pyogenes Cas9, Staphylococcus aureus Cas9, or Lachnospiraceae bacterium Cpf1.

The CRISPR enzyme can be catalytically inactive Cas9 (e.g., from S. pyogenes or S. pneumonia). In other words, the vector can encode a CRISPR enzyme that is mutated with respect to a corresponding wild-type enzyme such that the mutated CRISPR enzyme lacks the ability to cleave a target polynucleotide containing a target sequence. The CRISPR enzyme may be part of a fusion protein comprising one or more heterologous protein domains (see, e.g., U.S. Patent Application Publication No. 2018/0291370; Chavez et al., Nat. Methods, 12:326-328, 2015). A CRISPR enzyme fusion protein may comprise any additional protein sequence, and optionally a linker sequence between any two domains. Examples of protein domains that may be fused to a CRISPR enzyme include, without limitation, protein domains having one or more of the following activities: methylase activity (McDonald et al., “Reprogrammable CRISPR/Cas9-based system for inducing site-specific DNA methylation,” Biology Open, 5:866-874, 2016), demethylase activity (Amabile et al., “Inheritable silencing of endogenous genes by hit-and-run targeted epigenetic editing,” Cell, 167:219-232, 2016), transcription activation activity (Hu et al., “Direct activation of human and mouse Oct4 genes using engineered TALE and Cas9 transcription factors,” Nuc. Acids Res., 42:4375-4390, 2014; Hilton et al, “CRISPR-Cas9-based acetyltransferase activates genes from promoters and enhancers,” Nat. Biotechnol., 33:510-517, 2015; U.S. Patent Application Publication No. 2018/0023064), transcription repression activity (Thakore et al., “Highly specific epigenome editing by CRISPR-Cas9 repressors for silencing of distal regulatory elements,” Nat. Meth., 12:1143-1149, 2015), transcription release factor activity, histone modification activity, RNA cleavage activity, and nucleic acid binding activity. A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions. In certain instances, the CRISPR enzyme fusion protein may comprise the TET1 catalytic domain, the P300 core, VPR, or rTetR. Additional domains that may form part of a fusion protein comprising a CRISPR enzyme are described in U.S. Patent Appln. Publn. US 2011/0059502, incorporated herein by reference.

An enzyme coding sequence encoding the CRISPR enzyme may be codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

In general, a guide sequence is any polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), Clustal W, Clustal X, BLAT, Novoalign (Novocraft Technologies, ELAND (Illumina, San Diego, Calif.), SOAP (available at soap.genomics.org.cn), and Maq (available at maq.sourceforge.net).

### II. Therapeutic Proteins

Examples of therapeutic proteins that may be expressed using the expression systems of the present disclosure include, but are not limited to, VEGFα, TGFβ, IL2, IL4, IL6, IL7, IL10, IL12a, IL12b, IL15, factor VII, factor VIII, VWF, GCG, factor IX, proenkephalin, EGF, IGF-1, TGF,β1, hemoglobin (α-globin), hemoglobin (β-globin), or bFGF. In some cases, the therapeutic protein may be fused to a cell export peptide, such as, for example, a human OSM, VSV-G, mouse Ig kappa, human IgG2 H, BM40, secrecon, human IgKVIII, CD33, tPA, human chymotryposinogen, human trypsinogen-2, human IL-2, gaussian luc, albumin (HSA), influenza haemagglutinin, human insulin, or silkworm fibroin LC cell export peptide.

In some aspects, the protein or polypeptide may be modified to increase stability. Thus, when the present application refers to the function or activity of “modified protein” or a “modified polypeptide,” one of ordinary skill in the art would understand that this includes, for example, a protein or polypeptide that possesses an additional advantage over the unmodified protein or polypeptide. It is specifically contemplated that embodiments concerning a “modified protein” may be implemented with respect to a “modified polypeptide,” and vice versa.

Recombinant proteins may possess deletions and/or substitutions of amino acids; thus, a protein with a deletion, a protein with a substitution, and a protein with a deletion and a substitution are modified proteins. In some embodiments, these proteins may further include insertions or added amino acids, such as with fusion proteins or proteins with linkers, for example. A “modified deleted protein” lacks one or more residues of the native protein, but may possess the specificity and/or activity of the native protein. A “modified deleted protein” may also have reduced immunogenicity or antigenicity. An example of a modified deleted protein is one that has an amino acid residue deleted from at least one antigenic region that is, a region of the protein determined to be antigenic in a particular organism, such as the type of organism that may be administered the modified protein.

Substitution or replacement variants typically contain the exchange of one amino acid for another at one or more sites within the protein and may be designed to modulate one or more properties of the polypeptide, particularly its effector functions and/or bioavailability. Substitutions may or may not be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine, or methionine; serine to threonine; threonine to serine; tryptophan to tyrosine; tyrosine to tryptophan or phenylalanine; and valine to isoleucine or leucine. In addition to a deletion or substitution, a modified protein may possess an insertion of residues, which typically involves the addition of at least one residue in the polypeptide. This may include the insertion of a targeting peptide or polypeptide or simply a single residue.

The term “biologically functional equivalent” is well understood in the art and is further defined in detail herein. Accordingly, sequences that have between about 70% and about 80%, or between about 81% and about 90%, or even between about 91% and about 99% of amino acids that are identical or functionally equivalent to the amino acids of a control polypeptide are included, provided the biological activity of the protein is maintained. A recombinant protein may be biologically functionally equivalent to its native counterpart in certain aspects.

It also will be understood that amino acid and nucleic acid sequences may include additional residues, such as additional N- or C-terminal amino acids or 5′ or 3′ sequences, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region or may include various internal sequences, i.e., introns, which are known to occur within genes.

As used herein, a protein or peptide generally refers, but is not limited to, a protein of greater than about 200 amino acids, up to a full-length sequence translated from a gene; a polypeptide of greater than about 100 amino acids; and/or a peptide of from about 3 to about 100 amino acids. For convenience, the terms “protein,” “polypeptide,” and “peptide are used interchangeably herein.

### III. Methods of Delivering Nucleic Acid Cassettes to Cells

A. Viral Vectors

The term “vector” refers to carrier nucleic acid molecule, a plasmid, virus, or other vehicle that can be manipulated by insertion or incorporation of a nucleic acid. Vectors, such as viral vectors, can be used to introduce/transfer nucleic acid sequences into cells, such that the nucleic acid sequence therein is transcribed and, if encoding a protein, subsequently translated by the cells.

An “expression vector” is a specialized vector that contains a gene or nucleic acid sequence with the necessary regulatory regions needed for expression in a host cell. An expression vector may contain at least an origin of replication for propagation in a cell and optionally additional elements, such as a heterologous nucleic acid sequence, expression control element (e.g., a promoter), intron, ITR(s), and polyadenylation signal.

A viral vector is derived from or based upon one or more nucleic acid elements that comprise a viral genome. Exemplary viral vectors include adeno-associated virus (AAV) vectors, retroviral vectors, and lentiviral vectors.

The term “recombinant,” as a modifier of vector, such as recombinant viral, e.g., lenti- or parvo-virus (e.g., AAV) vectors, as well as a modifier of sequences such as recombinant nucleic acid sequences and polypeptides, means that the compositions have been manipulated (i.e., engineered) in a fashion that generally does not occur in nature. A particular example of a recombinant vector, such as an AAV, retroviral, or lentiviral vector would be where a nucleic acid sequence that is not normally present in the wild-type viral genome is inserted within the viral genome. An example of a recombinant nucleic acid sequence would be where a nucleic acid (e.g., gene) encodes a therapeutic protein that is cloned into a vector, with or without 5′, 3′ and/or intron regions that the gene is normally associated within the genome. Although the term “recombinant” is not always used herein in reference to vectors, such as viral vectors, as well as sequences such as polynucleotides, “recombinant” forms including nucleic acid sequences, polynucleotides, transgenes, etc. are expressly included in spite of any such omission.

A recombinant viral “vector” is derived from the wild type genome of a virus, such as AAV, retrovirus, or lentivirus, by using molecular methods to remove the wild type genome from the virus, and replacing with a non-native nucleic acid, such as a nucleic acid sequence. Typically, for example, for AAV, one or both inverted terminal repeat (ITR) sequences of the AAV genome are retained in the recombinant AAV vector. A “recombinant” viral vector (e.g., rAAV) is distinguished from a viral (e.g., AAV) genome, since all or a part of the viral genome has been replaced with a non-native sequence, such as a nucleic acid encoding a therapeutic protein and/or nucleic acid encoding an Cas9 protein and/or nucleic acid encoding a gRNA. Incorporation of such non-native nucleic acid sequences therefore defines the viral vector as a “recombinant” vector, which in the case of AAV can be referred to as a “rAAV vector.”

2. Adeno-Associated Virus

Adeno-associated virus (AAV) is a small nonpathogenic virus of the parvoviridae family. To date, numerous serologically distinct AAVs have been identified, and more than a dozen have been isolated from humans or primates. AAV is distinct from other members of this family by its dependence upon a helper virus for replication.

AAV genomes can exist in an extrachromosomal state without integrating into host cellular genomes; possess a broad host range; transduce both dividing and non-dividing cells in vitro and in vivo and maintain high levels of expression of the transduced genes. AAV viral particles are heat stable; resistant to solvents, detergents, changes in pH, and temperature; and can be column purified and/or concentrated on CsCl gradients or by other means. The AAV genome comprises a single-stranded deoxyribonucleic acid (ssDNA), either positive- or negative-sensed. The approximately 5 kb genome of AAV consists of one segment of single stranded DNA of either plus or minus polarity. The ends of the genome are short inverted terminal repeats (ITRs) that can fold into hairpin structures and serve as the origin of viral DNA replication.

An AAV “genome” refers to a recombinant nucleic acid sequence that is ultimately packaged or encapsulated to form an AAV particle. An AAV particle often comprises an AAV genome packaged with AAV capsid proteins. In cases where recombinant plasmids are used to construct or manufacture recombinant vectors, the AAV vector genome does not include the portion of the “plasmid” that does not correspond to the vector genome sequence of the recombinant plasmid. This non vector genome portion of the recombinant plasmid is referred to as the “plasmid backbone,” which is important for cloning and amplification of the plasmid, a process that is needed for propagation and recombinant virus production, but is not itself packaged or encapsulated into viral particles. Thus, an AAV vector “genome” refers to nucleic acid that is packaged or encapsulated by AAV capsid proteins.

The AAV virion (particle) is a non-enveloped, icosahedral particle approximately 25 nm in diameter. The AAV particle comprises an icosahedral symmetry comprised of three related capsid proteins, VP1, VP2 and VP3, which interact together to form the capsid. The right ORF often encodes the capsid proteins VP1, VP2, and VP3. These proteins are often found in a ratio of 1:1:10 respectively, but may be in varied ratios, and are all derived from the right-hand ORF. The VP1, VP2 and VP3 capsid proteins differ from each other by the use of alternative splicing and an unusual start codon. Deletion analysis has shown that removal or alteration of VP1 which is translated from an alternatively spliced message results in a reduced yield of infectious particles. Mutations within the VP3 coding region result in the failure to produce any single-stranded progeny DNA or infectious particles.

An AAV particle is a viral particle comprising an AAV capsid. In certain embodiments, the genome of an AAV particle encodes one, two or all VP1, VP2 and VP3 polypeptides.

The genome of most native AAVs often contain two open reading frames (ORFs), sometimes referred to as a left ORF and a right ORF. The left ORF often encodes the non-structural Rep proteins, Rep 40, Rep 52, Rep 68 and Rep 78, which are involved in regulation of replication and transcription in addition to the production of single-stranded progeny genomes. Two of the Rep proteins have been associated with the preferential integration of AAV genomes into a region of the q arm of human chromosome 19. Rep68/78 have been shown to possess NTP binding activity as well as DNA and RNA helicase activities. Some Rep proteins possess a nuclear localization signal as well as several potential phosphorylation sites. In certain embodiments the genome of an AAV (e.g., an rAAV) encodes some or all of the Rep proteins. In certain embodiments the genome of an AAV (e.g., an rAAV) does not encode the Rep proteins. In certain embodiments one or more of the Rep proteins can be delivered in trans and are therefore not included in an AAV particle comprising a nucleic acid encoding a polypeptide.

The ends of the AAV genome comprise short inverted terminal repeats (ITR) which have the potential to fold into T-shaped hairpin structures that serve as the origin of viral DNA replication. Accordingly, the genome of an AAV comprises one or more (e.g., a pair of) ITR sequences that flank a single stranded viral DNA genome. The ITR sequences often have a length of about 145 bases each. Within the ITR region, two elements have been described which are believed to be central to the function of the ITR, a GAGC repeat motif and the terminal resolution site (trs). The repeat motif has been shown to bind Rep when the ITR is in either a linear or hairpin conformation. This binding is thought to position Rep68/78 for cleavage at the trs which occurs in a site- and strand-specific manner. In addition to their role in replication, these two elements appear to be central to viral integration. Contained within the chromosome 19 integration locus is a Rep binding site with an adjacent trs. These elements have been shown to be functional and necessary for locus specific integration.

In certain embodiments, an AAV (e.g., a rAAV) comprises two ITRs. In certain embodiments, an AAV (e.g., a rAAV) comprises a pair of ITRs. In certain embodiments, an AAV (e.g., a rAAV) comprises a pair of ITRs that flank (i.e., are at each 5′ and 3′ end) of a nucleic acid sequence that at least encodes a polypeptide having function or activity.

An AAV vector (e.g., rAAV vector) can be packaged and is referred to herein as an “AAV particle” for subsequent infection (transduction) of a cell, ex vivo, in vitro or in vivo. Where a recombinant AAV vector is encapsulated or packaged into an AAV particle, the particle can also be referred to as a “rAAV particle.” In certain embodiments, an AAV particle is a rAAV particle. A rAAV particle often comprises a rAAV vector, or a portion thereof. A rAAV particle can be one or more rAAV particles (e.g., a plurality of AAV particles). rAAV particles typically comprise proteins that encapsulate or package the rAAV vector genome (e.g., capsid proteins). It is noted that reference to a rAAV vector can also be used to reference a rAAV particle.

Any suitable AAV particle (e.g., rAAV particle) can be used for a method or use herein. A rAAV particle, and/or genome comprised therein, can be derived from any suitable serotype or strain of AAV. A rAAV particle, and/or genome comprised therein, can be derived from two or more serotypes or strains of AAV. Accordingly, a rAAV can comprise proteins and/or nucleic acids, or portions thereof, of any serotype or strain of AAV, wherein the AAV particle is suitable for infection and/or transduction of a mammalian cell. Non-limiting examples of AAV serotypes include AAV1, AAV2, AAV3, AAV4, AAV5, AAV6, AAV7, AAV8, AAV9, AAV10, AAV11, AAV12, AAV-rh74, AAV-rh10 and AAV-2i8.

In certain embodiments a plurality of rAAV particles comprises particles of, or derived from, the same strain or serotype (or subgroup or variant). In certain embodiments a plurality of rAAV particles comprise a mixture of two or more different rAAV particles (e.g., of different serotypes and/or strains).

As used herein, the term “serotype” is a distinction used to refer to an AAV having a capsid that is serologically distinct from other AAV serotypes. Serologic distinctiveness is determined on the basis of the lack of cross-reactivity between antibodies to one AAV as compared to another AAV. Such cross-reactivity differences are usually due to differences in capsid protein sequences/antigenic determinants (e.g., due to VP1, VP2, and/or VP3 sequence differences of AAV serotypes). Despite the possibility that AAV variants including capsid variants may not be serologically distinct from a reference AAV or other AAV serotype, they differ by at least one nucleotide or amino acid residue compared to the reference or other AAV serotype.

In certain embodiments, a rAAV particle excludes certain serotypes. In one embodiment, a rAAV particle is not an AAV4 particle. In certain embodiments, a rAAV particle is antigenically or immunologically distinct from AAV4. Distinctness can be determined by standard methods. For example, ELISA and Western blots can be used to determine whether a viral particle is antigenically or immunologically distinct from AAV4. Furthermore, in certain embodiments a rAAV2 particle retains tissue tropism distinct from AAV4.

In certain embodiments, a rAAV vector based upon a first serotype genome corresponds to the serotype of one or more of the capsid proteins that package the vector. For example, the serotype of one or more AAV nucleic acids (e.g., ITRs) that comprises the AAV vector genome corresponds to the serotype of a capsid that comprises the rAAV particle.

In certain embodiments, a rAAV vector genome can be based upon an AAV (e.g., AAV2) serotype genome distinct from the serotype of one or more of the AAV capsid proteins that package the vector. For example, a rAAV vector genome can comprise AAV2 derived nucleic acids (e.g., ITRs), whereas at least one or more of the three capsid proteins are derived from a different serotype, e.g., an AAV1, AAV3, AAV4, AAV5, AAV6, AAV7, AAV8, AAV9, AAV10, AAV11, AAV12, Rh10, Rh74 or AAV-2i8 serotype or variant thereof.

In certain embodiments, a rAAV particle or a vector genome thereof related to a reference serotype has a polynucleotide, polypeptide or subsequence thereof that comprises or consists of a sequence at least 60% or more (e.g., 65%, 70%, 75%, 80%, 85%, 90%, 95%, 96%, 97%, 98%, 99%, 99.1%, 99.2%, 99.3%, 99.4%, 99.5%, etc.) identical to a polynucleotide, polypeptide or subsequence of an AAV1, AAV2, AAV3, AAV4, AAV5, AAV6, AAV7, AAV8, AAV9, AAV10, AAV11, AAV12, Rh10, Rh74 or AAV-2i8 particle. In particular embodiments, a rAAV particle or a vector genome thereof related to a reference serotype has a capsid or ITR sequence that comprises or consists of a sequence at least 60% or more (e.g., 65%, 70%, 75%, 80%, 85%, 90%, 95%, 96%, 97%, 98%, 99%, 99.1%, 99.2%, 99.3%, 99.4%, 99.5%, etc.) identical to a capsid or ITR sequence of an AAV1, AAV2, AAV3, AAV4, AAV5, AAV6, AAV7, AAV8, AAV9, AAV10, AAV11, AAV12, Rh10, Rh74 or AAV-2i8 serotype.

In certain embodiments, a method herein comprises use, administration or delivery of a rAAV1, rAAV2, rAAV3, rAAV4, rAAV5, rAAV6, rAAV7, rAAV8, rAAV9, rAAV10, rAAV11, rAAV12, rRh10, rRh74 or rAAV-2i8 particle.

In certain embodiments, a method herein comprises use, administration or delivery of a rAAV2 particle. In certain embodiments a rAAV2 particle comprises an AAV2 capsid. In certain embodiments a rAAV2 particle comprises one or more capsid proteins (e.g., VP1, VP2 and/or VP3) that are at least 60%, 65%, 70%, 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to a corresponding capsid protein of a native or wild-type AAV2 particle. In certain embodiments a rAAV2 particle comprises VP1, VP2 and VP3 capsid proteins that are at least 75% or more identical, e.g., 80%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to a corresponding capsid protein of a native or wild-type AAV2 particle. In certain embodiments, a rAAV2 particle is a variant of a native or wild-type AAV2 particle. In some aspects, one or more capsid proteins of an AAV2 variant have 1, 2, 3, 4, 5, 5-10, 10-15, 15-20 or more amino acid substitutions compared to capsid protein(s) of a native or wild-type AAV2 particle.

In certain embodiments a rAAV9 particle comprises an AAV9 capsid. In certain embodiments a rAAV9 particle comprises one or more capsid proteins (e.g., VP1, VP2 and/or VP3) that are at least 60%, 65%, 70%, 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to a corresponding capsid protein of a native or wild-type AAV9 particle. In certain embodiments a rAAV9 particle comprises VP1, VP2 and VP3 capsid proteins that are at least 75% or more identical, e.g., 80%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to a corresponding capsid protein of a native or wild-type AAV9 particle. In certain embodiments, a rAAV9 particle is a variant of a native or wild-type AAV9 particle. In some aspects, one or more capsid proteins of an AAV9 variant have 1, 2, 3, 4, 5, 5-10, 10-15, 15-20 or more amino acid substitutions compared to capsid protein(s) of a native or wild-type AAV9 particle.

In certain embodiments, a rAAV particle comprises one or two ITRs (e.g., a pair of ITRs) that are at least 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to corresponding ITRs of a native or wild-type AAV1, AAV2, AAV3, AAV4, AAV5, AAV6, AAV7, AAV8, AAV9, AAV10, AAV11, AAV12, AAV-rh74, AAV-rh10 or AAV-2i8, as long as they retain one or more desired ITR functions (e.g., ability to form a hairpin, which allows DNA replication; integration of the AAV DNA into a host cell genome; and/or packaging, if desired).

In certain embodiments, a rAAV2 particle comprises one or two ITRs (e.g., a pair of ITRs) that are at least 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to corresponding ITRs of a native or wild-type AAV2 particle, as long as they retain one or more desired ITR functions (e.g., ability to form a hairpin, which allows DNA replication; integration of the AAV DNA into a host cell genome; and/or packaging, if desired).

In certain embodiments, a rAAV9 particle comprises one or two ITRs (e.g., a pair of ITRs) that are at least 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to corresponding ITRs of a native or wild-type AAV2 particle, as long as they retain one or more desired ITR functions (e.g., ability to form a hairpin, which allows DNA replication; integration of the AAV DNA into a host cell genome; and/or packaging, if desired).

A rAAV particle can comprise an ITR having any suitable number of “GAGC” repeats. In certain embodiments an ITR of an AAV2 particle comprises 1, 2, 3, 4, 5, 6, 7, 8, 9 or 10 or more “GAGC” repeats. In certain embodiments a rAAV2 particle comprises an ITR comprising three “GAGC” repeats. In certain embodiments a rAAV2 particle comprises an ITR which has less than four “GAGC” repeats. In certain embodiments a rAAV2 particle comprises an ITR which has more than four “GAGC” repeats. In certain embodiments an ITR of a rAAV2 particle comprises a Rep binding site wherein the fourth nucleotide in the first two “GAGC” repeats is a C rather than a T.

Exemplary suitable length of DNA can be incorporated in rAAV vectors for packaging/encapsulation into a rAAV particle can about 5 kilobases (kb) or less. In particular, embodiments, length of DNA is less than about 5 kb, less than about 4.5 kb, less than about 4 kb, less than about 3.5 kb, less than about 3 kb, or less than about 2.5 kb.

rAAV vectors that include a nucleic acid sequence that directs the expression of an RNAi or polypeptide can be generated using suitable recombinant techniques known in the art (e.g., see Sambrook et al., 1989). Recombinant AAV vectors are typically packaged into transduction-competent AAV particles and propagated using an AAV viral packaging system. A transduction-competent AAV particle is capable of binding to and entering a mammalian cell and subsequently delivering a nucleic acid cargo (e.g., a heterologous gene) to the nucleus of the cell. Thus, an intact rAAV particle that is transduction-competent is configured to transduce a mammalian cell. A rAAV particle configured to transduce a mammalian cell is often not replication competent, and requires additional protein machinery to self-replicate. Thus, a rAAV particle that is configured to transduce a mammalian cell is engineered to bind and enter a mammalian cell and deliver a nucleic acid to the cell, wherein the nucleic acid for delivery is often positioned between a pair of AAV ITRs in the rAAV genome.

Suitable host cells for producing transduction-competent AAV particles include but are not limited to microorganisms, yeast cells, insect cells, and mammalian cells that can be, or have been, used as recipients of a heterologous rAAV vectors. Cells from the stable human cell line, HEK293 (readily available through, e.g., the American Type Culture Collection under Accession Number ATCC CRL1573) can be used. In certain embodiments a modified human embryonic kidney cell line (e.g., HEK293), which is transformed with adenovirus type-5 DNA fragments, and expresses the adenoviral Ela and E1b genes is used to generate recombinant AAV particles. The modified HEK293 cell line is readily transfected, and provides a particularly convenient platform in which to produce rAAV particles. Methods of generating high titer AAV particles capable of transducing mammalian cells are known in the art. For example, AAV particle can be made as set forth in Wright, 2008 and Wright, 2009.

In certain embodiments, AAV helper functions are introduced into the host cell by transfecting the host cell with an AAV helper construct either prior to, or concurrently with, the transfection of an AAV expression vector. AAV helper constructs are thus sometimes used to provide at least transient expression of AAV rep and/or cap genes to complement missing AAV functions necessary for productive AAV transduction. AAV helper constructs often lack AAV ITRs and can neither replicate nor package themselves. These constructs can be in the form of a plasmid, phage, transposon, cosmid, virus, or virion. A number of AAV helper constructs have been described, such as the commonly used plasmids pAAV/Ad and pIM29+45 which encode both Rep and Cap expression products. A number of other vectors are known which encode Rep and/or Cap expression products.

3. Retrovirus

Viral vectors for use as a delivered agent in the methods, compositions and uses herein include a retroviral vector (see e.g., Miller (1992) Nature, 357:455-460). Retroviral vectors are well suited for delivering nucleic acid into cells because of their ability to deliver an unrearranged, single copy gene into a broad range of rodent, primate and human somatic cells. Retroviral vectors integrate into the genome of host cells. Unlike other viral vectors, they only infect dividing cells.

Retroviruses are RNA viruses such that the viral genome is RNA. When a host cell is infected with a retrovirus, the genomic RNA is reverse transcribed into a DNA intermediate, which is integrated into the chromosomal DNA of infected cells. This integrated DNA intermediate is referred to as a provirus. Transcription of the provirus and assembly into infectious virus occurs in the presence of an appropriate helper virus or in a cell line containing appropriate sequences permitting encapsulation without coincident production of a contaminating helper virus. A helper virus is not required for the production of the recombinant retrovirus if the sequences for encapsulation are provided by co-transfection with appropriate vectors.

The retroviral genome and the proviral DNA have three genes: the gag, the pol and the env, which are flanked by two long terminal repeat (LTR) sequences. The gag gene encodes the internal structural (matrix, capsid, and nucleocapsid) proteins and the env gene encodes viral envelope glycoproteins. The pol gene encodes products that include the RNA-directed DNA polymerase reverse transcriptase that transcribes the viral RNA into double-stranded DNA, integrase that integrate the DNA produced by reverse transcriptase into host chromosomal DNA, and protease that acts to process the encoded gag and pol genes. The 5′ and 3′ LTRs serve to promote transcription and polyadenylation of the virion RNAs. The LTR contains all other cis-acting sequences necessary for viral replication.

Retroviral vectors are described by Coffin et al., Retroviruses, Cold Spring Harbor Laboratory Press (1997). Exemplary of a retrovirus is Moloney murine leukemia virus (MMLV) or the murine stem cell virus (MSCV). Retroviral vectors can be replication-competent or replication-defective. Typically, a retroviral vector is replication-defective in which the coding regions for genes necessary for additional rounds of virion replication and packaging are deleted or replaced with other genes. Consequently, the viruses are not able to continue their typical lytic pathway once an initial target cell is infected. Such retroviral vectors, and the necessary agents to produce such viruses (e.g., packaging cell line) are commercially available (see, e.g., retroviral vectors and systems available from Clontech, such as Catalog number 634401, 631503, 631501, and others, Clontech, Mountain View, Calif.).

Such retroviral vectors can be produced as delivered agents by replacing the viral genes required for replication with the nucleic acid molecule to be delivered. The resulting genome contains an LTR at each end with the desired gene or genes in between. Methods of producing retrovirus are known to one of skill in the art (see, e.g., International published PCT Application No. WO1995/026411). The retroviral vector can be produced in a packaging cell line containing a helper plasmid or plasmids. The packaging cell line provides the viral proteins required for capsid production and the virion maturation of the vector (e.g., gag, pol and env genes). Typically, at least two separate helper plasmids (separately containing the gag and pol genes; and the env gene) are used so that recombination between the vector plasmid cannot occur. For example, the retroviral vector can be transferred into a packaging cell line using standard methods of transfection, such as calcium phosphate mediated transfection. Packaging cell lines are well known to one of skill in the art, and are commercially available. An exemplary packaging cell line is GP2-293 packaging cell line (Catalog Numbers 631505, 631507, 631512, Clontech). After sufficient time for virion production, the virus is harvested. If desired, the harvested virus can be used to infect a second packaging cell line, for example, to produce a virus with varied host tropism. The end result is a replicative incompetent recombinant retrovirus that includes the nucleic acid of interest but lacks the other structural genes such that a new virus cannot be formed in the host cell.

References illustrating the use of retroviral vectors in gene therapy include: Clowes et al., (1994) J. Clin. Invest. 93:644-651; Kiem et al., (1994) Blood 83:1467-1473; Salmons and Gunzberg (1993) Human Gene Therapy 4:129-141; Grossman and Wilson (1993) Curr. Opin. in Genetics and Devel. 3:110-114; Sheridan (2011) Nature Biotechnology, 29:121; Cassani et al. (2009) Blood, 114:3546-3556.

4. Lentivirus

Lentiviruses are complex retroviruses, which, in addition to the common retroviral genes gag, pol, and env, contain other genes with regulatory or structural function. The higher complexity enables the virus to modulate its life cycle, as in the course of latent infection. Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe. Lentiviral vectors are well known in the art (see, e.g., U.S. Pat. Nos. 6,013,516 and 5,994,136).

Recombinant lentiviral vectors are capable of infecting non-dividing cells and can be used for both in vivo and ex vivo gene transfer and expression of nucleic acid sequences. For example, a recombinant lentivirus capable of infecting a non-dividing cell, wherein a suitable host cell is transfected with two or more vectors carrying the packaging functions, namely gag, pol and env, as well as rev and tat, is described in U.S. Pat. No. 5,994,136, incorporated herein by reference.

The lentiviral genome and the proviral DNA have the three genes found in retroviruses: gag, pol and env, which are flanked by two long terminal repeat (LTR) sequences. The gag gene encodes the internal structural (matrix, capsid and nucleocapsid) proteins; the pol gene encodes the RNA-directed DNA polymerase (reverse transcriptase), a protease and an integrase; and the env gene encodes viral envelope glycoproteins. The 5′ and 3′ LTRs serve to promote transcription and polyadenylation of the virion RNAs. The LTR contains all other cis-acting sequences necessary for viral replication. Lentiviruses have additional genes including vif, vpr, tat, rev, vpu, nef and vpx.

Adjacent to the 5′ LTR are sequences necessary for reverse transcription of the genome (the tRNA primer binding site) and for efficient encapsidation of viral RNA into particles (the Psi site). If the sequences necessary for encapsidation (or packaging of retroviral RNA into infectious virions) are missing from the viral genome, the cis defect prevents encapsidation of genomic RNA. However, the resulting mutant remains capable of directing the synthesis of all virion proteins.

5. Other Viral Vectors

The development and utility of viral vectors for gene delivery is constantly improving and evolving. Other viral vectors such as poxvirus; e.g., vaccinia virus (Gnant et al., 1999; Gnant et al., 1999), alpha virus; e.g., sindbis virus, Semliki forest virus (Lundstrom, 1999), reovirus (Coffey et al., 1998) and influenza A virus (Neumann et al., 1999) are contemplated for use in the present disclosure and may be selected according to the requisite properties of the target system.

6. Chimeric Viral Vectors

Chimeric or hybrid viral vectors are being developed for use in therapeutic gene delivery and are contemplated for use in the present disclosure. Chimeric poxviral/retroviral vectors (Holzer et al., 1999), adenoviral/retroviral vectors (Feng et al., 1997; Bilbao et al., 1997; Caplen et al., 2000) and adenoviral/adeno-associated viral vectors (Fisher et al., 1996; U.S. Pat. No. 5,871,982) have been described. These “chimeric” viral gene transfer systems can exploit the favorable features of two or more parent viral species. For example, Wilson et al., provide a chimeric vector construct which comprises a portion of an adenovirus, AAV 5′ and 3′ ITR sequences and a selected transgene, described below (U.S. Pat. No. 5,871,983, specifically incorporate herein by reference).

### IV. Methods of Administering Nucleic Acid Expression Cassettes or Engineered Cells

Any suitable cell or mammal can be administered or treated by a method or use described herein. Typically, a mammal is in need of a method described herein, that is suspected of having or expressing an abnormal or aberrant protein that is associated with a disease state.

Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In certain embodiments a mammal is a human. In certain embodiments a mammal is a non-rodent mammal (e.g., human, pig, goat, sheep, horse, dog, or the like). In certain embodiments a non-rodent mammal is a human. A mammal can be any age or at any stage of development (e.g., an adult, teen, child, infant, or a mammal in utero). A mammal can be male or female. In certain embodiments a mammal can be an animal disease model, for example, animal models having or expressing an abnormal or aberrant protein that is associated with a disease state or animal models with insufficient expression of a protein, which causes a disease state.

Mammals (subjects) treated by a method or composition described herein include adults (18 years or older) and children (less than 18 years of age). Adults include the elderly. Representative adults are 50 years or older. Children range in age from 1-2 years old, or from 2-4, 4-6, 6-18, 8-10, 10-12, 12-15 and 15-18 years old. Children also include infants. Infants typically range from 1-12 months of age.

In certain embodiments, a method includes administering a plurality of viral particles or engineered cells to a mammal as set forth herein, where severity, frequency, progression or time of onset of one or more symptoms of a disease state. In certain embodiments, a method includes administering a plurality of viral particles or engineered cells to a mammal to stabilize, delay or prevent worsening, or progression, or reverse and adverse symptom of a disease state.

In some embodiments, viral and non-viral based gene transfer methods can be used to introduce nucleic acids in mammalian cells or target tissues. Such methods can be used to administer nucleic acids encoding therapeutic proteins or components of a CRISPR system to cells in culture or in a host organism. Non-viral vector delivery systems include DNA plasmids, RNA (e.g., a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell.

Methods of non-viral delivery of nucleic acids include exosomes, lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection reagents are sold commercially (e.g., Transfectam™ and Lipofectin™). Delivery can be to cells (e.g., in vitro or ex vivo administration) or target tissues (e.g., in vivo administration).

In some embodiments, delivery is via the use of RNA or DNA viral based systems for the delivery of nucleic acids. Viral vectors in some aspects may be administered directly to subjects (in vivo) or they can be used to treat cells in vitro or ex vivo, and then administered to subjects. Viral-based systems in some embodiments include retroviral, lentivirus, adenoviral, adeno-associated and herpes simplex virus vectors for gene transfer.

The gene expression systems herein can be delivered ex vivo to cells, which are then encapsulated and implanted in order to deliver the therapeutic product to a subject. For example, cells isolated from a subject or a donor introduced with an exogenous heterologous nucleic acid can be delivered directly to a subject by implantation of encapsulated cells. The advantage of implantation of encapsulated cells is that the immune response to the cells is reduced by the encapsulation. Thus, provided herein is a method of administering a genetically modified cell or cells to a subject. The number of cells that are delivered depends on the desired effect, the particular nucleic acid, the subject being treated and other similar factors, and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, or fetal liver. For example, the genetically modified cells can be pluripotent or totipotent stem cells (including induced pluripotent stem cells) or can be embryonic, fetal, or fully differentiated cells. The genetically modified cells can be cells from the same subject or can be cells from the same or different species as the recipient subject. In a preferred example, the cell used for gene therapy is autologous to the subject. Methods of genetically modifying cells and transplanting cells are known in the art.

Typically, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells and can be used provided that the necessary developmental and physiological functions of the recipient cells are not disrupted. In particular examples, the method is one that permits stable transfer of the nucleic acid to the cell, so that the nucleic acid is expressible by the cell and heritable and expressible by its cell progeny.

Encapsulation can be performed using a hydrogel capsule, for example, an alginate microcapsule coated with an alginate/polylysine complex. Hydrogel microcapsules have been extensively investigated for encapsulation of living cells or cell aggregates for tissue engineering and regenerative medicine. In general, capsules are designed to allow facile diffusion of oxygen and nutrients to the encapsulated cells, while releasing the therapeutic proteins secreted by the cells, and to protect the cells from attack by the immune system. One of the most common capsule formulations is based on alginate hydrogels, which can be formed through ionic crosslinking. In a typical process, the cells are first blended with a viscous alginate solution. The cell suspension is then processed into micro-droplets using different methods such as air shear, acoustic vibration or electrostatic droplet formation. The alginate droplet is gelled upon contact with a solution of divalent ions, such as Ca²⁺ or Ba²⁺. In one embodiment, the present disclosure features a hydrogel capsule (e.g., an alginate capsule) comprising a cell bearing a vector described herein.

### V. Pharmaceutical Compositions

As used herein the term “pharmaceutically acceptable” and “physiologically acceptable” mean a biologically acceptable composition, formulation, liquid or solid, or mixture thereof, which is suitable for one or more routes of administration, in vivo delivery or contact. A “pharmaceutically acceptable” or “physiologically acceptable” composition is a material that is not biologically or otherwise undesirable, e.g., the material may be administered to a subject without causing substantial undesirable biological effects. Such composition, “pharmaceutically acceptable” and “physiologically acceptable” formulations and compositions can be sterile. Such pharmaceutical formulations and compositions may be used, for example in administering a viral particle or nanoparticle to a subject.

Such formulations and compositions include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Supplementary active compounds (e.g., preservatives, antibacterial, antiviral and antifungal agents) can also be incorporated into the formulations and compositions.

Pharmaceutical compositions typically contain a pharmaceutically acceptable excipient. Such excipients include any pharmaceutical agent that does not itself induce the production of antibodies harmful to the individual receiving the composition, and which may be administered without undue toxicity. Pharmaceutically acceptable excipients include, but are not limited to, sorbitol, Tween80, and liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as surfactants, wetting or emulsifying agents, pH buffering substances, and the like, may be present in such vehicles.

Pharmaceutical compositions can be formulated to be compatible with a particular route of administration or delivery, as set forth herein or known to one of skill in the art. Thus, pharmaceutical compositions include carriers, diluents, or excipients suitable for administration or delivery by various routes.

Pharmaceutical forms suitable for injection or infusion of viral particles or engineered cells can include sterile aqueous solutions or dispersions which are adapted for the extemporaneous preparation of sterile injectable or infusible solutions or dispersions, optionally encapsulated in liposomes. In all cases, the ultimate form should be a sterile fluid and stable under the conditions of manufacture, use and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Isotonic agents, for example, sugars, buffers or salts (e.g., sodium chloride) can be included. Prolonged absorption of injectable compositions can be brought about by the use in the compositions of agents delaying absorption, for example, aluminum monostearate and gelatin.

Solutions or suspensions of viral particles or engineered cells can optionally include one or more of the following components: a sterile diluent such as water for injection, saline solution, such as phosphate buffered saline (PBS), artificial CSF, a surfactants, fixed oils, a polyol (for example, glycerol, propylene glycol, and liquid polyethylene glycol, and the like), glycerin, or other synthetic solvents; antibacterial and antifungal agents such as parabens, chlorobutanol, phenol, ascorbic acid, and the like; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose.

Viral particles, engineered cells, and their compositions may be formulated in dosage unit form for ease of administration and uniformity of dosage. Dosage unit form as used herein refers to physically discrete units suited as unitary dosages for an individual to be treated; each unit containing a predetermined quantity of active compound calculated to produce the desired therapeutic effect in association with the required pharmaceutical carrier. The dosage unit forms are dependent upon the number of viral particles or nanoparticles believed necessary to produce the desired effect(s). The amount necessary can be formulated in a single dose, or can be formulated in multiple dosage units. The dose may be adjusted to a suitable viral particle or nanoparticle concentration, optionally combined with an anti-inflammatory agent, and packaged for use.

In one embodiment, pharmaceutical compositions will include sufficient genetic material to provide a therapeutically effective amount, i.e., an amount sufficient to reduce or ameliorate symptoms or an adverse effect of a disease state in question or an amount sufficient to confer the desired benefit.

A “unit dosage form” as used herein refers to physically discrete units suited as unitary dosages for the subject to be treated; each unit containing a predetermined quantity optionally in association with a pharmaceutical carrier (excipient, diluent, vehicle or filling agent) which, when administered in one or more doses, is calculated to produce a desired effect (e.g., prophylactic or therapeutic effect). Unit dosage forms may be within, for example, ampules and vials, which may include a liquid composition, or a composition in a freeze-dried or lyophilized state; a sterile liquid carrier, for example, can be added prior to administration or delivery in vivo. Individual unit dosage forms can be included in multi-dose kits or containers. Thus, for example, viral particles, nanoparticles, and pharmaceutical compositions thereof can be packaged in single or multiple unit dosage form for ease of administration and uniformity of dosage.

Formulations containing viral particles or engineered cells typically contain an effective amount, the effective amount being readily determined by one skilled in the art. The viral particles may typically range from about 1% to about 95% (w/w) of the composition, or even higher if suitable. The quantity to be administered depends upon factors such as the age, weight and physical condition of the mammal or the human subject considered for treatment. Effective dosages can be established by one of ordinary skill in the art through routine trials establishing dose response curves.

### VI. Definitions

The terms “polynucleotide,” “nucleic acid,” and “transgene” are used interchangeably herein to refer to all forms of nucleic acid, including deoxyribonucleic acid (DNA) and polymers thereof. Polynucleotides can be naturally occurring, synthetic, and intentionally modified or altered polynucleotides. Polynucleotides can be single stranded or double stranded, linear or circular, and can be of any suitable length. In discussing polynucleotides, a sequence or structure of a particular polynucleotide may be described herein according to the convention of providing the sequence in the 5′ to 3′ direction.

A nucleic acid encoding a polypeptide often comprises an open reading frame that encodes the polypeptide. Unless otherwise indicated, a particular nucleic acid sequence also includes degenerate codon substitutions.

Nucleic acids can include one or more expression control or regulatory elements operably linked to the open reading frame, where the one or more regulatory elements are configured to direct the transcription and translation of the polypeptide encoded by the open reading frame in a mammalian cell. Non-limiting examples of expression control/regulatory elements include transcription initiation sequences (e.g., promoters, enhancers, a TATA box, and the like), translation initiation sequences, mRNA stability sequences, poly A sequences, secretory sequences, and the like. Expression control/regulatory elements can be obtained from the genome of any suitable organism.

A “promoter” refers to a nucleotide sequence, usually upstream (5′) of a coding sequence, which directs and/or controls the expression of the coding sequence by providing the recognition for RNA polymerase and other factors required for proper transcription. “Promoter” includes a minimal promoter that is a short DNA sequence comprised of a TATA-box and optionally other sequences that serve to specify the site of transcription initiation, to which regulatory elements are added for control of expression.

Promoters may be derived in their entirety from a native gene, or be composed of different elements derived from different elements found in nature, or even be comprised of synthetic DNA segments. A promoter may comprise DNA sequences that are involved in the binding of protein factors that modulate/control effectiveness of transcription initiation in response to stimuli, physiological conditions, or developmental conditions.

Non-limiting examples include SV40 early promoter, mouse mammary tumor virus LTR promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, pol II promoters, pol III promoters, synthetic promoters, hybrid promoters, and the like. Exemplary constitutive promoters include the promoters for the following genes which encode certain constitutive or “housekeeping” functions: hypoxanthine phosphoribosyl transferase (HPRT), dihydrofolate reductase (DHFR), adenosine deaminase, phosphoglycerol kinase (PGK), pyruvate kinase, phosphoglycerol mutase, the actin promoter, and other constitutive promoters known to those of skill in the art. In addition, many viral promoters function constitutively in eukaryotic cells. These include: the early and late promoters of SV40; the long terminal repeats (LTRs) of Moloney Leukemia Virus and other retroviruses; and the thymidine kinase promoter of Herpes Simplex Virus, among many others. Accordingly, any of the above-referenced constitutive promoters can be used to control transcription of a heterologous gene insert.

A “transgene” is used herein to conveniently refer to a nucleic acid sequence/polynucleotide that is intended or has been introduced into a cell or organism. Transgenes include any nucleic acid, such as a gene that encodes a Cas9 protein, a gRNA, or a therapeutic polypeptide or protein.

The term “transduce” refers to introduction of a nucleic acid sequence into a cell or host organism by way of a vector (e.g., a viral particle). Introduction of a transgene into a cell by a viral particle is can therefore be referred to as “transduction” of the cell. The transgene may or may not be integrated into genomic nucleic acid of a transduced cell. If an introduced transgene becomes integrated into the nucleic acid (genomic DNA) of the recipient cell or organism, it can be stably maintained in that cell or organism and further passed on to or inherited by progeny cells or organisms of the recipient cell or organism. Finally, the introduced transgene may exist in the recipient cell or host organism extra chromosomally, or only transiently. At least for gene therapy uses and methods, a transduced cell can be in a mammal.

Transgenes under control of inducible promoters are expressed only or to a greater degree, in the presence of an inducing agent. Inducible promoters include responsive elements (REs), which stimulate transcription when their inducing factors are bound. For example, there are REs for serum factors, steroid hormones, retinoic acid and cyclic AMP. Promoters containing a particular RE can be chosen in order to obtain an inducible response and in some cases, the RE itself may be attached to a different promoter, thereby conferring inducibility to the recombinant gene (e.g., a dCas9-Activator protein). Thus, by selecting a suitable promoter (constitutive versus inducible; strong versus weak), it is possible to control both the existence and level of expression of a polypeptide in the genetically modified cell. If the gene encoding the polypeptide is under the control of an inducible promoter, delivery of the polypeptide in situ is triggered by exposing the genetically modified cell in situ to conditions for permitting transcription of the polypeptide.

A nucleic acid/transgene is “operably linked” when it is placed into a functional relationship with another nucleic acid sequence. A nucleic acid operably linked to an expression control element (i.e., a promoter) can also be referred to as an expression cassette.

As used herein, the terms “modify” or “variant” and grammatical variations thereof, mean that a nucleic acid, polypeptide or subsequence thereof deviates from a reference sequence. Modified and variant sequences may therefore have substantially the same, greater or less expression, activity or function than a reference sequence, but at least retain partial activity or function of the reference sequence. A particular type of variant is a mutant protein, which refers to a protein encoded by a gene having a mutation, e.g., a missense or nonsense mutation.

A “nucleic acid variant” refers to a modified sequence which has been genetically altered compared to wild-type. The sequence may be genetically modified without altering the encoded protein sequence. Alternatively, the sequence may be genetically modified to encode a variant protein. A nucleic acid or polynucleotide variant can also refer to a combination sequence which has been codon modified to encode a protein that still retains at least partial sequence identity to a reference sequence, such as wild-type protein sequence, and also has been codon-modified to encode a variant protein. For example, some codons of such a nucleic acid variant will be changed without altering the amino acids of a protein encoded thereby, and some codons of the nucleic acid variant will be changed which in turn changes the amino acids of a protein encoded thereby.

The terms “protein” and “polypeptide” are used interchangeably herein. The “polypeptides” encoded by a “nucleic acid” or “polynucleotide” or “transgene” disclosed herein include partial or full-length native sequences, as with naturally occurring wild-type and functional polymorphic proteins, functional subsequences (fragments) thereof, and sequence variants thereof, so long as the polypeptide retains some degree of function or activity. Accordingly, in methods and uses of the invention, such polypeptides encoded by nucleic acid sequences are not required to be identical to the endogenous protein that is defective, or whose activity, function, or expression is insufficient, deficient or absent in a treated mammal.

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of molecular biology techniques, as, for example, with polymerase chain reaction (PCR) and hybridization techniques. Variant nucleotide sequences also include synthetically derived nucleotide sequences, such as those generated, for example, by using site-directed mutagenesis, which encode the native protein, as well as those that encode a polypeptide having amino acid substitutions. Generally, nucleotide sequence variants of the invention will have at least 40%, 50%, 60%, to 70%, e.g., 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous) nucleotide sequence. In certain embodiments, the variant is biologically functional (i.e., retains 5%, 10%, 15%, 20%, 25%, 30%, 35%, 40%, 45%, 50%, 55%, 60%, 65%, 70%, 75%, 80%, 85%, 90%, 95%, 99% or 100% of activity or function of wild-type).

“Conservative variations” of a particular nucleic acid sequence refers to those nucleic acid sequences that encode identical or essentially identical amino acid sequences. Because of the degeneracy of the genetic code, a large number of functionally identical nucleic acids encode any given polypeptide. For instance, the codons CGT, CGC, CGA, CGG, AGA and AGG all encode the amino acid arginine. Thus, at every position where an arginine is specified by a codon, the codon can be altered to any of the corresponding codons described without altering the encoded protein. Such nucleic acid variations are “silent variations,” which are one species of “conservatively modified variations.” Every nucleic acid sequence described herein that encodes a polypeptide also describes every possible silent variation, except where otherwise noted. One of skill in the art will recognize that each codon in a nucleic acid (except ATG, which is ordinarily the only codon for methionine) can be modified to yield a functionally identical molecule by standard techniques. Accordingly, each “silent variation” of a nucleic acid that encodes a polypeptide is implicit in each described sequence.

The term “substantial identity” of polynucleotide sequences means that a polynucleotide comprises a sequence that has at least 70%, 71%, 72%, 73%, 74%, 75%, 76%, 77%, 78%, or 79%, or at least 80%, 81%, 82%, 83%, 84%, 85%, 86%, 8′7%, 88%, or 89%, or at least 90%, 91%, 92%, 93%, or 94%, or even at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, at least 80%, 90%, or even at least 95%.

The term “substantial identity” in the context of a polypeptide indicates that a polypeptide comprises a sequence with at least 70%, 71%, 72%, 73%, 74%, 75%, 76%, 77%, 78%, or 79%, or 80%, 81%, 82%, 83%, 84%, 85%, 86%, 87%, 88%, or 89%, or at least 90%, 91%, 92%, 93%, or 94%, or even, 95%, 96%, 97%, 98% or 99%, sequence identity to the reference sequence over a specified comparison window. An indication that two polypeptide sequences are identical is that one polypeptide is immunologically reactive with antibodies raised against the second polypeptide. Thus, a polypeptide is identical to a second polypeptide, for example, where the two peptides differ only by a conservative substitution.

The terms “treat” and “treatment” refer to both therapeutic treatment and prophylactic or preventative measures, wherein the object is to prevent, inhibit, reduce, or decrease an undesired physiological change or disorder, such as the development, progression or worsening of the disorder. For purposes of this invention, beneficial or desired clinical results include, but are not limited to, alleviation of symptoms, diminishment of extent of disease, stabilizing a (i.e., not worsening or progressing) symptom or adverse effect of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those predisposed (e.g., as determined by a genetic assay).

The term “subject” generally refers to a human, but also may include other mammals such as horses, cows, sheep, pigs, mice, rats, dogs, cats, and primates. In an embodiment, the subject is a human. In another embodiment, the subject is a mammal who exhibits one or more symptoms characteristic of a disorder. In another embodiment, the subject is a human who exhibits one or more symptoms characteristic of a disorder. In another embodiment, the subject is a patient. However, the term subject does not require one to have any particular status or relationship with respect to a hospital, clinic, research facility, or physician (e.g., as an admitted patient, a study participant, or the like).

### VII. Kits

The invention provides kits with packaging material and one or more components therein. A kit typically includes a label or packaging insert including a description of the components or instructions for use in vitro, in vivo, or ex vivo, of the components therein. A kit can contain a collection of such components, e.g., a nucleic acid, recombinant vector, viral particles, and optionally a second active agent, such as another compound, agent, drug or composition.

A kit refers to a physical structure housing one or more components of the kit. Packaging material can maintain the components sterilely, and can be made of material commonly used for such purposes (e.g., paper, corrugated fiber, glass, plastic, foil, ampules, vials, tubes, etc.).

Labels or inserts can include identifying information of one or more components therein, dose amounts, clinical pharmacology of the active ingredient(s) including mechanism of action, pharmacokinetics and pharmacodynamics. Labels or inserts can include information identifying manufacturer, lot numbers, manufacture location and date, expiration dates. Labels or inserts can include information identifying manufacturer information, lot numbers, manufacturer location and date. Labels or inserts can include information on a disease for which a kit component may be used. Labels or inserts can include instructions for the clinician or subject for using one or more of the kit components in a method, use, or treatment protocol or therapeutic regimen. Instructions can include dosage amounts, frequency or duration, and instructions for practicing any of the methods, uses, treatment protocols or prophylactic or therapeutic regimes described herein.

### VIII. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Analysis of Promoters in HEK293T Cells

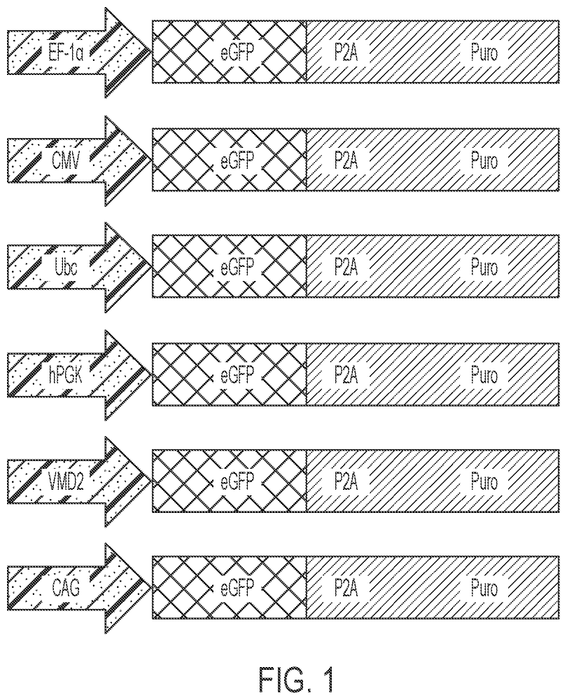

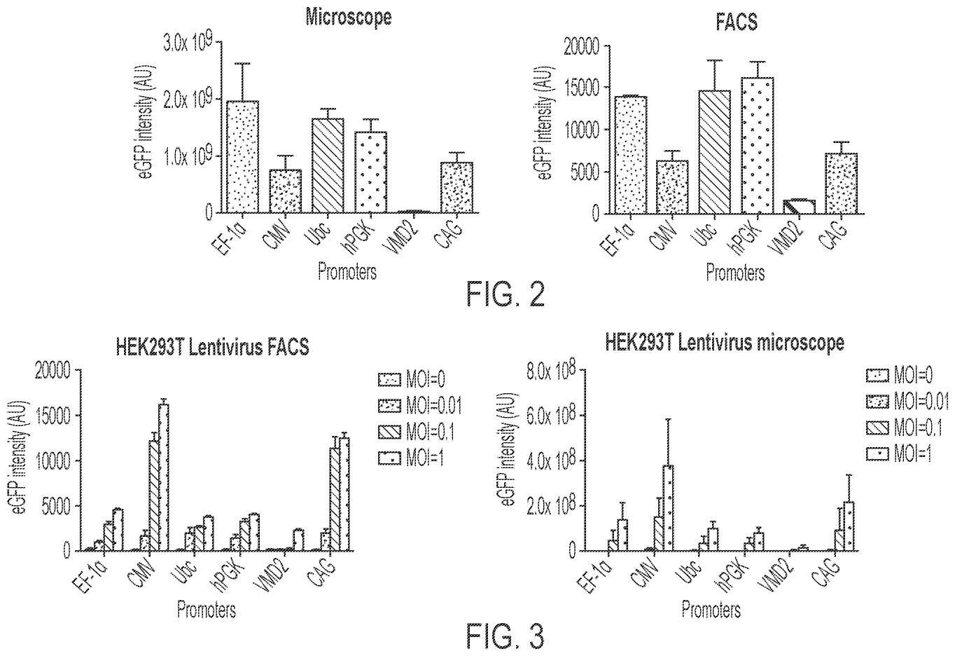

HEK293T cells were transduced with the eGFP gene under the control of different promoters (EF-1α, CMV, Ubc, hPGK, VMD2, and CAG; FIG. 1) using transient transfection. The intensity of eGFP fluorescence was measured by both microscopy and FACS (FIG. 2). The EF-1α, Ubc, hPGK, and CAG promoters strongly worked in HEK293T cells.

In addition, HEK293T cells were transduced with the eGFP gene under the control of different promoters (EF-1α, CMV, Ubc, hPGK, VMD2, and CAG; FIG. 1) using lentivirus. The intensity of eGFP fluorescence was measured by both microscopy and FACS (FIG. 3). Both the CAG and CMV promoters strongly worked in HEK293T cells, but the CMV promoter was slightly better than the CAG promoter.

### Example 2—Analysis of Promoters in ARPE-19 Cells

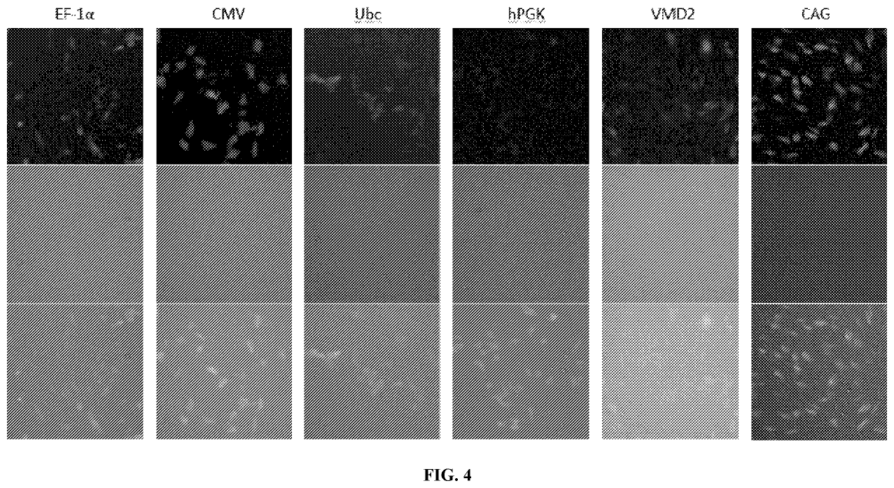

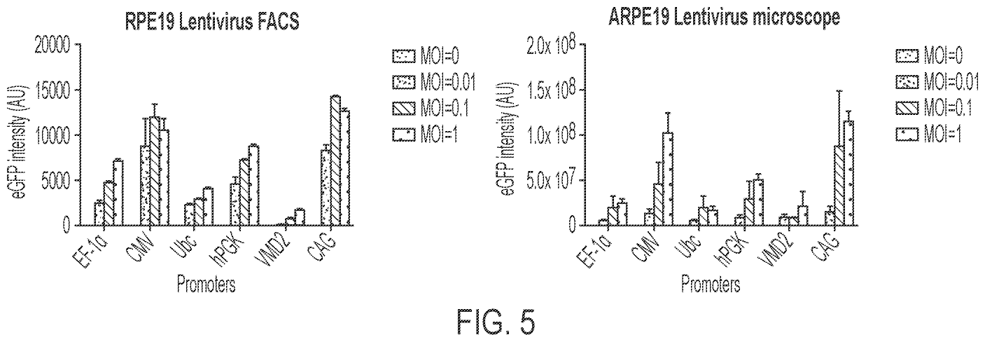

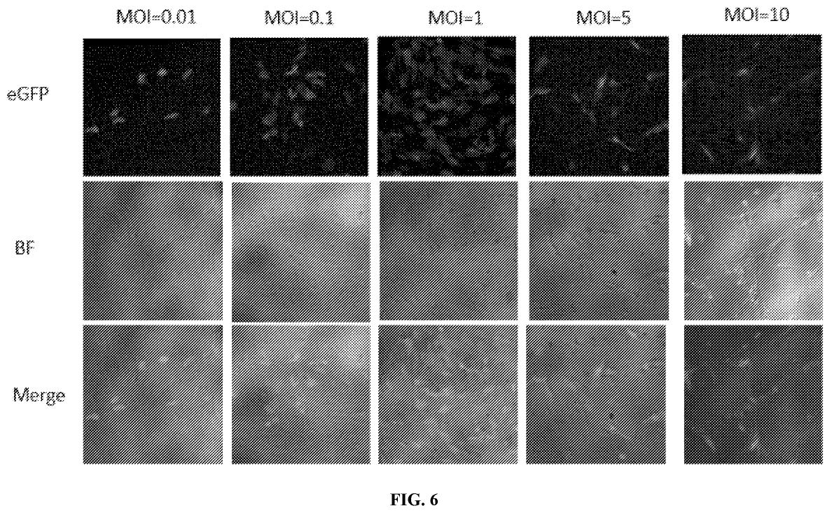

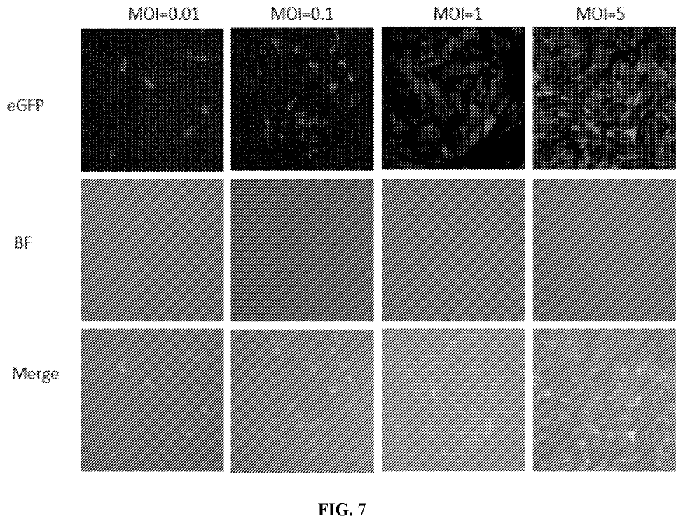

ARPE-19 cells were transduced with the eGFP gene under the control of different promoters (EF-1α, CMV, Ubc, hPGK, VMD2, and CAG; FIG. 1) using lentivirus. The intensity of eGFP fluorescence was measured by both microscopy and FACS (FIG. 4; FIG. 5). Both the CAG and CMV promoters strongly worked in ARPE-19 cells, but the CAG promoter was slightly better than the CMV promoter. However, CAG-driven eGFP expression was more toxic than CMV-driven eGFP expression (FIG. 6; FIG. 7).

### Example 3—Constitutive Expression of Therapeutic Genes in ARPE-19 Cells

While the CAG promoter is strongly activated in ARPE-19 cells, it is prone to epigenetic silencing over time. As such, ARPE-19 cells are engineered with CAG promoter-targeting synthetic transcription factors based upon the CRISPR-dCas9-activator system.

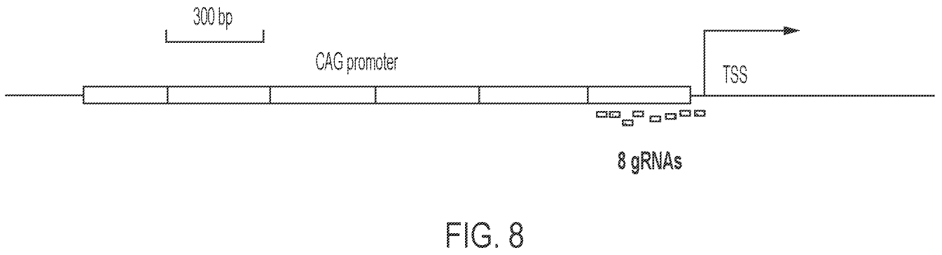

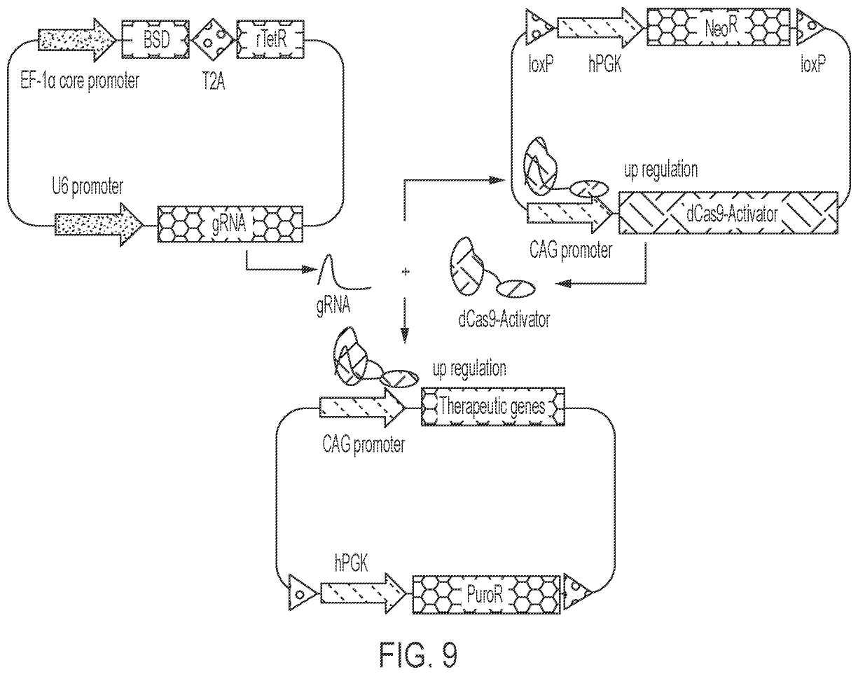

In these engineered cells, both the therapeutic gene and the dCas9-activator gene are driven by the CAG promoter. A gRNA targeting the CAG promoter (FIG. 8) is constitutively expressed by the U6 promoter. In the engineered cells, the gRNA-dCas9-activator complex will target the CAG promoters and activate them, such that both the therapeutic gene and the dCas9-activator are constitutively and highly expressed (FIG. 9).

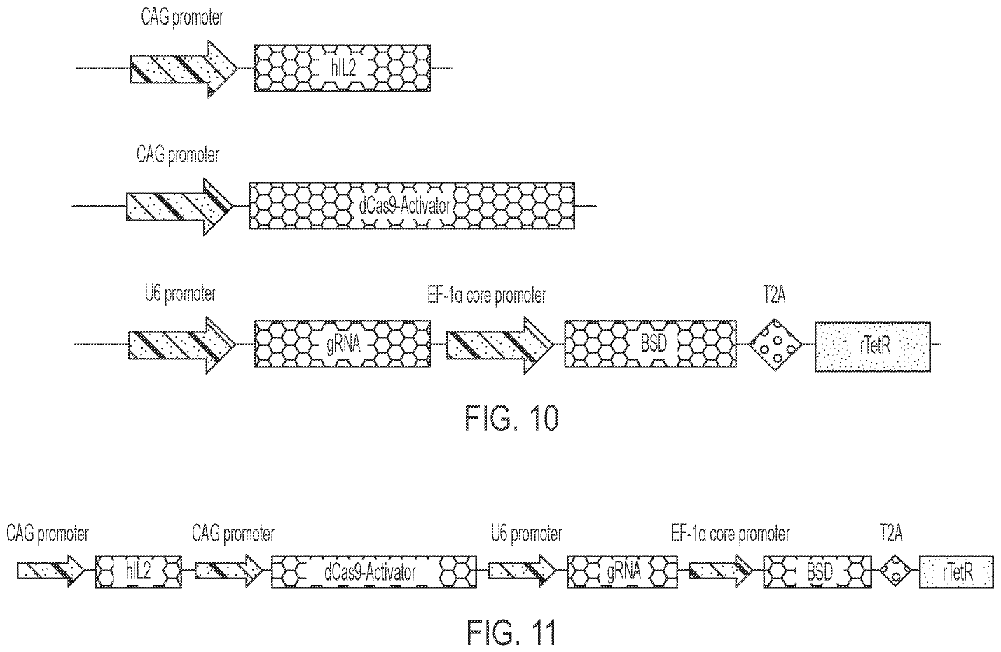

To generate these engineered cells, the cassettes of the CAG-therapeutic gene, CAG-dCas9-activator gene, and U6-gRNA are integrated into the genome of ARPE-19 cells by lentiviral transduction (FIG. 10). In addition, the constructs carrying both the CAG-therapeutic gene cassette and the CAG-dCas9-activator gene cassette will carry antibiotic resistance gene cassettes. This allows the transduced cells to be selected with the corresponding antibiotics. In addition, in order to reduce the toxicity of antibiotic resistance, the antibiotic resistance gene cassettes are flanked by a LoxP site, allowing for the antibiotic resistance gene cassettes to be removed by introducing Cre enzymes into the cells.

As an alternative method to generate these engineered cells, the various component cassettes are integrated into the AAV safe harbor by the AAVS1-targeting HR donor vectors (FIG. 11).

### Example 4—Inducible Expression of Therapeutic Genes in ARPE-19 Cells

Provided here are exemplary methods to establish ARPE-10 cell lines that specifically express therapeutic genes in response to extracellular stimuli. As such, ARPE-19 cells are engineered with minimal promoter-targeting synthetic transcription factors based upon the CRISPR-dCas9-activator system. For this, expression of the therapeutic gene is driven by a minimal promoter (e.g., the minimal CMV promoter) such that the basal expression of the therapeutic gene is very low. However, the minimal promoter can be strongly activated by the dCas9-activator system, the expression of which is controlled by the doxycycline-inducible Tre promoter.

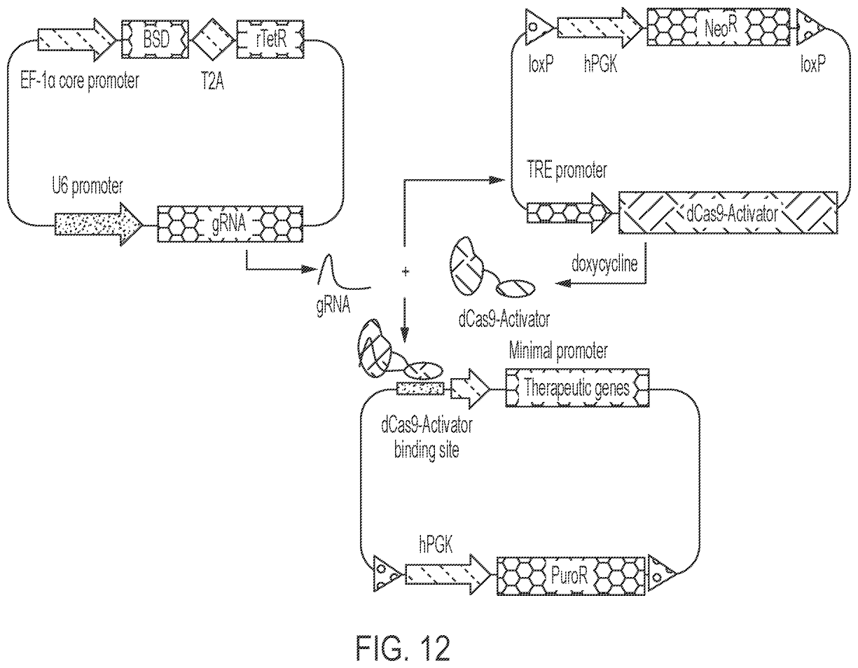

In these engineered cells, the therapeutic gene is driven by a minimal promoter. The dCas9-activator gene is driven by an inducible promoter (e.g., a doxycycline-inducible Tre promoter). A gRNA targeting the minimal promoter is constitutively expressed by the U6 promoter. In the engineered cells, the gRNA-dCas9-activator complex will target the minimal promoter and activate it, such that the therapeutic gene is inducibly expressed. As such, doxycycline will induce the expression of the dCas9-activator, which will then activate the minimal promoter, thereby causing expression of the therapeutic gene (FIG. 12).

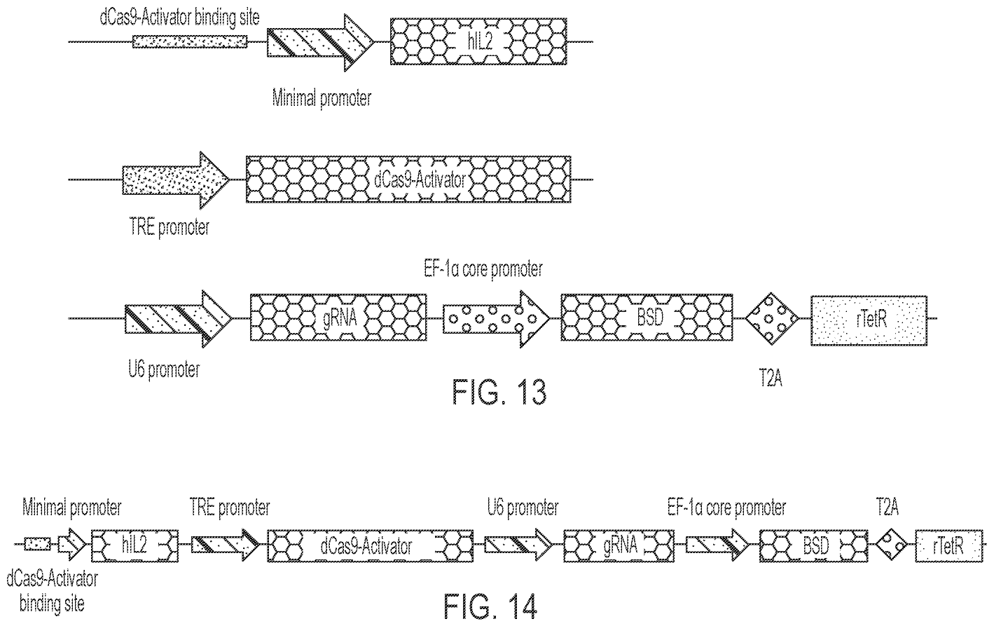

To generate these engineered cells, the cassettes of the minimal promoter-therapeutic gene, inducible promoter-dCas9-activator gene, and U6-gRNA are integrated into the genome of ARPE-19 cells by lentiviral transduction (FIG. 13). In addition, the constructs carrying both the minimal promoter-therapeutic gene cassette and the inducible promoter-dCas9-activator gene cassette will carry antibiotic resistance gene cassettes. This allows the transduced cells to be selected with the corresponding antibiotics. In addition, in order to reduce the toxicity of antibiotic resistance, the antibiotic resistance gene cassettes are flanked by a LoxP site, allowing for the antibiotic resistance gene cassettes to be removed by introducing Cre enzymes into the cells.

As an alternative method to generate these engineered cells, the various component cassettes are integrated into the AAV safe harbor by the AAVS1-targeting HR donor vectors (FIG. 14).

TABLE 1
Exemplary Sequences.

| | |
|---|---|
| Name | Sequence |

Promoters

| | |
|---|---|
| EF-1α | GGGCAGAGCGCACATCGCCCACAGTCCCCGAGAAGTTGGGGGGAGGGG |
| | TCGGCAATTGATCCGGTGCCTAGAGAAGGTGGCGCGGGGTAAACTGGG |
| | AAAGTGATGTCGTGTACTGGCTCCGCCTTTTTCCCGAGGGTGGGGGAG |
| | AACCGTATATAAGTGCAGTAGTCGCCGTGAACGTTCTTTTTCGCAACG |
| | GGTTTGCCGCCAGAACACAG (SEQ ID NO: 1) |
| CMV | GACATTGATTATTGACTAGTTATTAATAGTAATCAATTACGGGGTCAT |
| | TAGTTCATAGCCCATATATGGAGTTCCGCGTTACATAACTTACGGTAA |
| | ATGGCCCGCCTGGCTGACCGCCCAACGACCCCCGCCCATTGACGTCAA |
| | TAATGAGGTATGTTCCCATAGTAACGCCAATAGGGAGTTTCCATTGAG |
| | GTCAATGGGTGGAGTATTTACGGTAAACTGCCCACTTGGCAGTACATC |
| | AAGTGTATCATATGCCAAGTACGCCCCCTATTGACGTCAATGACGGTA |
| | AATGGCCCGCCTGGCATTATGCCCAGTACATGACCTTATGGGACTTTC |
| | CTACTTGGCAGTAGATCTACGTATTAGTCATCGCTATTACCATGGTGA |
| | TGCGGTTTTGGCAGTACATCAATGGGCGTGGATAGCGGTTTGACTCAC |
| | GGGGATTTCCAAGTCTCCACCCCATTGACGTCAATGGGAGTTTGTTTT |
| | GGCACCAAAATCAACGGGACTTTCCAAAATGTCGTAACAACTCCGCCC |
| | CATTGACGCAAATGGGCGGTAGGCGTGTACGGTGGGAGGTCTATATAA |
| | GCAGAGCT (SEQ ID NO: 2) |
| Ubc | GGCCTCCGCGCCGGGTTTTGGCGCCTCCCGCGGGCGCCCCCCTCCTCA |
| | CGGCGAGCGCTGCCACGTCAGACGAAGGGCGCAGGAGCGTTCCTGATC |
| | CTTCCGCCCGGACGCTCAGGACAGCGGCCCGCTGCTCATAAGACTCGG |
| | CCTTAGAACCCCAGTATCAGCAGAAGGACATTTTAGGACGGGACTTGG |
| | GTGACTCTAGGGCACTGGTTTTCTTTCCAGAGAGCGGAACAGGCGAGG |
| | AAAAGTAGTCCCTTCTCGGCGATTCTGCGGAGGGATCTCCGTGGGGCG |
| | GTGAACGCCGATGATTATATAAGGACGCGCCGGGTGTGGCACAGCTAG |
| | TTCCGTCGCAGCCGGGATTTGGGTCGCGGTTCTTGTTTGTGGATCGCT |
| | GTGATCGTCACTTGGTGAGTTGCGGGCTGCTGGGCTGGCCGGGGCTTT |
| | CGTGGCCGCCGGGCCGCTCGGTGGGACGGAAGCGTGTGGAGAGACCGC |
| | CAAGGGCTGTAGTCTGGGTCCGCGAGCAAGGTTGCCCTGAACTGGGGG |
| | TTGGGGGGAGCGCACAAAATGGCGGCTGTTCCCGAGTCTTGAATGGAA |
| | GACGCTTGTAAGGCGGGCTGTGAGGTCGTTGAAACAAGGTGGGGGGCA |
| | TGGTGGGCGGCAAGAACCCAAGGTCTTGAGGCCTTCGCTAATGCGGGA |
| | AAGCTCTTATTCGGGTGAGATGGGCTGGGGCACCATCTGGGGACCCTG |
| | ACGTGAAGTTTGTCACTGACTGGAGAACTCGGGTTTGTCGTCTGGTTG |
| | CGGGGGCGGCAGTTATGCGGTGCCGTTGGGCAGTGCACCCGTACCTTT |
| | GGGAGCGCGCGCCTCGTCGTGTCGTGACGTCACCCGTTCTGTTGGCTT |
| | ATAATGCAGGGTGGGGCCACCTGCCGGTAGGTGTGCGGTAGGCTTTTC |
| | TCCGTCGCAGGACGCAGGGTTCGGGCCTAGGGTAGGCTCTCCTGAATC |
| | GACAGGCGCCGGACCTCTGGTGAGGGGAGGGATAAGTGAGGCGTCAGT |
| | TTCTTTGGTCGGTTTTATGTACCTATCTTCTTAAGTAGCTGAAGCTCC |
| | GGTTTTGAACTATGCGCTCGGGGTTGGCGAGTGTGTTTTGTGAAGTTT |
| | TTTAGGCACCTTTTGAAATGTAATCATTTGGGTCAATATGTAATTTTC |
| | AGTGTTAGACTAGTAAATTGTCCGCTAAATTCTGGCCGTTTTTGGCTT |
| | TTTTGTTAGAC (SEQ ID NO: 3) |
| hPGK | GGGGTTGGGGTTGCGCCTTTTCCAAGGCAGCCCTGGGTTTGCGCAGGG |
| | ACGCGGCTGCTCTGGGCGTGGTTCCGGGAAACGCAGCGGCGCCGACCC |
| | TGGGTCTCGCACATTCTTCACGTCCGTTCGCAGCGTCACCCGGATCTT |
| | CGCCGCTACCCTTGTGGGCCCCCCGGCGACGCTTCCTGCTCCGCCCCT |
| | AAGTCGGGAAGGTTCCTTGCGGTTCGCGGCGTGCCGGACGTGACAAAC |
| | GGAAGCCGCACGTCTCACTAGTACCCTCGCAGACGGACAGCGCCAGGG |
| | AGCAATGGCAGCGCGCCGACCGCGATGGGCTGTGGCCAATAGCGGCTG |
| | CTCAGCAGGGCGCGCCGAGAGCAGCGGCCGGGAAGGGGCGGTGCGGGA |
| | GGCGGGGTGTGGGGCGGTAGTGTGGGCCCTGTTCCTGCCCGCGCGGTG |
| | TTCCGCATTCTGCAAGCCTCCGGAGCGCACGTCGGCAGTCGGCTCCCT |
| | CGTTGACCGAATCACCGACCTCTCTCCCCAG (SEQ ID NO: 4) |
| VMD2 | GAATTCTGTCATTTTACTAGGGTGATGAAATTCCCAAGCAACACCATC |
| | CTTTTCAGATAAGGGCACTGAGGCTGAGAGAGGAGCTGAAACCTACCC |
| | GGGGTCACCACACACAGGTGGCAAGGCTGGGACCAGAAACCAGGACTG |
| | TTGACTGCAGCCCGGTATTCATTCTTTCCATAGCCCACAGGGCTGTCA |
| | AAGACCCCAGGGCCTAGTCAGAGGCTCCTCCTTCCTGGAGAGTTCCTG |
| | GCACAGAAGTTGAAGCTCAGCACAGCCCCCTAACCCCCAACTCTCTCT |
| | GCAAGGCCTCAGGGGTCAGAACACTGGTGGAGCAGATCCTTTAGCCTC |
| | TGGATTTTAGGGCCATGGTAGAGGGGGTGTTGCCCTAAATTCCAGCCC |
| | TGGTCTCAGCCCAACACCCTCCAAGAAGAAATTAGAGGGGCCATGGCC |
| | AGGCTGTGCTAGCCGTTGCTTCTGAGCAGATTACAAGAAGGGACTAAG |
| | ACAAGGACTCCTTTGTGGAGGTCCTGGCTTAGGGAGTCAAGTGACGGC |
| | GGCTCAGCACTCACGTGGGCAGTGCCAGCCTCTAAGAGTGGGCAGGGG |
| | CACTGGCCACAGAGTCCCAGGGAGTCCCACCAGCCTAGTCGCCAGACC |
| | (SEQ ID NO: 5) |
| CAG | CACATTGATTATTGACTAGTTATTAATAGTAATCAATTACGGGGTCAT |
| | TAGTTCATAGCCCATATATGGAGTTCCGCGTTACATAACTTACGGTAA |
| | ATGGCCCGCCTGGCTGACCGCCCAACGACCCCCGCCCATTGACGTCAA |
| | TAATGACGTATGTTCCCATAGTAACGCCAATAGGGACTTTCCATTGAC |
| | GTCAATGGGTGGAGTATTTACGGTAAACTGCCCACTTGGCAGTACATC |
| | AAGTGTATCATATGCCAAGTACGCCCCCTATTGACGTCAATGACGGTA |
| | AATGGCCCGCCTGGCATTATGCCCAGTACATGACCTTATGGGACTTTC |
| | CTACTTGGCAGTAGATCTACGTATTAGTCATCGCTATTACCATGGTCG |
| | AGGTGAGCCCCACGTTCTGCTTCACTCTCCCCATCTCCCCCCCCTCCC |
| | CACCCCCAATTTTGTATTTATTTATTTTTTAATTATTTTGTGCAGCGA |
| | TGGGGGCGGGGGGGGGGGGGGGGCGCGCGCCAGGCGGGGCGGGGCGGG |
| | GCGAGGGGCGGGGCGGGGCGAGGCGGAGAGGTGCGGCGGCAGCCAATC |
| | AGAGCGGCGCGCTCCGAAAGTTTCCTTTTATGGCGAGGCGGCGGCGGC |
| | GGCGGCCCTATAAAAAGCGAAGCGCGCGGCGGGCGGGGAGTCGCTGCG |
| | ACGCTGCCTTCGCCCCGTGCCCCGCTCCGCCGCCGCCTCGCGCCGCCC |
| | GCCCCGGCTCTGACTGACCGCGTTACTCCCACAGGTGAGCGGGCGGGA |
| | CGGCCCTTCTCCTCCGGGCTGTAATTAGCGCTTGGTTTAATGACGGCT |
| | TGTTTCTTTTCTGTGGCTGCGTGAAAGCCTTGAGGGGCTCCGGGAGGG |
| | CCCTTTGTGCGGGGGGAGCGGCTCGGGGGGTGCGTGCGTGTGTGTGTG |
| | CGTGGGGAGCGCCGCGTGCGGCTCCGCGCTGCCCGGCGGCTGTGAGCG |
| | CTGCGGGCGCGGCGCGGGGCTTTGTGCGCTCCGCAGTGTGCGCGAGGG |
| | GAGCGCGGCCGGGGGCGGTGCCCCGCGGTGCGGGGGGGGCTGCGAGGG |
| | GAACAAAGGCTGCGTGCGGGGTGTGTGCGTGGGGGGGTGAGCAGGGGG |
| | TGTGGGCGCGTCGGTCGGGCTGCAACCCCCCCTGCACCCCCCTCCCCG |
| | AGTTGCTGAGCACGGCCCGGCTTCGGGTGCGGGGCTCCGTACGGGGCG |
| | TGGCGCGGGGCTCGCCGTGCCGGGCGGGGGGTGGCGGCAGGTGGGGGT |
| | GCCGGGCGGGGCGGGGCCGCCTCGGGCCGGGGAGGGCTCGGGGGAGGG |
| | GCGCGGCGGCCCCCGGAGCGCCGGCGGCTGTCGAGGCGCGGCGAGCCG |
| | CAGCCATTGCCTTTTATGGTAATCGTGCGAGAGGGCGCAGGGACTTCC |
| | TTTGTCCCAAATCTGTGCGGAGCCGAAATCTGGGAGGCGCCGCCGCAC |
| | CCCCTCTAGCGGGCGCGGGGCGAAGCGGTGCGGCGCCGGCAGGAAGGA |
| | AATGGGCGGGGAGGGCCTTCGTGCGTCGCCGCGCCGCCGTCCCCTTCT |
| | CCCTCTCCAGCCTCGGGGCTGTCCGCGGGGGGACGGCTGCCTTCGGGG |
| | GGGACGGGGCAGGGCGGGGTTCGGCTTCTGGCGTGTGACCGGCGGCTC |
| | TAGAGCCTCTGCTAACCATGTTCATGCCTTCTTCTTTTTCCTACAG |
| | (SEQ ID NO: 6) |
| TRE | GAGTTTACTCCCTATCAGTGATAGAGAACGTATGTCGAGTTTACTCCC |
| | TATCAGTGATAGAGAACGATGTCGAGTTTACTCCCTATCAGTGATAGA |
| | GAACGTATGTCGAGTTTACTCCCTATCAGTGATAGAGAACGTATGTCG |
| | AGTTTACTCCCTATCAGTGATAGAGAACGTATGTCGAGTTTATCCCTA |
| | TCAGTGATAGAGAACGTATGTCGAGTTTACTCCCTATCAGTGATAGAG |
| | AACGTATGTCGAGGTAGGCGTGTACGGTGGGAGGCCTATATAAGCAGA |
| | GCTCGTTTAGTGAACCGTCAGATCGCC (SEQ ID NO: 7) |
| hU6 | CGAGGGCCTATTTCCCATGATTCCTTCATATTTGCATATACGATACAA |
| | GGCTGTTAGAGAGATAATTGGAATTAATTTGACTGTAAACACAAAGAT |
| | ATTAGTACAAAATACGTGACGTAGAAAGTAATAATTTCTTGGGTAGTT |
| | TGCAGTTTTAAAATTATGTTTTAAAATGGACTATCATATGCTTATCGT |
| | AACTTGAAAGTATTTCGATTTCTTGGCTTTATATATCTTGTGGAAAGG |
| | ACGAAACACC (SEQ ID NO: 8) |

Activators

| | |
|---|---|
| TET1 catalytic | LPTCSCLDRVIQKDKGPYYTHLGAGPSVAAVREIMENRYGQKGNAIRI |
| domain | EIVVYTGKEGKSSHGCPIAKWVLRRSSDEEKVLCLVRQRTGHHCPTAV |
| | MVVLIMVWDGIPLPMADRLYTELTENLKSYNGHPTDRRCTLNENRTCT |
| | CQGIDPETCGASFSFGCSWSMYFNGCKFGRSPSPRRFRIDPSSPLHEK |
| | NLEDNLQSLATRLAPIYKQYAPVAYQNQVEYENVARECRLGSKEGRPF |
| | SGVTACLDFCAHPHRDIHNMNNGSTVVCTLTREDNRSLGVIPQDEQLH |
| | VLPLYKLSDTDEFGSKEGMEAKIKSGAIEVLAPRRKKRTCFTQPVPRS |
| | GKKRAAMMTEVLAHKIRAVEKKPIPRIKRKNNSTTTNNSKPSSLPTLG |
| | SNTETVQPEVKSETEPHFILKSSDNTKTYSLMPSAPHPVKEASPGFSW |
| | SPKTASATPAPLKNDATASCGFSERSSTPHCTMPSGRLSGANAAAADG |
| | PGISQLGEVAPLPTLSAPVMEPLINSEPSTGVTEPLTPHQPNHQPSFL |
| | TSPQDLASSPMEEDEQHSEADEPPSDEPLSDDPLSPAEEKLPHIDEYW |
| | SDSEHIFLDANIGGVAIAPAHGSVLIECARRELHATTPVEHPNRNHPT |
| | RLSLVFYQHKNLNKPQHGEELNKIKFEAKEAKNKKMKASEQKDQAANE |
| | GPEQSSEVNELNQIPSHKALTLTHDNVVTVSPYALTHVAGPYNHWV |
| | (SEQ ID NO: 9) |
| P300 core | IFKPEELRQALMPTLEALYRQDPESLPFRQPVDPQLLGIPDYFDIVKS |
| | PMDLSTIKRKLDTGQYQEPWQYVDDIWLMFNNAWLYNRKTSRVYKYCS |
| | KLSEVFEQEIDPVMQSLGYCCGRKLEFSPQTLCCYGKQLCTIPRDATY |
| | YSYQNRYHFCEKCFNEIQGESVSLGDDPSQPQTTINKEQFSKRKNDTL |
| | DPELFVECTECGRKMHQICVLHHEIIWPAGFVCDGCLKKSARTRKENK |
| | FSAKRLPSTRLGTFLENRVNDFLRRQNHPESGEVTVRVVHASDKTVEV |
| | KPGMKARFVDSGEMAESFPYRTKALFAFEEIDGVDLCFFGMHVQEYGS |
| | DCPPPNQRRVYISYLDSVHFFRPKCLRTAVYHEILIGYLEYVKKLGYT |
| | TGHIWACPPSEGDDYIFHCHPPDQKIPKPKRLQEWYKKMLDKAVSERI |
| | VHDYKDIFKQATEDRLTSAKELPYFEGDFWPNVLEESIKELEQEEEER |
| | KREENTSNESTDVTKGDSKNAKKKNNKKTSKNKSSLSRGNKKKPGMPN |
| | VSNDLSQKLYATMEKHKEVFFVIRLIAGPAANSLPPIVDPDPLIPCDL |
| | MDGRDAFLTLARDKHLEFSSLRRAQWSTMCMLVELHTQSQD (SEQ |
| | ID NO: 10) |
| VPR | DALDDFDLDMLGSDALDDFDLDMLGSDALDDFDLDMLGSDALDDFDLD |
| | MLINSRSSGSPKKKRKVGSQYLPDTDDRHRIEEKRKRTYETFKSIMKK |
| | SPFSGPTDPRPPPRRIAVPSRSSASVPKPAPQPYPFTSSLSTINYDEF |
| | PTMVFPSGQISQASALAPAPPQVLPQAPAPAPAPAMVSALAQAPAPVP |
| | VLAPGPPQAVAPPAPKPTQAGEGTLSEALLQLQFDDEDLGALLGNSTD |
| | PAVFTDLASVDNSEFQQLLNQGIPVAPHTTEPMLMEYPEAITRLVTGA |
| | QRPPDPAPAPLGAPGLPNGLLSGDEDFSSIADMDFSALLGSGSGSRDS |
| | REGMFLPKPEAGSAISDVFEGREVCQPKRIRPFHPPGSPWANRPLPAS |
| | LAPTPTGPVHEPVGSLTPAPVPQPLDPAPAVTPEASHLLEDPDEETSQ |
| | AVKALREMADTVIPQKEEAAICGQMDLSHPPPRGHLDELTTTLESMTE |
| | DLNLDSPLTPELNEILDTFLNDECLLHAMHISTGLSIFDTSLF (SEQ |
| | ID NO: 11) |
| rTetR | MSRLDKSKVINGALELLNGVGIEGLTTRKLAQKLGVEQPTLYWHVKNK |
| | RALLDALPIEMLDRHHTHFCPLEGESWQDFLRNNAKSYRCALLSHRDG |
| | AKVHLGTRPTEKQYETLENQLAFLCQQGFSLENALYALSAVGHFTLGC |
| | VLEEQEHQVAKEERETPTTDSMPPLLRQAIELFDRQGAEPAFLFGLEL |
| | IICGLEKQLKCESGGPTDALDDFDLDMLPADALDDFDLDMLPADALDD |
| | FDLDMLPG (SEQ ID NO: 12) |

dCas9 proteins

| | |
|---|---|
| Streptococcus | MDKKYSIGLAIGTNSVGWAVITDEYKVPSKKFKVLGNTDRHSIKKNLI |
| pyogenes Cas9 | GALLFDSGETAEATRLKRTARRRYTRRKNRICYLQEIFSNEMAKVDDS |
| (D10A, H840A) | FFHRLEESFLVEEDKKHERHPIFGNIVDEVAYHEKYPTIYHLRKKLVD |
| | STDKADLRLIYLALAHMIKFRGHFLIEGDLNPDNSDVDKLFIQLVQTY |
| | NQLFEENPINASGVDAKAILSARLSKSRRLENLIAQLPGEKKNGLFGN |
| | LIALSLGLTPNFKSNFDLAEDAKLQLSKDTYDDDLDNLLAQIGDQYAD |
| | LFLAAKNLSDAILLSDILRVNTEITKAPLSASMIKRYDEHHQDLTLLK |
| | ALVRQQLPEKYKEIFFDQSKNGYAGYIDGGASQEEFYKFIKPILEKMD |
| | GTEELLVKLNREDLLRKQRTFDNGSIPHQIHLGELHAILRRQEDFYPF |
| | LKDNREKIEKILTFRIPYYVGPLARGNSRFAWMTRKSEETITPWNFEE |
| | VVDKGASAQSFIERMTNFDKNLPNEKVLPKHSLLYEYFTVYNELTKVK |
| | YVTEGMRKPAFLSGEQKKAIVDLLFKTNRKVTVKQLKEDYFKKIECFD |
| | SVEISGVEDRFNASLGTYHDLLKIIKDKDFLDNEENEDILEDIVLTLT |
| | LFEDREMIEERLKTYAHLFDDKVMKQLKRRRYTGWGRLSRKLINGIRD |
| | KQSGKTILDFLKSDGFANRNFMQLIHDDSLTFKEDIQKAQVSGQGDSL |
| | HEHIANLAGSPAIKKGILQTVKVVDELVKVMGRHKPENIVIEMARENQ |
| | TTQKGQKNSRERMKRIEEGIKELGSQILKEHPVENTQLQNEKLYLYYL |
| | QNGRDMYVDQELDINRLSDYDVDAIVPQSFLKDDSIDNKVLTRSDKNR |
| | GKSDNVPSEEVVKKMKNYWRQLLNAKLITQRKFDNLTKAERGGLSELD |
| | KAGFIKRQLVETRQITKHVAQILDSRMNTKYDENDKLIREVKVITLKS |
| | KLVSDFRKDFQFYKVREINNYHHAHDAYLNAVVGTALIKKYPKLESEF |
| | VYGDYKVYDVRKMIAKSEQEIGKATAKYFFYSNIMNFFKTEITLANGE |
| | IRKRPLIETNGETGEIVWDKGRDFATVRKVLSMPQVNIVKKTEVQTGG |
| | FSKESILPKRNSDKLIARKKDWDPKKYGGFDSPTVAYSVLVVAKVEKG |
| | KSKKLKSVKELLGITIMERSSFEKNPIDFLEAKGYKEVKKDLIIKLPK |
| | YSLFELENGRKRMASAGELQKGNELALPSKYVNFLYLASHYEKLKGSP |
| | EDNEQKQLFVEQHKHYLDEIIEQISEESKRVILADANLDKVLSAYNKH |
| | RDKPIREQAENIIHLFTLTNLGAPAAFKYFDTTIDRKRYTSTKEVLDA |
| | TLIHQSITGLYETRIDLSQLGGD (SEQ ID NO: 13) |
| Staphylococcus | MKRNYILGLAIGITSVGYGIIDYETRDVIDAGVRLFKEANVENNEGRR |
| aureus Cas9 | SKRGARRLKRRRRHRIQRVKKLLFDYNLLTDHSELSGINPYEARVKGL |
| (D10A and | SQKLSEEEFSAALLHLAKRRGVHNVNEVEEDTGNELSTKEQISRNSKA |
| N580A) | LEEKYVAELQLERLKKDGEVRGSINRFKTSDYVKEAKQLLKVQKAYHQ |
| | LDQSFIDTYIDLLETRRTYYEGPGEGSPFGWKDIKEWYEMLMGHCTYF |
| | PEELRSVKYAYNADLYNALNDLNNLVITRDENEKLEYYEKFQIIENVF |
| | KQKKKPTLKQIAKEILVNEEDIKGYRVTSTGKPEFTNLKVYHDIKDIT |
| | ARKEIIENAELLDQIAKILTIYQSSEDIQEELTNLNSELTQEEIEQIS |
| | NLKGYTGTHNLSLKAINLILDELWHTNDNQIAIFNRLKLVPKKVDLSQ |
| | QKEIPTTLVDDFILSPVVKRSFIQSIKVINAIIKKYGLPNDIIIELAR |
| | EKNSKDAQKMINEMQKRNRQTNERIEEIIRTTGKENAKYLIEKIKLHD |
| | MQEGKCLYSLEAIPLEDLLNNPFNYEVDHIIPRSVSFDNSFNNKVLVK |
| | QEEASKKGNRTPFQYLSSSDSKISYETFKKHILNLAKGKGRISKTKKE |
| | YLLEERDINRFSVQKDFINRNLVDTRYATRGLMNLLRSYFRVNNLDVK |
| | VKSINGGFTSFLRRKWKFKKERNKGYKHHAEDALIIANADFIFKEWKK |
| | LDKAKKVMENQMFEEKQAESMPEIETEQEYKEIFITPHQIKHIKDFKD |
| | YKYSHRVDKKPNRELINDTLYSTRKDDKGNTLIVNNLNGLYDKDNDKL |
| | KKLINKSPEKLLMYHHDPQTYQKLKLIMEQYGDEKNPLYKYYEETGNY |
| | LTKYSKKDNGPVIKKIKYYGNKLNAHLDITDDYPNSRNKVVKLSLKPY |
| | RFDVYLDNGVYKFVTVKNLDVIKKENYYEVNSKCYEEAKKLKKISNQA |
| | EFIASFYNNDLIKINGELYRVIGVNNDLLNRIEVNMIDITYREYLENM |
| | NDKRPPRIIKTIASKTQSIKKYSTDILGNLYEVKSKKHPQIIKKG |
| | (SEQ ID NO: 14) |
| Lachnospiraceae | MSKLEKFTNCYSLSKTLRFKAIPVGKTQENIDNKRLLVEDEKRAEDYK |
| bacterium Cpf1 | GVKKLLDRYYLSFINDVLHSIKLKNLNNYISLFRKKTRTEKENKELEN |
| (D832A) | LEINLRKEIAKAFKGNEGYKSLFKKDIIETILPEFLDDKDEIALVNSF |
| | NGFTTAFTGFFDNRENMFSEEAKSTSIAFRCINENLTRYISNMDIFEK |
| | VDAIFDKHEVQEIKEKILNSDYDVEDFFEGEFFNFVLTQEGIDVYNAI |
| | IGGFVTESGEKIKGLNEYINLYNQKTKQKLPKFKPLYKQVLSDRESLS |
| | FYGEGYTSDEEVLEVFRNTLNKNSEIFSSIKKLEKLFKNFDEYSSAGI |
| | FVKNGPAISTISKDIFGEWNVIRDKWNAEYDDIHLKKKAVVTEKYEDD |
| | RRKSFKKIGSFSLEQLQEYADADLSVVEKLKEIIIQKVDEIYKVYGSS |
| | EKLFDADFVLEKSLKKNDAVVAIMKDLLDSVKSFENYIKAFFGEGKET |
| | NRDESFYGDFVLAYDILLKVDHIYDAIRNYVTQKPYSKDKFKLYFQNP |
| | QFMGGWDKDKETDYRATILRYGSKYYLAIMDKKYAKCLQKIDKDDVNG |
| | NYEKINYKLLPGPNKMLPKVFFSKKWMAYYNPSEDIQKIYKNGTFKKG |
| | DMFNLNDCHKLIDFFKDSISRYPKWSNAYDFNFSETEKYKDIAGFYRE |
| | VEEQGYKVSFESASKKEVDKLVEEGKLYMFQIYNKDFSDKSHGTPNLH |
| | TMYFKLLFDENNHGQIRLSGGAELFMRRASLKKEELVVHPANSPIANK |
| | NPDNPKKTTTLSYDVYKDKRFSEDQYELHIPIAINKCPKNIFKINTEV |
| | RVLLKHDDNPYVIGIARGERNLLYIVVVDGKGNIVEQYSLNEIINNFN |
| | GIRIKTDYHSLLDKKEKERFEARQNWTSIENIKELKAGYISQVVHKIC |
| | ELVEKYDAVIALEDLNSGFKNSRVKVEKQVYQKFEKMLIDKLNYMVDK |
| | KSNPCATGGALKGYQITNKFESFKSMSTQNGFIFYIPAWLTSKIDPST |
| | GFVNLLKTKYTSIADSKKFISSFDRIMYVPEEDLFEFALDYKNFSRTD |
| | ADYIKKWKLYSYGNRIRIFRNPKKNNVFDWEEVCLTSAYKELFNKYGI |
| | NYQQGDIRALLCEQSDKAFYSSFMALMSLMLQMRNSITGRTDVDFLIS |
| | PVKNSDGIFYDSRNYEAQENAILPKNADANGAYNIARKVLWAIGQFKK |
| | AEDEKLDKVKIAISNKEWLEYAQTSVKH (SEQ ID NO: 15) |

GRNAs

| | |
|---|---|
| Streptococcus | protospacer- |
| pyogenes Cas9 | GTTTTAGAGCTAGAAATAGCAAGTTAAAATAAGGCTAGTCCGTTATCA |
| (D10A, H840A) | ACTTGAAAAAGTGGCACCGAGTCGGTGCTTTTTTT (SEQ ID NO: |
| | 16) |
| Staphylococcus | protospacer- |
| aureus Cas9 | GTTTTAGTACTCTGGAAACAGAATCTACTAAAACAAGGCAAAATGCCG |
| (D10A and | TGTTTATCTCGTCAACTTGTTGGCGAGA (SEQ ID NO: 17) |
| N580A) | |
| Lachnospiraceae | AAACACCGAATTTCTACTAAGTGT-protospacer (SEQ ID NO: |
| bacterium Cpf1 | 18) |
| (D832A) | |

Skipping peptides and others

| | |
|---|---|
| T2A | (GSG)EGRGSLLTCGDVEENPGP (SEQ ID NO: 19) |
| P2A | (GSG)ATNFSLLKQAGDVEENPGP (SEQ ID NO: 20) |
| E2A | (GSG)QCTNYALLKLAGDVESNPGP (SEQ ID NO: 21) |
| F2A | (GSG)VKQTLNFDLLKLAGDVESNPGP (SEQ ID NO: 22) |

loxP

| | |
|---|---|
| loxP | ATAACTTCGTATAGCATACATTATACGAAGTTAT (SEQ ID NO: |
| | 23) |

Cell Export Peptides

| | |
|---|---|
| Human OSM | MGVLLTQRTLLSLVLALLFPSMASM (SEQ ID NO: 24) |
| VSV-G | MKCLLYLAFLFIGVNC (SEQ ID NO: 25) |
| Mouse Ig Kappa | METDTLLLWVLLLWVPGSTGD (SEQ ID NO: 26) |
| Human IgG2 H | MGWSCIILFLVATATGVHS (SEQ ID NO: 27) |
| BM40 | MRAWIFFLLCLAGRALA (SEQ ID NO: 28) |
| Secrecon | MWWRLWWLLLLLLLLWPMVWA (SEQ ID NO: 29) |
| Human IgK VIII | MDMRVPAQLLGLLLLWLRGARC (SEQ ID NO: 30) |
| CD33 | MPLLLLLPLLWAGALA (SEQ ID NO: 31) |
| tPA | MDAMKRGLCCVLLLCGAVFVSPS (SEQ ID NO: 32) |
| Human | MAFLWLLSCWALLGTTFG (SEQ ID NO: 33) |
| Chymotrypsinogen | |
| Human | MNLLLILTFVAAAVA (SEQ ID NO: 34) |
| trypsinogen-2 | |
| Human IL-2 | MYRMQLLSCIALSLALVTNS (SEQ ID NO: 35) |
| Gaussia luc | MGVKVLFALICIAVAEA (SEQ ID NO: 36) |
| Albumin(HSA) | MKWVTFISLLFSSAYS (SEQ ID NO: 37) |
| Influenza | MKTIIALSYIFCLVLG (SEQ ID NO: 38) |
| Haemagglutinin | |
| Human insulin | MALWMRLLPLLALLALWGPDPAAA (SEQ ID NO: 39) |
| Silkworm | MKPIFLVLLVVTSAYA (SEQ ID NO: 40) |
| Fibroin LC | |

Antibiotic Resistance Genes

| | |
|---|---|
| PuroR | MTEYKPTVRLATRDDVPRAVRTLAAAFADYPATRHTVDPDRHIERVTE |
| | LQELFLTRVGLDIGKVWVADDGAAVAVWTTPESVEAGAVFAEIGPRMA |
| | ELSGSRLAAQQQMEGLLAPHRPKEPAWFLATVGVSPDHQGKGLGSAVV |
| | LPGVEAAERAGVPAFLETSAPRNLPFYERLGFTVTADVEVPEGPRTWC |
| | MTRKPGA (SEQ ID NO: 41) |
| NeoR | MIEQDGLHAGSPAAWVERLFGYDWAQQTIGCSDAAVFRLSAQGRPVLF |
| | VKTDLSGALNELQDEAARLSWLATTGVPCAAVLDVVTEAGRDWLLLGE |
| | VPGQDLLSSHLAPAEKVSIMADAMRRLHTLDPATCPFDHQAKHRIERA |
| | RTRMEAGLVDQDDLDEEHQGLAPAELFARLKASMPDGEDLVVTHGDAC |
| | LPNIMVENGRFSGFIDCGRLGVADRYQDIALATRDIAEELGGEWADRF |
| | LVLYGIAAPDSQRIAFYRLLDEFF (SEQ ID NO: 42) |
| BSD | MAKPLSQEESTLIERATATINSIPISEDYSVASAALSSDGRIFTGVNV |
| | YHFTGGPCAELVVLGTAAAAAAGNLTCIVAIGNENRGILSPCGRCRQV |
| | LLDLHPGIKAIVKDSDGQPTAVGIRELLPSGYVWEG (SEQ ID NO: |
| | 43) |

Therapeutic Proteins

| | |
|---|---|
| VEGF Alpha | ATGACGGACAGACAGACAGACACCGCCCCCAGCCCCAGCGCCCACCTC |
| | CTCGCCGGCGGGCAGCCGACGGTGGACGCGGCGGCGAGCCGCGAGCAG |
| | GAGCCGAAGCCCGCGCCCGGAGGCGGGGTGGAGGGGGTCGGGGCTCGC |
| | GGGATTGCACGGAAACTTTTCGTCCAACTTCTGGGCTCTTCTCTCTCC |
| | GGAGTAGCCGTGGTCTGCGCCGCAGGAGGCAAACCGATCGGAGCTGGG |
| | AGAAGTGCTAGCTCGGGCCTGGAGAAGCCGGGGCCCGAGAAGAGAGGG |
| | GAGAAAGAGAAGGAAGAGGAGAGGGGGCCGCAGTGGGCGCTCGGCTCT |
| | CGGGAGCCGGGCTCATGGACGGGTGAGGCGGCGGTGTGCGCAGACAGT |
| | GCTCCAGCCGCGCGCGCGCCCCAGGCCCCGGCCCGGGCCTCGGTTCCA |
| | GAAGGGAGAGGAGCCCGCCAAGGCGCGCAAGAGAGCGGGCTGCCTCGC |
| | AGTCCGAGCCGGAGAGGGAGCGCGAGCCGCGCCGGCCCCGGACGGGCC |
| | TCTGAAACCATGAACTTTCTGCTCTCTTGGGTGCACTGGACCCTGGCT |
| | TTACTGCTGTACCTCCACCATGCCAAGTGGTCCCAGGCTGCACCCACG |
| | ACAGAAGGGGAGCAGAAAGCCCATGAAGTGGTGAAGTTCATGGACGTC |
| | TACCAGCGCAGCTATTGCCGTCCAATTGAGACCCTGGTGGACATCTTC |
| | CAGGAGTACCCCGATGAGATAGAGTATATCTTCAAGCCGTCCTGTGTG |
| | CCCCTAATGCGGTGTGCGGGCTGCTGCAATGATGAAGCCCTGGAGTGC |
| | GTGCCCACGTCGGAGAGCAACGTCACTATGCAGATCATGCGGATCAAA |
| | CCTCACCAAAGCCAGCACATAGGAGAGATGAGCTTCCTGCAGCATAGC |
| | AGATGTGAATGCAGACCAAAGAAAGATAGAACAAAGCCAGAAAAAAAA |
| | TCAGTTCGAGGAAAGGGAAAGGGTCAAAAACGAAAGCGCAAGAAATCC |
| | CGGTTTAAATCCTGGAGCGTTCACTGTGAGCCTTGTTCAGAGCGGAGA |
| | AAGCATTTGTTTGTCCAAGATCCGCAGACGTGTAAATGTTCCTGCAAA |
| | AACACAGACTCGCGTTGCAAGGCGAGGCAGCTTGAGTTAAACGAACGT |
| | ACTTGCAGATGTGACAAGCCAAGGCGGTGA (SEQ ID NO: 44) |
| TGF Beta | ATGCCGCCCTCGGGGCTGCGGCTACTGCCGCTTCTGCTCCCACTCCCG |
| | TGGCTTCTAGTGCTGACGCCCGGGAGGCCAGCCGCGGGACTCTCCACC |
| | TGCAAGACCATCGACATGGAGCTGGTGAAACGGAAGCGCATCGAAGCC |
| | ATCCGTGGCCAGATCCTGTCCAAACTAAGGCTCGCCAGTCCCCCAAGC |
| | CAGGGGGAGGTACCGCCCGGCCCGCTGCCCGAGGCGGTGCTCGCTTTG |
| | TACAACAGCACCCGCGACCGGGTGGCAGGCGAGAGCGCCGACCCAGAG |
| | CCGGAGCCCGAAGCGGACTACTATGCTAAAGAGGTCACCCGCGTGCTA |
| | ATGGTGGACCGCAACAACGCCATCTATGAGAAAACCAAAGACATCTCA |
| | CACAGTATATATATGTTCTTCAATACGTCAGACATTCGGGAAGCAGTG |
| | CCCGAACCCCCATTGCTGTCCCGTGCAGAGCTGCGCTTGCAGAGATTA |
| | AAATCAAGTGTGGAGCAACATGTGGAACTCTACCAGAAATATAGCAAC |
| | AATTCCTGGCGTTACCTTGGTAACCGGCTGCTGACCCCCACTGATACG |
| | CCTGAGTGGCTGTCTTTTGACGTCACTGGAGTTGTACGGCAGTGGCTG |
| | AACCAAGGAGACGGAATACAGGGCTTTCGATTCAGCGCTCACTGCTCT |
| | TGTGACAGCAAAGATAACAAACTCCACGTGGAAATCAACGGGATCAGC |
| | CCCAAACGTCGGGGCGACCTGGGCACCATCCATGACATGAACCGGCCC |
| | TTCCTGCTCCTCATGGCCACCCCCCTGGAAAGGGCCCAGCACCTGCAC |
| | AGCTCACGGCACCGGAGAGCCCTGGATACCAACTATTGCTTCAGCTCC |
| | ACAGAGAAGAACTGCTGTGTGCGGCAGCTGTACATTGACTTTAGGAAG |
| | GACCTGGGTTGGAAGTGGATCCACGAGCCCAAGGGCTACCATGCCAAC |
| | TTCTGTCTGGGACCCTGCCCCTATATTTGGAGCCTGGACACACAGTAC |
| | AGCAAGGTCCTTGCCCTCTACAACCAACACAACCCGGGCGCTTCGGCG |
| | TCACCGTGCTGCGTGCCGCAGGCTTTGGAGCCACTGCCCATCGTCTAC |
| | TACGTGGGTCGCAAGCCCAAGGTGGAGCAGTTGTCCAACATGATTGTG |
| | CGCTCCTGCAAGTGCAGCTGA (SEQ ID NO: 45) |
| IL2 | ATGTACAGGATGCAACTCCTGTCTTGCATTGCACTAAGTCTTGCACTT |
| | GTCACAAACAGTGCACCTACTTCAAGTTCTACAAAGAAAACACAGCTA |
| | CAACTGGAGCATTTACTGCTGGATTTACAGATGATTTTGAATGGAATT |
| | AATAATTACAAGAATCCCAAACTCACCAGGATGCTCACATTTAAGTTT |
| | TAGATGCCCAAGAAGGCCACAGAACTGAAACATCTTCAGTGTCTAGAA |
| | GAAGAACTCAAACCTCTGGAGGAAGTGCTAAATTTAGCTCAAAGCAAA |
| | AACTTTCACTTAAGACCCAGGGACTTAATCAGCAATATCAACGTAATA |
| | GTTCTGGAACTAAAGGGATCTGAAACAACATTCATGTGTGAATATGCT |
| | GATGAGACAGCAACCATTGTAGAATTTCTGAACAGATGGATTACCTTT |
| | TGTCAAAGCATCATCTCAACACTGACTTGA (SEQ ID NO: 46) |
| IL4 | ATGGGTCTCACCTCCCAACTGCTTCCCCCTCTGTTCTTCCTGCTAGCA |
| | TGTGCCGGCAACTTTGTCCACGGACACAAGTGCGATATCACCTTACAG |
| | GAGATCATCAAAACTTTGAACAGCCTCACAGAGCAGAAGACTCTGTGC |
| | ACCGAGTTGACCGTAACAGACATCTTTGCTGCCTCCAAGAACACAACT |
| | GAGAAGGAAACCTTCTGCAGGGCTGCGACTGTGCTCCGGCAGTTCTAC |
| | AGCCACCATGAGAAGGACACTCGCTGCCTGGGTGCGACTGCACAGCAG |
| | TTCCACAGGCACAAGCAGCTGATCCGATTCCTGAAACGGCTCGACAGG |
| | AACCTCTGGGGCCTGGCGGGCTTGAATTCCTGTCCTGTGAAGGAAGCC |
| | AACCAGAGTACGTTGGAAAACTTCTTGGAAAGGCTAAAGACGATCATG |
| | AGAGAGAAATATTCAAAGTGTTCGAGCTGA (SEQ ID NO: 47) |
| IL6 | ATGAACTCCTTCTCCACAAGCGCCTTCGGTCCAGTTGCCTTCTCCCTG |
| | GGGCTGCTCCTGGTGTTGCCTGCTGCCTTCCCTGCCCCAGTACCCCCA |
| | GGAGAAGATTCCAAAGATGTAGCCGCCCCACACAGACAGCCACTCACC |
| | TCTTCAGAACGAATTGACAAACAAATTCGGTAGATCCTCGACGGCATC |
| | TCAGCCCTGAGAAAGGAGACATGTAACAAGAGTAACATGTGTGAAAGC |
| | AGCAAAGAGGCACTGGCAGAAAACAACCTGAACCTTCCAAAGATGGCT |
| | GAAAAAGATGGATGCTTCCAATCTGGATTCAATGAGGAGACTTGCCTG |
| | GTGAAAATCATCACTGGTCTTTTGGAGTTTGAGGTATACCTAGAGTAC |
| | CTCCAGAACAGATTTGAGAGTAGTGAGGAACAAGCCAGAGCTGTGCAG |
| | ATGAGTACAAAAGTCCTGATCCAGTTCCTGCAGAAAAAGGCAAAGAAT |
| | CTAGATGCAATAACCACCCCTGACCCAACCACAAATGCCAGCCTGCTG |
| | ACGAAGCTGCAGGCACAGAACCAGTGGCTGCAGGACATGACAACTCAT |
| | CTCATTCTGCGCAGCTTTAAGGAGTTCCTGCAGTCCAGCCTGAGGGCT |
| | CTTCGGCAAATGTAG (SEQ ID NO: 48) |
| IL7 | ATGTTCCATGTTTCTTTTAGGTATATCTTTGGACTTCCTCCCCTGATC |
| | CTTGTTCTGTTGCCAGTAGCATCATCTGATTGTGATATTGAAGGTAAA |
| | GATGGCAAACAATATGAGAGTGTTCTAATGGTCAGCATCGATCAATTA |
| | TTGGACAGCATGAAAGAAATTGGTAGCAATTGCCTGAATAATGAATTT |
| | AACTTTTTTAAAAGACATATCTGTGATGCTAATAAGGAAGGTATGTTT |
| | TTATTCCGTGCTGCTCGCAAGTTGAGGCAATTTCTTAAAATGAATAGC |
| | ACTGGTGATTTTGATCTCCACTTATTAAAAGTTTCAGAAGGCACAACA |
| | ATACTGTTGAACTGCACTGGCCAGGTTAAAGGAAGAAAACCAGCTGCC |
| | CTGGGTGAAGCCCAACCAACAAAGAGTTTGGAAGAAAATAAATCTTTA |
| | AAGGAACAGAAAAAACTGAATGACTTGTGTTTCCTAAAGAGACTATTA |
| | CAAGAGATAAAAACTTGTTGGAATAAAATTTTGATGGGCACTAAAGAA |
| | CACTGA (SEQ ID NO: 49) |
| IL10 | ATGCACAGCTCAGCACTGCTCTGTTGCCTGGTCCTCCTGACTGGGGTG |
| | AGGGCCAGCCCAGGCCAGGGCACCCAGTCTGAGAACAGCTGCACCCAC |
| | TTCCCAGGCAACCTGCCTAACATGCTTCGAGATCTCCGAGATGCCTTC |
| | AGCAGAGTGAAGACTTTCTTTCAAATGAAGGATCAGCTGGACAACTTG |
| | TTGTTAAAGGAGTCCTTGCTGGAGGACTTTAAGGGTTACCTGGGTTGC |
| | CAAGCCTTGTCTGAGATGATCCAGTTTTACCTGGAGGAGGTGATGCCC |
| | CAAGCTGAGAACCAAGACCCAGACATCAAGGCGCATGTGAACTCCCTG |
| | GGGGAGAACCTGAAGACCCTCAGGCTGAGGCTACGGCGCTGTCATCGA |
| | TTTCTTCCCTGTGAAAACAAGAGCAAGGCCGTGGAGCAGGTGAAGAAT |
| | GCCTTTAATAAGCTCCAAGAGAAAGGCATCTACAAAGCCATGAGTGAG |
| | TTTGACATCTTCATCAACTACATAGAAGCCTAGATGACAATGAAGATA |
| | CGAAACTGA (SEQ ID NO: 50) |
| IL12a | ATGTGGCCCCCTGGGTCAGCCTCCCAGCCACCGCCCTCACCTGCCGCG |
| | GCCACAGGTCTGCATCCAGCGGCTCGCCCTGTGTCCCTGCAGTGCCGG |
| | CTCAGCATGTGTCCAGCGCGCAGCCTCCTCCTTGTGGCTACCCTGGTC |
| | CTCCTGGACCACCTCAGTTTGGCCAGAAACCTCCCCGTGGCCACTCCA |
| | GACCCAGGAATGTTCCCATGCCTTCACCACTCCCAAAACCTGCTGAGG |
| | GCCGTCAGCAACATGCTCCAGAAGGCCAGACAAACTCTAGAATTTTAC |
| | CCTTGCACTTCTGAAGAGATTGATCATGAAGATATCACAAAAGATAAA |
| | ACCAGCACAGTGGAGGCCTGTTTACCATTGGAATTAACCAAGAATGAG |
| | AGTTGCCTAAATTCCAGAGAGACCTCTTTCATAACTAATGGGAGTTGC |
| | CTGGCCTCCAGAAAGACCTCTTTTATGATGGCCCTGTGCCTTAGTAGT |
| | ATTTATGAAGACTTGAAGATGTACCAGGTGGAGTTCAAGACCATGAAT |
| | GCAAAGCTTCTGATGGATCCTAAGAGGCAGATCTTTCTAGATCAAAAC |
| | ATGCTGGCAGTTATTGATGAGCTGATGCAGGCCCTGAATTTCAACAGT |
| | GAGACTGTGCCACAAAAATCCTCCCTTGAAGAACCGGATTTTTATAAA |
| | ACTAAAATCAAGCTCTGCATACTTCTTCATGCTTTCAGAATTCGGGCA |
| | GTGACTATTGATAGAGTGATGAGCTATCTGAATGCTTCCTAA (SEQ |
| | ID NO: 51) |
| IL12b | ATGTGTCACCAGCAGTTGGTCATCTCTTGGTTTTCCCTGGTTTTTCTG |
| | GCATCTCCCCTCGTGGCCATATGGGAACTGAAGAAAGATGTTTATGTC |
| | GTAGAATTGGATTGGTATCCGGATGCCCCTGGAGAAATGGTGGTCCTC |
| | ACCTGTGACACCCCTGAAGAAGATGGTATCACCTGGACCTTGGACCAG |
| | AGCAGTGAGGTCTTAGGCTCTGGCAAAACCCTGACCATCCAAGTCAAA |
| | GAGTTTGGAGATGCTGGCCAGTACACCTGTCACAAAGGAGGCGAGGTT |
| | CTAAGCCATTCGCTCCTGCTGCTTCACAAAAAGGAAGATGGAATTTGG |
| | TCCACTGATATTTTAAAGGACCAGAAAGAACCCAAAAATAAGACCTTT |
| | CTAAGATGCGAGGCCAAGAATTATTCTGGACGTTTCACCTGCTGGTGG |
| | CTGACGACAATCAGTACTGATTTGACATTCAGTGTCAAAAGCAGCAGA |
| | GGCTCTTCTGACCCCCAAGGGGTGACGTGCGGAGCTGCTACACTCTCT |
| | GCAGAGAGAGTCAGAGGGGACAACAAGGAGTATGAGTAGTCAGTGGAG |
| | TGCCAGGAGGACAGTGCCTGCCCAGCTGCTGAGGAGAGTCTGCCCATT |
| | GAGGTCATGGTGGATGCCGTTCACAAGCTCAAGTATGAAAACTACACC |
| | AGCAGCTTCTTCATCAGGGACATCATCAAACCTGACCCACCCAAGAAC |
| | TTGCAGCTGAAGCCATTAAAGAATTCTCGGCAGGTGGAGGTCAGCTGG |
| | GAGTACCCTGACACCTGGAGTACTCCACATTCCTACTTCTCCCTGACA |
| | TTCTGCGTTCAGGTCCAGGGCAAGAGCAAGAGAGAAAAGAAAGATAGA |
| | GTCTTCACGGACAAGACCTCAGCCACGGTCATCTGCCGCAAAAATGCC |
| | AGCATTAGCGTGCGGGCCCAGGACCGCTACTATAGCTCATCTTGGAGC |
| | GAATGGGCATCTGTGCCCTGCAGTTAG (SEQ ID NO: 52) |
| IL15 | ATGAGAATTTCGAAACCACATTTGAGAAGTATTTCCATCCAGTGCTAC |
| | TTGTGTTTACTTCTAAACAGTCATTTTCTAACTGAAGCTGGCATTCAT |
| | GTCTTCATTTTGGGCTGTTTCAGTGCAGGGCTTCCTAAAACAGAAGCC |
| | AACTGGGTGAATGTAATAAGTGATTTGAAAAAAATTGAAGATCTTATT |
| | CAATCTATGCATATTGATGCTAGTTTATATACGGAAAGTGATGTTCAC |
| | CCCAGTTGCAAAGTAACAGCAATGAAGTGCTTTCTCTTGGAGTTACAA |
| | GTTATTTCACTTGAGTCCGGAGATGCAAGTATTCATGATACAGTAGAA |
| | AATCTGATCATCCTAGCAAACAACAGTTTGTCTTCTAATGGGAATGTA |
| | ACAGAATCTGGATGCAAAGAATGTGAGGAACTGGAGGAAAAAAATATT |
| | AAAGAATTTTTGCAGAGTTTTGTACATATTGTCCAAATGTTCATCAAC |
| | ACTTCTTGA (SEQ ID NO: 53) |
| Factor VII | ATGGTCTCCCAGGCCCTCAGGCTCCTCTGCCTTCTGCTTGGGCTTCAG |
| | GGCTGCCTGGCTGCAGGCGGGGTCGCTAAGGCCTCAGGAGGAGAAACA |
| | CGGGACATGCCGTGGAAGCCGGGGCCTCACAGAGTCTTCGTAACCCAG |
| | GAGGAAGCCCACGGCGTCCTGCACCGGCGCCGGCGCGCCAACGCGTTC |
| | CTGGAGGAGCTGCGGCCGGGCTCCCTGGAGAGGGAGTGCAAGGAGGAG |
| | CAGTGCTCCTTCGAGGAGGCCCGGGAGATCTTCAAGGACGCGGAGAGG |
| | ACGAAGCTGTTCTGGATTTCTTACAGTGATGGGGACCAGTGTGCCTCA |
| | AGTCCATGCCAGAATGGGGGCTCCTGCAAGGACCAGCTCCAGTCCTAT |
| | ATCTGCTTCTGCCTCCCTGCCTTCGAGGGCCGGAACTGTGAGACGCAC |
| | AAGGATGACCAGCTGATCTGTGTGAACGAGAACGGCGGCTGTGAGCAG |
| | TACTGCAGTGACCACACGGGCACCAAGCGCTCCTGTCGGTGCCACGAG |
| | GGGTACTCTCTGCTGGCAGACGGGGTGTCCTGCACACCCACAGTTGAA |
| | TATCCATGTGGAAAAATACCTATTCTAGAAAAAAGAAATGCGAGCAAA |
| | CCCCAAGGCCGAATTGTGGGGGGCAAGGTGTGCCCCAAAGGGGAGTGT |
| | CCATGGCAGGTCCTGTTGTTGGTGAATGGAGCTCAGTTGTGTGGGGGG |
| | ACCCTGATCAACACCATCTGGGTGGTCTCCGCGGCCCACTGTTTCGAC |
| | AAAATCAAGAACTGGAGGAACCTGATCGCGGTGCTGGGCGAGCACGAC |
| | CTCAGCGAGCACGACGGGGATGAGCAGAGCCGGCGGGTGGCGCAGGTC |
| | ATCATCCCCAGCACGTACGTCCCGGGCACCACCAACCACGACATCGCG |
| | CTGCTCCGCCTGCACCAGCCCGTGGTCCTCACTGACCATGTGGTGCCC |
| | CTCTGCCTGCCCGAACGGACGTTCTCTGAGAGGACGCTGGCCTTCGTG |
| | CGCTTCTCATTGGTCAGCGGCTGGGGCCAGCTGCTGGACCGTGGCGCC |
| | ACGGCCCTGGAGCTCATGGTCCTCAACGTGCCCCGGCTGATGACCCAG |
| | GACTGCCTGCAGCAGTCACGGAAGGTGGGAGACTCCCCAAATATCACG |
| | GAGTACATGTTCTGTGCCGGCTACTCGGATGGCAGCAAGGACTCCTGC |
| | AAGGGGGACAGTGGAGGCCCACATGCCACCCACTACCGGGGCACGTGG |
| | TACCTGACGGGCATCGTCAGCTGGGGCCAGGGCTGCGCAACCGTGGGC |
| | CACTTTGGGGTGTACACCAGGGTCTCCCAGTACATCGAGTGGCTGCAA |
| | AAGCTCATGCGCTCAGAGCCACGCCCAGGAGTCCTCCTGCGAGCCCCA |
| | TTTCCCTAG (SEQ ID NO: 54) |
| Factor VIII | ATGCAGATAGAATTGTCTACATGTTTTTTCCTTTGCCTGCTCAGATTT |
| | TGCTTCTCCGCAACTCGCCGCTATTACCTGGGTGCTGTTGAGTTGAGC |
| | TGGGATTATATGCAAAGTGATCTCGGAGAACTCCCTGTCGACGCGAGG |
| | TTCCCACCGAGGGTGCCAAAATCTTTTCCCTTCAATACTAGCGTGGTC |
| | TATAAAAAAACCTTGTTCGTAGAGTTTACGGACCATCTCTTCAACATT |
| | GCCAAACCTAGACCACCTTGGATGGGACTGCTCGGGCCGACCATACAG |
| | GCGGAAGTTTATGACACTGTTGTGATTACTTTGAAGAACATGGCTTCT |
| | CATCCGGTCAGTTTGCATGCGGTGGGTGTAAGTTACTGGAAGGCTTCT |
| | GAAGGCGCGGAGTATGATGACCAGACGTCCCAGCGAGAAAAAGAAGAC |
| | GATAAAGTTTTTCCAGGTGGGAGTCATACATATGTCTGGCAGGTCCTC |
| | AAGGAGAACGGCCCGATGGCGTCCGATCCGCTTTGCCTCACCTACTCC |
| | TACCTCTCACATGTTGACCTTGTAAAAGATTTGAACTCAGGTTTGATA |
| | GGTGCCCTTTTGGTGTGCCGGGAAGGTTCCCTCGCAAAAGAGAAAACG |
| | CAGACTCTTCACAAATTCATATTGCTGTTCGCAGTATTCGATGAAGGT |
| | AAGTCCTGGCACAGCGAAACCAAGAATTCACTGATGCAGGACAGAGAC |
| | GCTGCAAGCGCCCGAGCCTGGCCTAAGATGCATACTGTTAATGGGTAC |
| | GTTAACAGGTCACTCCCCGGGTTGATTGGTTGTCATAGGAAATCTGTA |
| | TACTGGCATGTTATAGGGATGGGCACAACACCCGAAGTGCATTCCATC |
| | TTCCTTGAAGGGCATACATTCCTGGTACGGAATCACCGGCAGGCCTCT |
| | TTGGAAATCTCTCCGATAACTTTTTTGACCGCTCAGACCCTTTTGATG |
| | GACCTCGGTCAGTTTCTTCTGTTCTGCCACATATCCAGCCATCAACAC |
| | GACGGGATGGAGGCATATGTCAAGGTCGATAGCTGTCCCGAAGAGCCC |
| | CAACTTCGCATGAAGAATAATGAAGAAGCCGAAGACTACGACGATGAT |
| | TTGACCGATTCCGAAATGGACGTTGTGCGATTCGACGATGACAACAGC |
| | CCTTCTTTCATCCAAATTAGATCAGTGGCTAAAAAGCACCCGAAAACC |
| | TGGGTGCACTATATCGCGGCAGAAGAAGAGGATTGGGACTACGCCCCC |
| | CTGGTCCTGGCGCCGGATGATCGAAGCTACAAGTCCCAGTATCTCAAC |
| | AACGGCCCTCAAAGGATCGGCCGGAAGTACAAGAAAGTTCGCTTCATG |
| | GCCTATACAGATGAAACCTTCAAAACCAGAGAAGCGATTCAGCATGAA |
| | AGTGGGATTCTCGGTCCACTTCTTTACGGGGAAGTTGGAGACACCCTC |
| | CTTATTATCTTTAAGAACCAAGCGAGTCGGCCTTACAACATCTATCCG |
| | CATGGGATAACCGACGTACGCCCACTTTACTCTCGAAGGTTGCCAAAG |
| | GGTGTTAAACATCTTAAAGACTTCCCGATTCTCCCTGGCGAAATATTC |
| | AAATATAAATGGACAGTGACGGTAGAAGATGGTCCCACCAAATCAGAC |
| | CCGAGGTGCTTGACCAGATATTATTCCTCTTTCGTAAATATGGAGAGG |
| | GATCTGGCCTCTGGTCTGATAGGTCCACTGCTGATTTGCTACAAGGAA |
| | TCAGTAGATCAAAGGGGCAACCAAATAATGTCCGATAAACGAAACGTC |
| | ATCTTGTTTAGTGTTTTCGATGAAAATCGGAGTTGGTATTTGACGGAG |
| | AACATTCAGCGCTTTCTCCCGAACCCCGCAGGGGTGCAACTGGAGGAT |
| | CCAGAATTCCAGGCATCTAACATAATGCATTCCATAAACGGCTATGTC |
| | TTTGACTCTCTCCAATTGAGTGTATGTCTGCATGAGGTCGCATACTGG |
| | TATATCCTCTCCATTGGGGCTCAAACCGATTTCTTGAGCGTCTTCTTC |
| | AGTGGATACACATTTAAACATAAAATGGTCTATGAGGATACCCTGACT |
| | CTTTTTCCCTTCTCTGGAGAAACCGTATTTATGTCCATGGAGAATCCT |
| | GGCTTGTGGATCCTCGGGTGTCATAACTCTGACTTCCGAAACCGAGGC |
| | ATGACGGCGCTGCTGAAGGTTTCTTCTTGCGATAAAAACACTGGGGAT |
| | TATTACGAGGACTCATATGAGGACATCTCCGCGTATCTTCTTAGCAAA |
| | AATAACGCGATTGAGCCTAGGAGTTTCAGTCAAAACAGCCGACACCCT |
| | AGTCAAAACCCACCAGTTCTCAAGCGGCATCAGCGCGAGATTACACGC |
| | ACCACGCTTCAGAGTGACCAGGAGGAAATTGATTATGACGACACCATC |
| | AGTGTCGAGATGAAGAAGGAGGACTTTGATATATATGACGAAGATGAA |
| | AATCAGAGCCCCAGAAGTTTTCAAAAGAAGACGAGGCATTATTTCATC |
| | GCAGCGGTTGAACGGCTCTGGGATTATGGGATGAGCAGCAGTCCGCAC |
| | GTACTCAGAAACAGAGCGCAGAGCGGGAGCGTACCTCAATTTAAGAAA |
| | GTTGTTTTTCAGGAATTTACCGACGGTTCTTTCACACAGCCTTTGTAT |
| | AGAGGGGAGCTCAATGAGCACCTTGGTTTGCTGGGACCTTATATAAGA |
| | GCCGAAGTTGAAGATAATATTATGGTGACGTTCCGGAACCAAGCATCC |
| | CGGCCCTACAGCTTCTACTCCTCTCTCATCTCTTACGAAGAAGATCAG |
| | CGGCAGGGAGCAGAACCCCGCAAAAACTTCGTGAAACCCAATGAGACA |
| | AAAACGTATTTCTGGAAAGTTCAGCACCACATGGCGCCCACCAAGGAT |
| | GAGTTTGACTGCAAGGCCTGGGCTTATTTTAGTGATGTAGATCTCGAA |
| | AAAGACGTGCATAGTGGGTTGATAGGGCCCCTGTTGGTATGTCACACG |
| | AATACTTTGAACCCAGCCCACGGTCGCCAGGTTACGGTACAAGAGTTT |
| | GCGCTCTTCTTCACGATATTTGATGAGACAAAGAGCTGGTATTTCACG |
| | GAGAATATGGAGAGAAACTGCCGGGCGCCTTGTAACATACAAATGGAA |
| | GATCCTACGTTCAAAGAGAACTACCGATTCCATGCTATAAATGGGTAT |
| | ATAATGGACACCCTTCCCGGGTTGGTCATGGCCCAAGATCAGCGCATT |
| | CGCTGGTACTTGCTTAGCATGGGCAGTAATGAGAACATCCATTCAATC |
| | CATTTCTCCGGTCACGTTTTCACGGTAAGAAAGAAAGAGGAGTAGAAA |
| | ATGGCTTTGTACAATCTGTATCCGGGGGTGTTCGAAACGGTCGAGATG |
| | CTGCCCAGTAAAGCGGGGATCTGGAGGGTCGAATGTCTTATCGGCGAG |
| | CATTTGCATGCGGGCATGTCTACTCTGTTCCTGGTGTACAGCAATAAA |
| | TGTCAAACGCCGTTGGGAATGGCATCCGGGCATATCAGGGACTTCCAG |
| | ATAACGGCTTCCGGGCAATACGGGCAATGGGCACCAAAGCTTGCAAGA |
| | CTCCACTACTCCGGGTCCATAAACGCGTGGTCTACTAAGGAGCCTTTC |
| | TCCTGGATCAAAGTAGATCTTCTGGCTCCCATGATTATACACGGCATT |
| | AAAACGCAAGGCGCGCGCCAAAAATTTAGCTCCCTCTATATATCCCAG |
| | TTTATTATCATGTATAGTCTCGATGGAAAGAAATGGCAAACTTACCGA |
| | GGTAACTCTACTGGTACCCTCATGGTATTTTTTGGGAACGTCGACTCA |
| | AGTGGCATTAAGCATAATATATTTAACCCGCCGATAATAGCTCGCTAC |
| | ATACGGTTGCATCCCACTCACTATAGCATACGCAGTACACTTAGGATG |
| | GAACTCATGGGTTGCGATCTGAACTCTTGTTCCATGCCTTTGGGGATG |
| | GAGTCCAAAGCGATATCCGAGGCTCAAATAACAGCCTCAAGTTATTTT |
| | ACAAACATGTTTGCGACGTGGAGTCCTAGCAAGGCAAGGCTCCACCTC |
| | CAAGGCCGAAGTAATGCTTGGCGGCCGCAGGTGAACAACCCCAAAGAA |
| | TGGTTGCAAGTGGATTTCCAAAAGACCATGAAAGTTACGGGTGTAACA |
| | ACACAGGGCGTTAAAAGTTTGCTTACCTCCATGTACGTTAAAGAGTTC |
| | TTGATATCATCTAGTCAGGATGGGCATCAATGGACCCTTTTCTTTCAA |
| | AACGGGAAGGTCAAAGTGTTTCAGGGCAATCAGGACTCTTTCACTCCC |
| | GTTGTCAACTCCCTCGACCCACCCCTGCTCACCAGGTATTTGAGAATA |
| | CATCCGCAGAGTTGGGTACACCAAATCGCACTCAGGATGGAGGTGCTC |
| | GGCTGTGAGGCACAGGACTTGTATTGA (SEQ ID NO: 55) |
| VWF | ATGATTCCTGCCAGATTTGCCGGGGTGCTGCTTGCTCTGGCCCTCATT |
| | TTGCCAGGGACCCTTTGTGCAGAAGGAACTCGCGGCAGGTCATCCACG |
| | GCCCGATGCAGCCTTTTCGGAAGTGACTTCGTCAACACCTTTGATGGG |
| | AGCATGTACAGCTTTGCGGGATACTGCAGTTACCTCCTGGCAGGGGGC |
| | TGCCAGAAACGCTCCTTCTCGATTATTGGGGACTTCCAGAATGGCAAG |
| | AGAGTGAGCCTCTCCGTGTATCTTGGGGAATTTTTTGACATCCATTTG |
| | TTTGTCAATGGTACCGTGACACAGGGGGACCAAAGAGTCTCCATGCCC |
| | TATGCCTCCAAAGGGCTGTATCTAGAAACTGAGGCTGGGTACTACAAG |
| | CTGTCCGGTGAGGCCTATGGCTTTGTGGCCAGGATCGATGGCAGCGGC |
| | AACTTTCAAGTCCTGCTGTCAGACAGATACTTCAACAAGACCTGCGGG |
| | CTGTGTGGCAACTTTAACATCTTTGCTGAAGATGACTTTATGACCCAA |
| | GAAGGGACCTTGACCTCGGACCCTTATGACTTTGCCAACTCATGGGCT |
| | CTGAGCAGTGGAGAACAGTGGTGTGAACGGGCATCTCCTCCCAGCAGC |
| | TCATGCAACATCTCCTCTGGGGAAATGCAGAAGGGCCTGTGGGAGCAG |
| | TGCCAGCTTCTGAAGAGCACCTCGGTGTTTGCCCGCTGCCACCCTCTG |
| | GTGGACCCCGAGCCTTTTGTGGCCCTGTGTGAGAAGACTTTGTGTGAG |
| | TGTGCTGGGGGGCTGGAGTGCGCCTGCCCTGCCCTCCGGAGTACGCCC |
| | GGACCTGTGCCCAGGAGGGAATGGTGCTGTACGGCTGGACCGACCACA |
| | GCGCGTGCAGCCCAGTGTGCCCTGCTGGTATGGAGTATAGGCAGTGTG |
| | TGTCCCCTTGCGCCAGGACCTGCCAGAGCCTGCACATCAATGAAATGT |
| | GTCAGGAGCGATGCGTGGATGGCTGCAGCTGCCCTGAGGGACAGCTCC |
| | TGGATGAAGGCCTCTGCGTGGAGAGCACCGAGTGTCCCTGCGTGCATT |
| | CCGGAAAGCGCTACCCTCCCGGCACCTCCCTCTCTCGAGACTGCAACA |
| | CCTGCATTTGCCGAAACAGCCAGTGGATCTGCAGCAATGAAGAATGTC |
| | CAGGGGAGTGCCTTGTCACAGGTCAATCACACTTCAAGAGCTTTGACA |
| | ACAGATACTTCACCTTCAGTGGGATCTGCCAGTACCTGCTGGCCCGGG |
| | ATTGCCAGGACCACTCCTTCTCCATTGTCATTGAGACTGTCCAGTGTG |
| | CTGATGACCGCGACGCTGTGTGCACCCGCTCCGTCACCGTCCGGCTGC |
| | CTGGCCTGCACAACAGCCTTGTGAAACTGAAGCATGGGGCAGGAGTTG |
| | CCATGGATGGCCAGGACGTCCAGCTCCCCCTCCTGAAAGGTGACCTCC |
| | GCATCCAGCATACAGTGACGGCCTCCGTGCGCCTCAGCTACGGGGAGG |
| | ACCTGCAGATGGACTGGGATGGCCGCGGGAGGCTGCTGGTGAAGCTGT |
| | CCCCCGTCTATGCCGGGAAGACCTGCGGCCTGTGTGGGAATTACAATG |
| | GCAACCAGGGCGACGACTTCCTTACCCCCTCTGGGCTGGCGGAGCCCC |
| | GGGTGGAGGACTTCGGGAACGCCTGGAAGCTGCACGGGGACTGCCAGG |
| | ACCTGCAGAAGCAGCACAGCGATCCCTGCGCCCTCAACCCGCGCATGA |
| | CCAGGTTCTCCGAGGAGGCGTGCGCGGTCCTGACGTCCCCCACATTCG |
| | AGGCCTGCCATCGTGCCGTCAGCCCGCTGCCCTACCTGCGGAACTGCC |
| | GCTACGACGTGTGCTCCTGCTCGGACGGCCGCGAGTGCCTGTGCGGCG |
| | CCCTGGCCAGCTATGCCGCGGCCTGCGCGGGGAGAGGCGTGCGCGTCG |
| | CGTGGCGCGAGCCAGGCCGCTGTGAGCTGAACTGCCCGAAAGGCCAGG |
| | TGTACCTGCAGTGCGGGACCCCCTGCAACCTGACCTGCCGCTCTCTCT |
| | CTTACCCGGATGAGGAATGCAATGAGGCCTGCCTGGAGGGCTGCTTCT |
| | GCCCCCCAGGGCTCTACATGGATGAGAGGGGGGACTGCGTGCCCAAGG |
| | CCCAGTGCCCCTGTTACTATGACGGTGAGATCTTCCAGCCAGAAGACA |
| | TCTTCTCAGACCATCACACCATGTGCTACTGTGAGGATGGCTTCATGC |
| | ACTGTACCATGAGTGGAGTCCCCGGAAGCTTGCTGCCTGACGCTGTCC |
| | TCAGCAGTCCCCTGTCTCATCGCAGCAAAAGGAGCCTATCCTGTCGGC |
| | CCCCCATGGTCAAGCTGGTGTGTCCCGCTGACAACCTGCGGGCTGAAG |
| | GGCTCGAGTGTACCAAAACGTGCCAGAACTATGACCTGGAGTGCATGA |
| | GCATGGGCTGTGTCTCTGGCTGCCTCTGCCCCCCGGGCATGGTCCGGC |
| | ATGAGAACAGATGTGTGGCCCTGGAAAGGTGTCCCTGCTTCCATCAGG |
| | GCAAGGAGTATGCCCCTGGAGAAACAGTGAAGATTGGCTGCAACACTT |
| | GTGTCTGTCGGGACCGGAAGTGGAACTGCACAGACCATGTGTGTGATG |
| | CCACGTGCTCCACGATCGGCATGGCCCACTACCTCACCTTCGACGGGC |
| | TCAAATACCTGTTCCCCGGGGAGTGCCAGTACGTTCTGGTGCAGGATT |
| | ACTGCGGCAGTAACCCTGGGACCTTTCGGATCCTAGTGGGGAATAAGG |
| | GATGCAGCCACCCCTCAGTGAAATGCAAGAAACGGGTCACCATCCTGG |
| | TGGAGGGAGGAGAGATTGAGCTGTTTGACGGGGAGGTGAATGTGAAGA |
| | GGCCCATGAAGGATGAGACTCACTTTGAGGTGGTGGAGTCTGGCCGGT |
| | ACATCATTCTGCTGCTGGGCAAAGCCCTCTCCGTGGTCTGGGACCGCC |
| | ACCTGAGCATCTCCGTGGTCCTGAAGCAGACATACCAGGAGAAAGTGT |
| | GTGGCCTGTGTGGGAATTTTGATGGCATCCAGAACAATGACCTCACCA |
| | GCAGCAACCTCCAAGTGGAGGAAGACCCTGTGGACTTTGGGAACTCCT |
| | GGAAAGTGAGCTCGCAGTGTGCTGACACCAGAAAAGTGCCTCTGGACT |
| | CATCCCCTGCCACCTGCCATAACAACATCATGAAGCAGACGATGGTGG |
| | ATTCCTCCTGTAGAATCCTTACCAGTGACGTCTTCCAGGACTGCAACA |
| | AGCTGGTGGACCCCGAGCCATATCTGGATGTCTGCATTTACGACACCT |
| | GCTCCTGTGAGTCCATTGGGGACTGCGCCTGCTTCTGCGACACCATTG |
| | CTGCCTATGCCCACGTGTGTGCCCAGCATGGCAAGGTGGTGACCTGGA |
| | GGACGGCCACATTGTGCCCCCAGAGCTGCGAGGAGAGGAATCTCCGGG |
| | AGAACGGGTATGAGTGTGAGTGGCGCTATAACAGCTGTGCACCTGCCT |
| | GTCAAGTCACGTGTCAGCACCCTGAGCCACTGGCCTGCCCTGTGCAGT |
| | GTGTGGAGGGCTGCCATGCCCACTGCCCTCCAGGGAAAATCCTGGATG |
| | AGCTTTTGCAGACCTGCGTTGACCCTGAAGACTGTCCAGTGTGTGAGG |
| | TGGCTGGCCGGCGTTTTGCCTCAGGAAAGAAAGTCACCTTGAATCCCA |
| | GTGACCCTGAGCACTGCCAGATTTGCCACTGTGATGTTGTCAACCTCA |
| | CCTGTGAAGCCTGCCAGGAGCCGGGAGGCCTGGTGGTGCCTCCCACAG |
| | ATGCCCCGGTGAGCCCCACCACTCTGTATGTGGAGGACATCTCGGAAC |
| | CGCCGTTGCACGATTTCTACTGCAGCAGGCTACTGGACCTGGTCTTCC |
| | TGCTGGATGGCTCCTCCAGGCTGTCCGAGGCTGAGTTTGAAGTGCTGA |
| | AGGCCTTTGTGGTGGACATGATGGAGCGGCTGCGCATCTCCCAGAAGT |
| | GGGTCCGCGTGGCCGTGGTGGAGTACCACGACGGCTCCCACGCCTACA |
| | TCGGGCTCAAGGACCGGAAGCGACCGTCAGAGCTGCGGCGCATTGCCA |
| | GCCAGGTGAAGTATGCGGGCAGCCAGGTGGCCTCCACCAGCGAGGTCT |
| | TGAAATACACACTGTTCCAAATCTTCAGCAAGATCGACCGCCCTGAAG |
| | CCTCCCGCATCACCCTGCTCCTGATGGCCAGCCAGGAGCCCCAACGGA |
| | TGTCCCGGAACTTTGTCCGCTACGTCCAGGGCCTGAAGAAGAAGAAGG |
| | TCATTGTGATCCCGGTGGGCATTGGGCCCCATGCCAACCTCAAGCAGA |
| | TCCGCCTCATCGAGAAGCAGGCCCCTGAGAACAAGGCCTTCGTGCTGA |
| | GCAGTGTGGATGAGCTGGAGCAGCAAAGGGACGAGATCGTTAGCTACC |
| | TCTGTGACCTTGCCCCTGAAGCCCCTCCTCCTACTCTGCCCCCCGACA |
| | TGGCACAAGTCACTGTGGGCCCGGGGCTCTTGGGGGTTTCGACCCTGG |
| | GGCCCAAGAGGAACTCCATGGTTCTGGATGTGGCGTTCGTCCTGGAAG |
| | GATCGGACAAAATTGGTGAAGCCGACTTCAACAGGAGCAAGGAGTTCA |
| | TGGAGGAGGTGATTCAGCGGATGGATGTGGGCCAGGACAGCATCCACG |
| | TCACGGTGCTGCAGTACTCCTACATGGTGACTGTGGAGTACCCCTTCA |
| | GCGAGGCACAGTCCAAAGGGGACATCCTGCAGCGGGTGCGAGAGATCC |
| | GCTACCAGGGCGGCAACAGGACCAACACTGGGCTGGCCCTGCGGTACC |
| | TCTCTGACCACAGCTTCTTGGTCAGCCAGGGTGACCGGGAGCAGGCGC |
| | CCAACCTGGTCTACATGGTCACCGGAAATCCTGCCTCTGATGAGATCA |
| | AGAGGCTGCCTGGAGACATCCAGGTGGTGCCCATTGGAGTGGGCCCTA |
| | ATGCCAACGTGCAGGAGCTGGAGAGGATTGGCTGGCCCAATGCCCCTA |
| | TCCTCATCCAGGACTTTGAGACGCTCCCCCGAGAGGCTCCTGACCTGG |
| | TGCTGCAGAGGTGCTGCTCCGGAGAGGGGCTGCAGATCCCCACCCTCT |
| | CCCCTGCACCTGACTGCAGCCAGCCCCTGGACGTGATCCTTCTCCTGG |
| | ATGGCTCCTCCAGTTTCCCAGCTTCTTATTTTGATGAAATGAAGAGTT |
| | TCGCCAAGGCTTTCATTTCAAAAGCCAATATAGGGCCTCGTCTCACTC |
| | AGGTGTCAGTGCTGCAGTATGGAAGCATCACCACCATTGACGTGCCAT |
| | GGAACGTGGTCCCGGAGAAAGCCCATTTGCTGAGCCTTGTGGACGTCA |
| | TGCAGCGGGAGGGAGGCCCCAGCCAAATCGGGGATGCCTTGGGCTTTG |
| | CTGTGCGATACTTGACTTCAGAAATGCATGGTGCCAGGCCGGGAGCCT |
| | CAAAGGCGGTGGTCATCCTGGTCACGGACGTCTCTGTGGATTCAGTGG |
| | ATGCAGCAGCTGATGCCGCCAGGTCCAACAGAGTGACAGTGTTCCCTA |
| | TTGGAATTGGAGATCGCTACGATGCAGCCCAGCTACGGATCTTGGCAG |
| | GCCCAGCAGGCGACTCCAACGTGGTGAAGCTCCAGCGAATCGAAGACC |
| | TCCCTACCATGGTCACCTTGGGCAATTCCTTCCTCCACAAACTGTGCT |
| | CTGGATTTGTTAGGATTTGCATGGATGAGGATGGGAATGAGAAGAGGC |
| | CCGGGGACGTCTGGACCTTGCCAGACCAGTGCCACACCGTGACTTGCC |
| | AGCCAGATGGCCAGACCTTGCTGAAGAGTCATCGGGTCAACTGTGACC |
| | GGGGGCTGAGGCCTTCGTGCCCTAACAGCCAGTCCCCTGTTAAAGTGG |
| | AAGAGACCTGTGGCTGCCGCTGGACCTGCCCCTGCGTGTGCACAGGCA |
| | GCTCCACTCGGCACATCGTGACCTTTGATGGGCAGAATTTCAAGCTGA |
| | CTGGCAGCTGTTCTTATGTCCTATTTCAAAACAAGGAGCAGGACCTGG |
| | AGGTGATTCTCCATAATGGTGCCTGCAGCCCTGGAGCAAGGCAGGGCT |
| | GCATGAAATCCATCGAGGTGAAGCACAGTGCCCTCTCCGTCGAGCTGC |
| | ACAGTGACATGGAGGTGACGGTGAATGGGAGACTGGTCTCTGTTCCTT |
| | ACGTGGGTGGGAACATGGAAGTCAACGTTTATGGTGCCATCATGCATG |
| | AGGTCAGATTCAATCACCTTGGTGAGATCTTGAGATTGAGTCCACAAA |
| | ACAATGAGTTCCAACTGGAGCTGAGCCCCAAGACTTTTGCTTCAAAGA |
| | CGTATGGTCTGTGTGGGATCTGTGATGAGAACGGAGCCAATGACTTCA |
| | TGCTGAGGGATGGCACAGTCACCACAGACTGGAAAACACTTGTTCAGG |
| | AATGGACTGTGCAGCGGCCAGGGCAGACGTGCCAGCCCATCCTGGAGG |
| | AGCAGTGTCTTGTCCCCGACAGCTCCCACTGCCAGGTCCTCCTCTTAC |
| | CACTGTTTGCTGAATGCCACAAGGTCCTGGCTCCAGCCACATTCTATG |
| | CCATCTGCCAGCAGGACAGTTGCCACCAGGAGCAAGTGTGTGAGGTGA |
| | TCGCCTCTTATGCCCACCTCTGTCGGACCAACGGGGTCTGCGTTGACT |
| | GGAGGACACCTGATTTCTGTGCTATGTCATGCCCACCATCTCTGGTCT |
| | ACAACCACTGTGAGCATGGCTGTCCCCGGCACTGTGATGGCAACGTGA |
| | GCTCCTGTGGGGACCATCCCTCCGAAGGCTGTTTCTGCCCTCCAGATA |
| | AAGTCATGTTGGAAGGCAGCTGTGTCCCTGAAGAGGCCTGCACTCAGT |
| | GCATTGGTGAGGATGGAGTCCAGCACCAGTTCCTGGAAGCCTGGGTCC |
| | CGGACCACCAGCCCTGTCAGATCTGCACATGCCTCAGCGGGCGGAAGG |
| | TCAACTGCACAACGCAGCCCTGCCCCACGGCCAAAGCTCCCACGTGTG |
| | GCCTGTGTGAAGTAGCCCGCCTCCGCCAGAATGCAGACCAGTGCTGCC |
| | CCGAGTATGAGTGTGTGTGTGACCCAGTGAGCTGTGACCTGCCCCCAG |
| | TGCCTCACTGTGAACGTGGCCTCCAGCCCACACTGACCAACCCTGGCG |
| | AGTGCAGACCCAACTTCACCTGCGCCTGCAGGAAGGAGGAGTGCAAAA |
| | GAGTGTCCCCACCCTCCTGCCCCCCGCACCGTTTGCCCACCCTTCGGA |
| | AGACCCAGTGCTGTGATGAGTATGAGTGTGCCTGCAACTGTGTCAACT |
| | CCACAGTGAGCTGTCCCCTTGGGTACTTGGCCTCAACTGCCACCAATG |
| | ACTGTGGCTGTACCACAACCACCTGCCTTCCCGACAAGGTGTGTGTCC |
| | ACCGAAGCACCATCTACCCTGTGGGCCAGTTCTGGGAGGAGGGCTGCG |
| | ATGTGTGCACCTGCACCGACATGGAGGATGCCGTGATGGGCCTCCGCG |
| | TGGCCCAGTGCTCCCAGAAGCCCTGTGAGGACAGCTGTCGGTCGGGCT |
| | TCACTTACGTTCTGCATGAAGGCGAGTGCTGTGGAAGGTGCCTGCCAT |
| | CTGCCTGTGAGGTGGTGACTGGCTCACCGCGGGGGGACTCCCAGTCTT |
| | CCTGGAAGAGTGTCGGCTCCCAGTGGGCCTCCCCGGAGAACCCCTGCC |
| | TCATCAATGAGTGTGTCCGAGTGAAGGAGGAGGTCTTTATACAACAAA |
| | GGAACGTCTCCTGCCCCCAGCTGGAGGTCCCTGTCTGCCCCTCGGGCT |
| | TTCAGCTGAGCTGTAAGACCTCAGCGTGCTGCCCAAGCTGTCGCTGTG |
| | AGCGCATGGAGGCCTGCATGCTCAATGGCACTGTCATTGGGCCCGGGA |
| | AGACTGTGATGATCGATGTGTGCACGACCTGCCGCTGCATGGTGCAGG |
| | TGGGGGTCATCTCTGGATTCAAGCTGGAGTGCAGGAAGACCACCTGCA |
| | ACCCCTGCCCCCTGGGTTACAAGGAAGAAAATAACACAGGTGAATGTT |
| | GTGGGAGATGTTTGCCTACGGCTTGCACCATTCAGCTAAGAGGAGGAC |
| | AGATCATGACACTGAAGCGTGATGAGACGCTCCAGGATGGCTGTGATA |
| | CTCACTTCTGCAAGGTCAATGAGAGAGGAGAGTAGTTCTGGGAGAAGA |
| | GGGTCACAGGCTGCCCACCCTTTGATGAACACAAGTGTCTGGCTGAGG |
| | GAGGTAAAATTATGAAAATTCCAGGCACCTGCTGTGACACATGTGAGG |
| | AGCCTGAGTGCAACGACATCACTGCCAGGCTGCAGTATGTCAAGGTGG |
| | GAAGCTGTAAGTCTGAAGTAGAGGTGGATATCCACTACTGCCAGGGCA |
| | AATGTGCCAGCAAAGCCATGTACTCCATTGACATCAACGATGTGCAGG |
| | ACCAGTGCTCCTGCTGCTCTCCGACACGGACGGAGCCCATGCAGGTGG |
| | CCCTGCACTGCACCAATGGCTCTGTTGTGTACCATGAGGTTCTCAATG |
| | CCATGGAGTGCAAATGCTCCCCCAGGAAGTGCAGCAAGTGA (SEQ |
| | ID NO: 56) |
| GCG | ATGAAAAGCATTTACTTTGTGGCTGGATTATTTGTAATGCTGGTACAA |
| | GGCAGCTGGCAACGTTCCCTTCAAGACACAGAGGAGAAATCCAGATCA |
| | TTCTCAGCTTCCCAGGCAGACCCACTCAGTGATCCTGATCAGATGAAC |
| | GAGGACAAGCGCCATTCACAGGGCACATTCACCAGTGACTACAGCAAG |
| | TATCTGGACTCCAGGCGTGCCCAAGATTTTGTGCAGTGGTTGATGAAT |
| | ACCAAGAGGAACAGGAATAACATTGCCAAACGTCACGATGAATTTGAG |
| | AGACATGCTGAAGGGACCTTTACCAGTGATGTAAGTTCTTATTTGGAA |
| | GGCCAAGCTGCCAAGGAATTCATTGCTTGGCTGGTGAAAGGCCGAGGA |
| | AGGCGAGATTTCCCAGAAGAGGTCGCCATTGTTGAAGAACTTGGCCGC |
| | AGACATGCTGATGGTTCTTTCTCTGATGAGATGAACACCATTCTTGAT |
| | AATCTTGCCGCCAGGGACTTTATAAACTGGTTGATTCAGACCAAAATC |
| | ACTGACAGGAAATAA (SEQ ID NO: 57) |
| Factor IX | ATGCAGCGCGTGAACATGATCATGGCAGAATCACCAGGCCTCATCACC |
| | ATCTGCCTTTTAGGATATCTACTCAGTGCTGAATGTACAGTTTTTCTT |
| | GATCATGAAAACGCCAACAAAATTCTGAATCGGCCAAAGAGGTATAAT |
| | TCAGGTAAATTGGAAGAGTTTGTTCAAGGGAACCTTGAGAGAGAATGT |
| | ATGGAAGAAAAGTGTAGTTTTGAAGAAGCACGAGAAGTTTTTGAAAAC |
| | ACTGAAAGAACAACTGAATTTTGGAAGCAGTATGTTGATGGAGATGAG |
| | TGTGAGTCCAATCCATGTTTAAATGGCGGCAGTTGCAAGGATGACATT |
| | AATTCCTATGAATGTTGGTGTCCCTTTGGATTTGAAGGAAAGAACTGT |
| | GAATTAGATGTAACATGTAACATTAAGAATGGCAGATGCGAGGAGTTT |
| | TGTAAAAATAGTGCTGATAACAAGGTGGTTTGCTCCTGTACTGAGGGA |
| | TATCGACTTGCAGAAAACCAGAAGTCCTGTGAACCAGCAGTGCCATTT |
| | CCATGTGGAAGAGTTTCTGTTTCACAAACTTCTAAGCTCACCCGTGCT |
| | GAGACTGTTTTTCCTGATGTGGACTATGTAAATTCTACTGAAGCTGAA |
| | ACCATTTTGGATAACATCACTCAAAGCACCCAATCATTTAATGACTTC |
| | ACTCGGGTTGTTGGTGGAGAAGATGCCAAACCAGGTCAATTCCCTTGG |
| | CAGGTTGTTTTGAATGGTAAAGTTGATGCATTCTGTGGAGGCTCTATC |
| | GTTAATGAAAAATGGATTGTAACTGCTGCCCACTGTGTTGAAACTGGT |
| | GTTAAAATTACAGTTGTCGCAGGTGAACATAATATTGAGGAGACAGAA |
| | CATACAGAGCAAAAGCGAAATGTGATTCGAATTATTCCTCACCACAAC |
| | TACAATGCAGCTATTAATAAGTACAACCATGACATTGCCCTTCTGGAA |
| | CTGGACGAACCCTTAGTGCTAAACAGCTACGTTACACCTATTTGCATT |
| | GCTGACAAGGAATACACGAACATCTTCCTCAAATTTGGATCTGGCTAT |
| | GTAAGTGGCTGGGGAAGAGTCTTCCACAAAGGGAGATCAGCTTTAGTT |
| | CTTCAGTACCTTAGAGTTCCACTTGTTGACCGAGCCACATGTCTTCGA |
| | TCTACAAAGTTCACCATCTATAACAACATGTTCTGTGCTGGCTTCCAT |
| | GAAGGAGGTAGAGATTCATGTCAAGGAGATAGTGGGGGACCCCATGTT |
| | ACTGAAGTGGAAGGGACCAGTTTCTTAACTGGAATTATTAGCTGGGGT |
| | GAAGAGTGTGCAATGAAAGGCAAATATGGAATATATACCAAGGTATCC |
| | CGGTATGTCAACTGGATTAAGGAAAAAACAAAGCTCACTTAA (SEQ |
| | ID NO: 58) |
| Proenkephalin | GGGACGGCGAGGCAGGCGCTCAGAGCCCCGCAGCCTGGCCCGTGACCC |
| | CGCAGAGACGCTGAGGACCGCGACGAGTCGTGTCTGAACCCGGCTTTT |
| | CCAATTGGCCTGCTCCATCCGAACAGCGTCAACTCCATGGCGCGGTTC |
| | CTGACACTTTGCACTTGGCTGCTGTTGCTCGGCCCCGGGCTCCTGGCG |
| | ACCGTGCGGGCCGAATGCAGCCAGGATTGCGCGACGTGCAGCTACCGC |
| | CTAGTGCGCCCGGCCGACATCAACTTCCTGGCTTGCGTAATGGAATGT |
| | GAAGGTAAACTGCCTTCTCTGAAAATTTGGGAAACCTGCAAGGAGCTC |
| | CTGCAGCTGTCCAAACCAGAGCTTCCTCAAGATGGCACCAGCACCCTC |
| | AGAGAAAATAGCAAACCGGAAGAAAGCCATTTGCTAGCCAAAAGGTAT |
| | GGGGGCTTCATGAAAAGGTATGGAGGCTTCATGAAGAAAATGGATGAG |
| | CTTTATCCCATGGAGCCAGAAGAAGAGGCCAATGGAAGTGAGATCCTC |
| | GCCAAGCGGTATGGGGGCTTCATGAAGAAGGATGCAGAGGAGGACGAC |
| | TCGCTGGCCAATTCCTCAGACCTGCTAAAAGAGCTTCTGGAAACAGGG |
| | GACAACCGAGAGCGTAGCCACCACCAGGATGGCAGTGATAATGAGGAA |
| | GAAGTGAGCAAGAGATATGGGGGCTTCATGAGAGGCTTAAAGAGAAGC |
| | CCCCAACTGGAAGATGAAGCCAAAGAGCTGCAGAAGCGATATGGGGGC |
| | TTCATGAGAAGAGTAGGTCGCCCAGAGTGGTGGATGGACTACCAGAAA |
| | CGGTATGGAGGTTTCCTGAAGCGCTTTGCCGAGGCTCTGCCCTCCGAC |
| | GAAGAAGGCGAAAGTTACTCCAAAGAAGTTCCTGAAATGGAAAAAAGA |
| | TACGGAGGATTTATGAGATTTTAATATCTTTTCCCACTAGTGGCCCCA |
| | GGCCCCAGCAAGCCTCCCTCCATCCTCCAGTGGGAAACTGTTGATGGT |
| | GTTTTATTGTCATGTGTTGCTTGCCTTGTATAGTTGACTTCATTGTCT |
| | GGATAACTATACAACCTGAAAACTGTCATTTCAGGTTCTGTGCTCTTT |
| | TTGGAGTCTTTAAGCTCAGTATTAGTCTATTGCAGCTATCTCGTTTTC |
| | ATGCTAAAATAGTTTTTGTTATCTTGTCTCTTATTTTTGACAAACATC |
| | AATAAATGCTTACTTGTATATAGAGATAATAAACCTATTACCCCAAGT |
| | GCA (SEQ ID NO: 59) |
| EGF | ATGCTGCTCACTCTTATCATTCTGTTGCCAGTAGTTTCAAAATTTAGT |
| | TTTGTTAGTCTCTCAGCACCGCAGCACTGGAGCTGTCCTGAAGGTACT |
| | CTCGCAGGAAATGGGAATTCTACTTGTGTGGGTCCTGCACCCTTCTTA |
| | ATTTTCTCCCATGGAAATAGTATCTTTAGGATTGACACAGAAGGAACC |
| | AATTATGAGCAATTGGTGGTGGATGCTGGTGTCTCAGTGATCATGGAT |
| | TTTCATTATAATGAGAAAAGAATCTATTGGGTGGATTTAGAAAGACAA |
| | CTTTTGCAAAGAGTTTTTCTGAATGGGTCAAGGCAAGAGAGAGTATGT |
| | AATATAGAGAAAAATGTTTCTGGAATGGCAATAAATTGGATAAATGAA |
| | GAAGTTATTTGGTCAAATCAACAGGAAGGAATCATTACAGTAACAGAT |
| | ATGAAAGGAAATAATTCCCACATTCTTTTAAGTGCTTTAAAATATCCT |
| | GCAAATGTAGCAGTTGATCCAGTAGAAAGGTTTATATTTTGGTCTTCA |
| | GAGGTGGCTGGAAGCCTTTATAGAGCAGATCTCGATGGTGTGGGAGTG |
| | AAGGCTCTGTTGGAGACATCAGAGAAAATAACAGCTGTGTCATTGGAT |
| | GTGCTTGATAAGCGGCTGTTTTGGATTCAGTACAACAGAGAAGGAAGC |
| | AATTCTCTTATTTGCTCCTGTGATTATGATGGAGGTTCTGTCCACATT |
| | AGTAAACATCCAACACAGCATAATTTGTTTGCAATGTCCCTTTTTGGT |
| | GACCGTATCTTCTATTCAACATGGAAAATGAAGACAATTTGGATAGCC |
| | AACAAACACACTGGAAAGGACATGGTTAGAATTAACCTCCATTCATCA |
| | TTTGTACCACTTGGTGAACTGAAAGTAGTGCATCCACTTGCACAACCC |
| | AAGGCAGAAGATGACACTTGGGAGCCTGAGCAGAAACTTTGCAAATTG |
| | AGGAAAGGAAACTGCAGCAGCACTGTGTGTGGGCAAGACCTCCAGTCA |
| | CACTTGTGCATGTGTGCAGAGGGATACGCCCTAAGTCGAGACCGGAAG |
| | TACTGTGAAGATGTTAATGAATGTGCTTTTTGGAATCATGGCTGTACT |
| | CTTGGGTGTAAAAACACCCCTGGATCCTATTACTGCACGTGCCCTGTA |
| | GGATTTGTTCTGCTTCCTGATGGGAAACGATGTCATCAACTTGTTTCC |
| | TGTCCACGCAATGTGTCTGAATGCAGCCATGACTGTGTTCTGACATCA |
| | GAAGGTCCCTTATGTTTCTGTCCTGAAGGCTCAGTGCTTGAGAAAGAT |
| | GGGAAAACATGTAGCGGTTGTTCCTCACCCGATAATGGTGGATGTAGC |
| | CAGCTCTGCGTTCCTCTTAGCCCAGTATCCTGGGAATGTGATTGCTTT |
| | CCTGGGTATGACCTACAACTGGATGAAAAAAGCTGTGCAGCTTCAGGA |
| | CCACAACCATTTTTGCTGTTTGCCAATTCTCAAGATATTCGACACATG |
| | CATTTTGATGGAACAGACTATGGAACTCTGCTCAGCCAGCAGATGGGA |
| | ATGGTTTATGCCCTAGATCATGACCCTGTGGAAAATAAGATATACTTT |
| | GCCCATACAGCCCTGAAGTGGATAGAGAGAGCTAATATGGATGGTTCC |
| | CAGCGAGAAAGGCTTATTGAGGAAGGAGTAGATGTGCCAGAAGGTCTT |
| | GCTGTGGACTGGATTGGCCGTAGATTCTATTGGACAGACAGAGGGAAA |
| | TCTCTGATTGGAAGGAGTGATTTAAATGGGAAACGTTCCAAAATAATC |
| | ACTAAGGAGAACATCTCTCAACCACGAGGAATTGCTGTTCATCCAATG |
| | GCCAAGAGATTATTCTGGACTGATACAGGGATTAATCCACGAATTGAA |
| | AGTTCTTCCCTCCAAGGCCTTGGCCGTCTGGTTATAGCCAGCTCTGAT |
| | CTAATCTGGCCCAGTGGAATAACGATTGACTTCTTAACTGACAAGTTG |
| | TACTGGTGCGATGCCAAGCAGTCTGTGATTGAAATGGCCAATCTGGAT |
| | GGTTCAAAACGCCGAAGACTTACCCAGAATGATGTAGGTCACCCATTT |
| | GCTGTAGCAGTGTTTGAGGATTATGTGTGGTTCTCAGATTGGGCTATG |
| | CCATCAGTAATAAGAGTAAACAAGAGGACTGGCAAAGATAGAGTACGT |
| | CTCCAAGGCAGCATGCTGAAGCCCTCATCACTGGTTGTGGTTCATCCA |
| | TTGGCAAAACCAGGAGCAGATCCCTGCTTATATCAAAACGGAGGCTGT |
| | GAACATATTTGCAAAAAGAGGCTTGGAACTGCTTGGTGTTCGTGTCGT |
| | GAAGGTTTTATGAAAGCCTCAGATGGGAAAACGTGTCTGGCTCTGGTT |
| | GGTCATCAGCTGTTGGCAGGTGGTGAAGTTGATCTAAAGAACCAAGTA |
| | ACACCATTGGACATCTTGTCCAAGACTAGAGTGTCAGAAGATAACATT |
| | ACAGAATCTCAACACATGCTAGTGGCTGAAATCATGGTGTCAGATCAA |
| | GATGACTGTGCTCCTGTGGGATGCAGCATGTATGCTCGGTGTATTTCA |
| | GAGGGAGAGGATGCCACATGTCAGTGTTTGAAAGGATTTGCTGGGGAT |
| | GGAAAACTATGTTCTGATATAGATGAATGTGAGATGGGTGTCCCAGTG |
| | TGCCCCCCTGCCTCCTCCAAGTGCATCAACACCGAAGGTGGTTATGTC |
| | TGCCGGTGCTCAGAAGGCTACCAAGGAGATGGGATTCACTGTCTTGAT |
| | ATTGATGAGTGCCAACTGGGGGTGCACAGCTGTGGAGAGAATGCCAGC |
| | TGCACAAATACAGAGGGAGGCTATACCTGCATGTGTGCTGGACGCCTG |
| | TCTGAACCAGGACTGATTTGCCCTGACTCTACTCCACCCCCTCACCTC |
| | AGGGAAGATGACCACCACTATTCCGTAAGAAATAGTGACTCTGAATGT |
| | CCCCTGTCCCACGATGGGTACTGCCTCCATGATGGTGTGTGCATGTAT |
| | ATTGAAGCATTGGACAAGTATGCATGCAACTGTGTTGTTGGCTACATC |
| | GGGGAGCGATGTCAGTACCGAGACCTGAAGTGGTGGGAACTGCGCCAC |
| | GCTGGCCACGGGCAGCAGCAGAAGGTCATCGTGGTGGCTGTCTGCGTG |
| | GTGGTGCTTGTCATGCTGCTCCTCCTGAGCCTGTGGGGGGCCCACTAC |
| | TACAGGACTCAGAAGCTGCTATCGAAAAACCCAAAGAATCCTTATGAG |
| | GAGTCGAGCAGAGATGTGAGGAGTCGCAGGCCTGCTGACACTGAGGAT |
| | GGGATGTCCTCTTGCCCTCAACCTTGGTTTGTGGTTATAAAAGAACAC |
| | CAAGACCTCAAGAATGGGGGTCAACCAGTGGCTGGTGAGGATGGCCAG |
| | GCAGCAGATGGGTCAATGCAACCAACTTCATGGAGGCAGGAGCCCCAG |
| | TTATGTGGAATGGGCACAGAGCAAGGCTGCTGGATTCCAGTATCCAGT |
| | GATAAGGGCTCCTGTCCCCAGGTAATGGAGCGAAGCTTTCATATGCCC |
| | TCCTATGGGACACAGACCCTTGAAGGGGGTGTCGAGAAGCCCCATTCT |
| | CTCCTATCAGCTAACCCATTATGGCAACAAAGGGCCCTGGACCCACCA |
| | CACCAAATGGAGCTGACTCAGTGA (SEQ ID NO: 60) |
| IGF-1 | ATGGGAAAAATCAGCAGTCTTCCAACCCAATTATTTAAGTGCTGCTTT |
| | TGTGATTTCTTGAAGGTGAAGATGCACACCATGTCCTCCTCGCATCTC |
| | TTCTACCTGGCGCTGTGCCTGCTCACCTTCACCAGCTCTGCCACGGCT |
| | GGACCGGAGACGCTCTGCGGGGCTGAGCTGGTGGATGCTCTTCAGTTC |
| | GTGTGTGGAGACAGGGGCTTTTATTTCAACAAGCCCACAGGGTATGGC |
| | TCCAGCAGTCGGAGGGCGCCTCAGACAGGCATCGTGGATGAGTGCTGC |
| | TTCCGGAGCTGTGATCTAAGGAGGCTGGAGATGTATTGCGCACCCCTC |
| | AAGCCTGCCAAGTCAGCTCGCTCTGTCCGTGCCCAGCGCCACACCGAC |
| | ATGCCCAAGACCCAGAAGGAAGTACATTTGAAGAACGCAAGTAGAGGG |
| | AGTGCAGGAAACAAGAACTACAGGATGTAG (SEQ ID NO: 61) |
| TGF-β1 | ATGCCGCCCTCCGGGCTGCGGCTGCTGCTGCTGCTGCTACCGCTGCTG |
| | TGGCTACTGGTGCTGACGCCTGGCCGGCCGGCCGCGGGACTATCCACC |
| | TGCAAGACTATCGACATGGAGCTGGTGAAGCGGAAGCGCATCGAGGCC |
| | ATCCGCGGCCAGATCCTGTCCAAGCTGCGGCTCGCCAGCCCCCCGAGC |
| | CAGGGGGAGGTGCCGCCCGGCCCGCTGCCCGAGGCCGTGCTCGCCCTG |
| | TACAACAGCACCCGCGACCGGGTGGCCGGGGAGAGTGCAGAACCGGAG |
| | CCCGAGCCTGAGGCCGACTACTACGCCAAGGAGGTCACCCGCGTGCTA |
| | ATGGTGGAAACCCACAACGAAATCTATGACAAGTTCAAGCAGAGTACA |
| | CACAGCATATATATGTTCTTCAACACATCAGAGCTCCGAGAAGCGGTA |
| | CCTGAACCCGTGTTGCTCTCCCGGGCAGAGCTGCGTCTGCTGAGGCTC |
| | AAGTTAAAAGTGGAGCAGCACGTGGAGCTGTACCAGAAATACAGCAAC |
| | AATTCCTGGCGATACCTCAGCAACCGGCTGCTGGCACCCAGCGACTCG |
| | CCAGAGTGGTTATCTTTTGATGTCACCGGAGTTGTGCGGCAGTGGTTG |
| | AGCCGTGGAGGGGAAATTGAGGGCTTTCGCCTTAGCGCCCACTGCTCC |
| | TGTGACAGCAGGGATAACACACTGCAAGTGGACATCAACGGGTTGAGT |
| | ACCGGCCGCCGAGGTGACCTGGCCACCATTCATGGCATGAACCGGCCT |
| | TTCCTGCTTCTCATGGCCACCCCGCTGGAGAGGGCCCAGCATCTGCAA |
| | AGCTCCCGGCACCGCCGAGCCCTGGACACCAACTATTGCTTCAGCTCC |
| | ACGGAGAAGAACTGCTGCGTGCGGCAGCTGTACATTGACTTCCGCAAG |
| | GACCTCGGCTGGAAGTGGATCCACGAGCCCAAGGGCTACCATGCCAAC |
| | TTCTGCCTCGGGCCCTGCCCCTACATTTGGAGCCTGGACACGCAGTAC |
| | AGCAAGGTCCTGGCCCTGTACAACCAGCATAACCCGGGCGCCTCGGCG |
| | GCGCCGTGCTGCGTGCCGCAGGCGCTGGAGCCGCTGCCCATCGTGTAC |
| | TACGTGGGCCGCAAGCCCAAGGTGGAGCAGCTGTCCAACATGATCGTG |
| | CGCTCCTGCAAGTGCAGC (SEQ ID NO: 62) |
| Hemoglobin | ACTCTTCTGGTCCCCACAGACTCAGAGAGAACCCACCATGGTGCTGTC |
| (α-globin) | TCCTGCCGACAAGACCAACGTCAAGGCCGCCTGGGGTAAGGTCGGCGC |
| | GCACGCTGGCGAGTATGGTGCGGAGGCCCTGGAGAGGATGTTCCTGTC |
| | CTTCCCCACCACCAAGACCTACTTCCCGCACTTCGACCTGAGCCACGG |
| | CTCTGCCCAGGTTAAGGGCCACGGCAAGAAGGTGGCCGACGCGCTGAC |
| | CAACGCCGTGGCGCACGTGGACGACATGCCCAACGCGCTGTCCGCCCT |
| | GAGCGACCTGCACGCGCACAAGCTTCGGGTGGACCCGGTCAACTTCAA |
| | GCTCCTAAGCCACTGCCTGCTGGTGACCCTGGCCGCCCACCTCCCCGC |
| | CGAGTTCACCCCTGCGGTGCACGCCTCCCTGGACAAGTTCCTGGCTTC |
| | TGTGAGCACCGTGCTGACCTCCAAATACCGTTAAGCTGGAGCCTCGGT |
| | GGCCATGCTTCTTGCCCCTTGGGCCTCCCCCCAGCCCCTCCTCCCCTT |
| | CCTGCACCCGTACCCCCGTGGTCTTTGAATAAAGTCTGAGTGGGCGGC |
| | A (SEQ ID NO: 63) |
| Hemoglobin | ACATTTGCTTCTGACACAACTGTGTTCACTAGCAACCTCAAACAGACA |
| (β-globin) | CCATGGTGCATCTGACTCCTGAGGAGAAGTCTGCCGTTACTGCCCTGT |
| | GGGGCAAGGTGAACGTGGATGAAGTTGGTGGTGAGGCCCTGGGCAGGC |
| | TGCTGGTGGTCTACCCTTGGACCCAGAGGTTCTTTGAGTCCTTTGGGG |
| | ATCTGTCCACTCCTGATGCTGTTATGGGCAACCCTAAGGTGAAGGCTC |
| | ATGGCAAGAAAGTGCTCGGTGCCTTTAGTGATGGCCTGGCTCACCTGG |
| | ACAACCTCAAGGGCACCTTTGCCACACTGAGTGAGCTGCACTGTGACA |
| | AGCTGCACGTGGATCCTGAGAACTTCAGGCTCCTGGGCAACGTGCTGG |
| | TCTGTGTGCTGGCCCATCACTTTGGCAAAGAATTCACCCCACCAGTGC |
| | AGGCTGCCTATCAGAAAGTGGTGGCTGGTGTGGCTAATGCCCTGGCCC |
| | ACAAGTATCACTAAGCTCGCTTTCTTGCTGTCCAATTTCTATTAAAGG |
| | TTCCTTTGTTCCCTAAGTCCAACTACTAAACTGGGGGATATTATGAAG |
| | GGCCTTGAGCATCTGGATTCTGCCTAATAAAAAACATTTATTTTCATT |
| | GCAA (SEQ ID NO: 64) |
| bFGF | CGGCCCCAGAAAACCCGAGCGAGTAGGGGGCGGCGCGCAGGAGGGAGG |
| | AGAACTGGGGGCGCGGGAGGCTGGTGGGTGTGGGGGGTGGAGATGTAG |
| | AAGATGTGACGCCGCGGCCCGGCGGGTGCCAGATTAGCGGACGCGGTG |
| | CCCGCGGTTGCAACGGGATCCCGGGCGCTGCAGCTTGGGAGGCGGCTC |
| | TCCCCAGGCGGCGTCCGCGGAGACACCCATCCGTGAACCCCAGGTCCC |
| | GGGCCGCCGGCTCGCCGCGCACCAGGGGCCGGCGGACAGAAGAGCGGC |
| | CGAGCGGCTCGAGGCTGGGGGACCGCGGGCGCGGCCGCGCGCTGCCGG |
| | GCGGGAGGCTGGGGGGCCGGGGCCGGGGCCGTGCCCCGGAGCGGGTCG |
| | GAGGCCGGGGCCGGGGCCGGGGGACGGCGGCTCCCCGCGCGGCTCCAG |
| | CGGCTCGGGGATCCCGGCCGGGCCCCGCAGGGACCATGGCAGCCGGGA |
| | GCATCACCACGCTGCCCGCCTTGCCCGAGGATGGCGGCAGCGGCGCCT |
| | TCCCGCCCGGCCACTTCAAGGACCCCAAGCGGCTGTACTGCAAAAACG |
| | GGGGCTTCTTCCTGCGCATCCACCCCGACGGCCGAGTTGACGGGGTCC |
| | GGGAGAAGAGCGACCCTCACATCAAGCTACAACTTCAAGCAGAAGAGA |
| | GAGGAGTTGTGTCTATCAAAGGAGTGTGTGCTAACCGTTACCTGGCTA |
| | TGAAGGAAGATGGAAGATTACTGGCTTCTAAATGTGTTACGGATGAGT |
| | GTTTCTTTTTTGAACGATTGGAATCTAATAACTACAATACTTACCGGT |
| | CAAGGAAATACACCAGTTGGTATGTGGCACTGAAACGAACTGGGCAGT |
| | ATAAACTTGGATCCAAAACAGGACCTGGGCAGAAAGCTATACTTTTTC |
| | TTCCAATGTCTGCTAAGAGCTGATTTTAATGGCCACATCTAATCTCAT |
| | TTCACATGAAAGAAGAAGTATATTTTAGAAATTTGTTAATGAGAGTAA |
| | AAGAAAATAAATGTGTATAGCTCAGTTTGGATAATTGGTCAAACAATT |
| | TTTTATCCAGTAGTAAAATATGTAACCATTGTCCCAGTAAAGAAAAAT |
| | AACAAAAGTTGTAAAATGTATATTCTCCCTTTTATATTGCATCTGCTG |
| | TTACCCAGTGAAGCTTACCTAGAGCAATGATCTTTTTCACGCATTTGC |
| | TTTATTCGAAAAGAGGCTTTTAAAATGTGCATGTTTAGAAACAAAATT |
| | TCTTCATGGAAATCATATACATTAGAAAATCACAGTCAGATGTTTAAT |
| | CAATCCAAAATGTCCACTATTTCTTATGTCATTCGTTAGTCTACATGT |
| | TTCTAAACATATAAATGTGAATTTAATCAATTCCTTTCATAGTTTTAT |
| | AATTCTCTGGCAGTTCCTTATGATAGAGTTTATAAAACAGTCCTGTGT |
| | AAACTGCTGGAAGTTCTTCCACAGTCAGGTCAATTTTGTCAAACCCTT |
| | CTCTGTACCCATACAGCAGCAGCCTAGCAACTCTGCTGGTGATGGGAG |
| | TTGTATTTTCAGTCTTCGCCAGGTCATTGAGATCCATCCACTCACATC |
| | TTAAGCATTCTTCCTGGCAAAAATTTATGGTGAATGAATATGGCTTTA |
| | GGCGGCAGATGATATACATATCTGACTTCCCAAAAGCTCCAGGATTTG |
| | TGTGCTGTTGCCGAATACTCAGGACGGACCTGAATTCTGATTTTATAC |
| | CAGTCTCTTCAAAAACTTCTCGAACCGCTGTGTCTCCTACGTAAAAAA |
| | AGAGATGTAGAAATCAATAATAATTACACTTTTAGAAACTGTATCATC |
| | AAAGATTTTCAGTTAAAGTAGCATTATGTAAAGGCTCAAAACATTACC |
| | CTAACAAAGTAAAGTTTTCAATACAAATTCTTTGCCTTGTGGATATCA |
| | AGAAATCCCAAAATATTTTCTTACCACTGTAAATTCAAGAAGCTTTTG |
| | AAATGCTGAATATTTCTTTGGCTGCTACTTGGAGGCTTATCTACCTGT |
| | ACATTTTTGGGGTCAGCTCTTTTTAACTTCTTGCTGCTCTTTTTCCCA |
| | AAAGGTAAAAATATAGATTGAAAAGTTAAAACATTTTGCATGGCTGCA |
| | GTTCCTTTGTTTCTTGAGATAAGATTCCAAAGAACTTAGATTCATTTC |
| | TTCAACACCGAAATGCTGGAGGTGTTTGATCAGTTTTCAAGAAACTTG |
| | GAATATAAATAATTTTATAATTCAACAAAGGTTTTCACATTTTATAAG |
| | GTTGATTTTTCAATTAAATGCAAATTTGTGTGGCAGGATTTTTATTGC |
| | CATTAACATATTTTTGTGGCTGCTTTTTCTACACATCCAGATGGTCCC |
| | TCTAACTGGGCTTTCTCTAATTTTGTGATGTTCTGTCATTGTCTCCCA |
| | AAGTATTTAGGAGAAGCCCTTTAAAAAGCTGCCTTCCTCTACCACTTT |
| | GCTGGAAAGCTTCACAATTGTCACAGACAAAGATTTTTGTTCCAATAC |
| | TCGTTTTGCCTCTATTTTTCTTGTTTGTCAAATAGTAAATGATATTTG |
| | CCCTTGCAGTAATTCTACTGGTGAAAAACATGCAAAGAAGAGGAAGTC |
| | ACAGAAACATGTCTCAATTCCCATGTGCTGTGACTGTAGACTGTCTTA |
| | CCATAGACTGTCTTACCCATCCCCTGGATATGCTCTTGTTTTTTCCCT |
| | CTAATAGCTATGGAAAGATGCATAGAAAGAGTATAATGTTTTAAAACA |
| | TAAGGCATTTGTCTGCCATTTTTCAATTACATGCTGACTTCCCTTACA |
| | ATTGAGATTTGCCCATAGGTTAAACATGGTTAGAAACAACTGAAAGCA |
| | TAAAAGAAAAATCTAGGCCGGGTGCAGTGGCTCATGCCTATATTCCCT |
| | GCACTTTGGGAGGCCAAAGCAGGAGGATCGCTTGAGCCCAGGAGTTCA |
| | AGACCAACCTGGTGAAACCCCGTCTCTACAAAAAAACACAAAAAATAG |
| | CCAGGCATGGTGGCGTGTACATGTGGTCTCAGATACTTGGGAGGCTGA |
| | GGTGGGAGGGTTGATCACTTGAGGCTGAGAGGTCAAGGTTGCAGTGAG |
| | CCATAATCGTGCCACTGCAGTCCAGCCTAGGCAACAGAGTGAGACTTT |
| | GTCTCAAAAAAAGAGAAATTTTCCTTAATAAGAAAAGTAATTTTTACT |
| | CTGATGTGCAATACATTTGTTATTAAATTTATTATTTAAGATGGTAGC |
| | ACTAGTCTTAAATTGTATAAAATATCCCCTAACATGTTTAAATGTCCA |
| | TTTTTATTCATTATGCTTTGAAAAATAATTATGGGGAAATACATGTTT |
| | GTTATTAAATTTATTATTAAAGATAGTAGCACTAGTCTTAAATTTGAT |
| | ATAACATCTCCTAACTTGTTTAAATGTCCATTTTTATTCTTTATGTTT |
| | GAAAATAAATTATGGGGATCCTATTTAGCTCTTAGTACCACTAATCAA |
| | AAGTTCGGCATGTAGCTCATGATCTATGCTGTTTCTATGTCGTGGAAG |
| | CACTGGATGGGGGTAGTGAGCAAATCTGCCCTGCTCAGCAGTCACCAT |
| | AGCAGCTGACTGAAAATCAGCACTGCCTGAGTAGTTTTGATCAGTTTA |
| | ACTTGAATCACTAACTGACTGAAAATTGAATGGGCAAATAAGTGCTTT |
| | TGTCTCCAGAGTATGCGGGAGACCCTTCCACCTCAAGATGGATATTTC |
| | TTCCCCAAGGATTTCAAGATGAATTGAAATTTTTAATCAAGATAGTGT |
| | GCTTTATTCTGTTGTATTTTTTATTATTTTAATATACTGTAAGCCAAA |
| | CTGAAATAACATTTGCTGTTTTATAGGTTTGAAGAACATAGGAAAAAC |
| | TAAGAGGTTTTGTTTTTATTTTTGCTGATGAAGAGATATGTTTAAATA |
| | TGTTGTATTGTTTTGTTTAGTTACAGGACAATAATGAAATGGAGTTTA |
| | TATTTGTTATTTCTATTTTGTTATATTTAATAATAGAATTAGATTGAA |
| | ATAAAATATAATGGGAAATAATCTGCAGAATGTGGGTTTTCCTGGTGT |
| | TTCCCTCTGACTCTAGTGCACTGATGATCTCTGATAAGGCTCAGCTGC |
| | TTTATAGTTCTCTGGCTAATGCAGCAGATACTCTTCCTGCCAGTGGTA |
| | ATACGATTTTTTAAGAAGGCAGTTTGTCAATTTTAATCTTGTGGATAC |
| | CTTTATACTCTTAGGGTATTATTTTATACAAAAGCCTTGAGGATTGCA |
| | TTCTATTTTCTATATGACCCTCTTGATATTTAAAAAACACTATGGATA |
| | ACAATTCTTCATTTACCTAGTATTATGAAAGAATGAAGGAGTTCAAAC |
| | AAATGTGTTTCCCAGTTAACTAGGGTTTACTGTTTGAGCCAATATAAA |
| | TGTTTAACTGTTTGTGATGGCAGTATTCCTAAAGTACATTGCATGTTT |
| | TCCTAAATACAGAGTTTAAATAATTTCAGTAATTCTTAGATGATTCAG |
| | CTTCATCATTAAGAATATCTTTTGTTTTATGTTGAGTTAGAAATGCCT |
| | TCATATAGACATAGTCTTTCAGACCTCTACTGTCAGTTTTCATTTCTA |
| | GCTGCTTTCAGGGTTTTATGAATTTTCAGGCAAAGCTTTAATTTACAC |
| | TAAGCTTAGGAAGTATGGCTAATGCCAACGGCAGTTTTTTTCTTCTTA |
| | ATTCCACATGACTGAGGCATATATGATCTCTGGGTAGGTGAGTTGTTG |
| | TGACAACCACAAGCACTTTTTTTTTTTTTAAAGAAAAAAAGGTAGTGA |
| | ATTTTTAATCATCTGGACTTTAAGAAGGATTCTGGAGTATACTTAGGC |
| | CTGAAATTATATATATTTGGCTTGGAAATGTGTTTTTCTTCAATTACA |
| | TCTACAAGTAAGTACAGCTGAAATTCAGAGGACCCATAAGAGTTCACA |
| | TGAAAAAAATCAATTTATTTGAAAAGGCAAGATGCAGGAGAGAGGAAG |
| | CCTTGCAAACCTGCAGACTGCTTTTTGCCCAATATAGATTGGGTAAGG |
| | CTGCAAAACATAAGCTTAATTAGCTCACATGCTCTGCTCTCACGTGGC |
| | ACCAGTGGATAGTGTGAGAGAATTAGGCTGTAGAACAAATGGCCTTCT |
| | CTTTCAGCATTCACACCACTACAAAATCATCTTTTATATCAACAGAAG |
| | AATAAGCATAAACTAAGCAAAAGGTCAATAAGTACCTGAAACCAAGAT |
| | TGGCTAGAGATATATCTTAATGCAATCCATTTTCTGATGGATTGTTAC |
| | GAGTTGGCTATATAATGTATGTATGGTATTTTGATTTGTGTAAAAGTT |
| | TTAAAAATCAAGCTTTAAGTACATGGACATTTTTAAATAAAATATTTA |
| | AAGACAATTTAGAAAATTGCCTTAATATCATTGTTGGCTAAATAGAAT |
| | AGGGGACATGCATATTAAGGAAAAGGTCATGGAGAAATAATATTGGTA |
| | TCAAACAAATACATTGATTTGTCATGATACACATTGAATTTGATCCAA |
| | TAGTTTAAGGAATAGGTAGGAAAATTTGGTTTCTATTTTTCGATTTCC |
| | TGTAAATCAGTGACATAAATAATTCTTAGCTTATTTTATATTTCCTTG |
| | TCTTAAATACTGAGCTCAGTAAGTTGTGTTAGGGGATTATTTCTCAGT |
| | TGAGACTTTCTTATATGACATTTTAGTATGTTTTGACTAGCTGACTAT |
| | TAAAAATAAATAGTAGATACAATTTTCATAAAGTGAAGAATTATATAA |
| | TGAGTGCTTTATAACTGAGTTTATTATATTTATTTCAAAGTTCATTTA |
| | AAGGCTACTATTCATCCTCTGTGATGGAATGGTCAGGAATTTGTTTTC |
| | TCATAGTTTAATTCCAACAACAATATTAGTCGTATCCAAAATAACCTT |
| | TAATGCTAAACTTTACTGATGTATATCCAAAGCTTCTCATTTTCAGAC |
| | AGATTAATCCAGAAGCAGTCATAAACAGAAGAATAGGTGGTATGTTCC |
| | TAATGATATTATTTCTACTAATGGAATAAACTGTAATATTAGAAATTA |
| | TGCTGCTAATTATATCAGCTCTGAGGTAATTTCTGAAATGTTCAGACT |
| | CAGTCGGAACAAATTGGAAAATTTAAATTTTTATTCTTAGCTATAAAG |
| | CAAGAAAGTAAACACATTAATTTCCTCAACATTTTTAAGCCAATTAAA |
| | AATATAAAAGATACACACCAATATCTTCTTCAGGCTCTGACAGGCCTC |
| | CTGGAAACTTCCACATATTTTTCAACTGCAGTATAAAGTCAGAAAATA |
| | AAGTTAACATAACTTTCACTAACACACACATATGTAGATTTCACAAAA |
| | TCCACCTATAATTGGTCAAAGTGGTTGAGAATATATTTTTTAGTAATT |
| | GCATGCAAAATTTTTCTAGCTTCCATCCTTTCTCCCTCGTTTCTTCTT |
| | TTTTTGGGGGAGCTGGTAACTGATGAAATCTTTTCCCACCTTTTCTCT |
| | TCAGGAAATATAAGTGGTTTTGTTTGGTTAACGTGATACATTCTGTAT |
| | GAATGAAACATTGGAGGGAAACATCTACTGAATTTCTGTAATTTAAAA |
| | TATTTTGCTGCTAGTTAACTATGAACAGATAGAAGAATCTTACAGATG |
| | CTGCTATAAATAAGTAGAAAATATAAATTTCATCACTAAAATATGCTA |
| | TTTTAAAATCTATTTCCTATATTGTATTTCTAATCAGATGTATTACTC |
| | TTATTATTTCTATTGTATGTGTTAATGATTTTATGTAAAAATGTAATT |
| | GCTTTTCATGAGTAGTATGAATAAAATTGATTAGTTTGTGTTTTCTTG |
| | TCTCCCAAAAAAA (SEQ ID NO: 65) |

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A nucleic acid composition comprising (a) a first expression cassette comprising a pol II promoter operably linked to a coding sequence for a therapeutic protein, wherein the pol II promoter is a constitutive promoter; (b) a second expression cassette comprising a pol II promoter operably linked to a coding sequence of a dCas9-activator protein; and (c) a third expression cassette comprising a pol III promoter operably linked to a coding sequence for a guide RNA (gRNA), wherein the gRNA is able to hybridize to the pol II promoter that is present in both the first and second expression cassettes.

2. The nucleic acid composition of claim 1, wherein:
the pol II promoter in a) and in b) are the same promoter; or
the pol II promoter in a) and in b) are different, wherein the gRNA is able to hybridize to the pol II promoters in a) and in b).

3. (canceled)

4. The nucleic acid composition of claim 1, wherein each expression cassette is located on a separate nucleic acid molecule, or on a single nucleic acid molecule.

5. The nucleic acid composition of claim 4, wherein the nucleic acid molecules on which the first, the second, and the third expression cassettes are located each comprise an antibiotic resistance gene, wherein the antibiotic resistance genes on the first, the second, and the third expression cassettes are different.

6. (canceled)

7. The nucleic acid composition of claim 5, wherein the antibiotic resistance gene on one or more of the nucleic acid molecules is flanked by a loxP site.

8-11. (canceled)

12. The nucleic acid composition of claim 1, wherein the pol II promoter is a cell-type specific promoter.

13. The nucleic acid composition of claim 1, wherein the pol II promoter is an EF-1a promoter, a CMV promoter, a Ubc promoter, a PGK promoter, a VMD2 promoter, or a CAG promoter.

14. The nucleic acid composition of claim 1, wherein the pol III promoter is a U6 promoter.

15. The nucleic acid composition of claim 1, wherein the first and/or second and/or third expression cassette comprises an enhancer element, and wherein the first and/or second and/or third expression cassette comprises a filler polynucleotide.

16. The nucleic acid composition of claim 1, wherein the first and/or second expression cassette comprises an intron, a poly A signal, or a combination thereof.

17. (canceled)

18. The nucleic acid composition of claim 1, wherein the therapeutic protein is human IL-2, VEGF alpha, TGF beta, IL-4, IL-6, IL-7, IL-10, IL-12a, IL-12b, IL-15, Factor VII, Factor VIII, VWF, GCG, Factor IX, proenkephalin, EGF, IGF-1, TGF-beta1, hemoglobin (alpha-globin), hemoglobin (beta-globin), or bFGF.

19. The nucleic acid composition of claim 1, wherein the first and/or second and/or third expression cassette is comprised in a viral vector.

20. The nucleic acid composition of claim 19, wherein the viral vector is selected from an adeno-associated viral (AAV) vector, a lentiviral vector, or a retroviral vector.

21. A cell comprising the nucleic acid composition of claim 1.

22-25. (canceled)

26. A pharmaceutical composition comprising the cell of claim 21 and a pharmaceutically acceptable carrier.

27. (canceled)

28. A method of producing a therapeutic protein, the method comprising culturing the cells of claim 21 under conditions to allow for expression of the therapeutic protein and isolating the therapeutic protein from the cells.

29. A recombinant adeno-associated virus (rAAV) vector comprising an AAV capsid protein and the nucleic acid composition of claim 1.

30. The rAAV of claim 29, wherein the AAV vector comprises an AAV particle comprising AAV capsid proteins, and wherein the first and/or second and/or third expression cassette is inserted between a pair of AAV inverted terminal repeats (ITRs).

31-32. (canceled)

33. A method of treating a disease in a subject in need thereof, the method comprising administering to the subject a therapeutically effective amount of a nucleic acid composition of claim 1.

34-42. (canceled)

43. A method of treating a disease in a subject in need thereof, the method comprising administering to the subject a therapeutically effective amount of the cells of claim 21.

44-93. (canceled)
